(19) United States
(12) Patent Application Publication
(10) Pub. No.: US 2023/0069856 A1
(43) Pub. Date: Mar. 9, 2023

(54) Blood Plasma Fractions for Improvement of Myelination

(71) Applicant: Alkahest, Inc., San Carlos, CA (US)

(72) Inventors: David Bell, Dublin (IE); Steven P. Braithwaite, Redwood City, CA (US); Vu Dang, San Mateo, CA (US); Ian Gallager, Half Moon Bay, CA (US); Joe McCracken, Hillsborough, CA (US); S. Sakura Minami, San Francisco, CA (US); Karoly Nikolich, Emerald Hills, CA (US); Jung Hyung Sin, Cupertino, CA (US)

(21) Appl. No.: 17/985,721

(22) Filed: Nov. 11, 2022

Related U.S. Application Data
(63) Continuation-in-part of application No. 17/582,974, filed on Jan. 24, 2022.
Application No. 17/582,974 is a continuation of application No. 16/456,717, filed on Jun. 28, 2019, now abandoned.
Application No. 16/456,717 is a continuation of application No. 15/499,694, filed on Apr. 27, 2017, now Pat. No. 10,525,107.
Application No. 15/499,694 is a continuation-in-part of application No. 17/115,144, filed on Dec. 8, 2020, now Pat. No. 11,547,724.
Application No. 17/115,144 is a division of application No. 16/659,000, filed on Oct. 21, 2019, now Pat. No. 11,103,530.
(60) Provisional application No. 62/412,258, filed on Oct. 24, 2016.
(60) Provisional application No. 62/376,529, filed on Aug. 18, 2016.
(60) Provisional application No. 62/842,403, filed on May 2, 2019.
(60) Provisional application No. 62/751,448, filed on Oct. 26, 2018.

(51) Int. Cl. A61K 38/38; A61K 35/16

(52) U.S. Cl. CPC A61K 38/385; A61K 35/16

(57) ABSTRACT

Methods and compositions for restoring myelin levels in conditions associated with myelin degeneration, for example, an aging and aging-related neurodegenerative and/or neuroinflammatory disease or a myelopathy associated with postoperative recovery. The compositions used in the methods include blood plasma and blood plasma fractions derived from blood plasma with efficacy in restoring myelin levels and/or improving nerve conductance.

### CROSS REFERENCE TO RELATED APPLICATIONS

This application is a continuation-in-part application of U.S. patent application Ser. No. 17/582,974, filed on Jan. 24, 2022; which application is a continuation application of U.S. patent application Ser. No. 16/456,717, filed on Jun. 28, 2019 and now abandoned; which application is a continuation application of U.S. patent application Ser. No. 15/499,694, filed on Apr. 27, 2017 and now issued as U.S. Pat. No. 10,525,107; which application, pursuant to 35 U.S.C. § 119 (e), claims priority to U.S. Provisional Patent Application Ser. No. 62/412,258, filed Oct. 24, 2016 and U.S. Provisional Patent Application Ser. No. 62/376,529, filed Aug. 18, 2016; and this application is a continuation-in-part application of U.S. patent application Ser. No. 17/115,144, filed on Dec. 8, 2020; which application is a divisional application of U.S. patent application Ser. No. 16/659,000, filed on Oct. 21, 2019 and now issued as U.S. Pat. No. 11,103,530; which application, pursuant to 35 U.S.C. § 119 (e), claims priority to U.S. Provisional Patent Application Ser. No. 62/842,403, filed May 2, 2019, and U.S. Provisional Patent Application Ser. No. 62/751,448, filed Oct. 26, 2018; the disclosures of which applications are herein incorporated by reference.

### FIELD OF THE INVENTION

This invention pertains to the prevention and treatment of certain aging-associated diseases. The invention also relates to the use of blood products, such as blood plasma and blood plasma fractions to improve and accelerate recovery from surgery, including conditions and indications related to surgery. The invention further relates to the use of blood products, such as blood plasma and blood plasma fractions to alleviate chronic pain or neuropathy and to treat indications related to wound healing. Furthermore, the invention relates to the use of blood products, such as blood plasma and blood plasma fractions to treat and/or prevent conditions associated with aging, such as neurocognitive and neurodegenerative disorders. Even further, the invention relates to the use of blood products, such as blood plasma and blood plasma fractions to restore myelin levels in conditions associated with myelin degeneration, for example, aging and aging-related neuroinflammatory diseases. Restoring myelin levels in such diseases improves nerve conductance. Accordingly, this invention pertains to methods of improving nerve myelination and/or improving nerve conductance by administering a blood plasma or blood plasma fraction to a subject having a condition associated with myelin degeneration.

### BACKGROUND

The following is offered as background information only and is not admitted as prior art to the present invention.

Aging is an important risk factor for multiple human diseases including cognitive impairment, cancer, arthritis, vision loss, osteoporosis, diabetes, cardiovascular disease, and stroke. In addition to normal synapse loss during natural aging, synapse loss is an early pathological event common to many neurodegenerative conditions, and is the best correlate to the neuronal and cognitive impairment associated with these conditions. As such, aging remains the single most dominant risk factor for dementia-related neurodegenerative diseases such as Alzheimer's disease (AD) (Bishop, N. A. et al., Neural mechanisms of ageing and cognitive decline. Nature 464(7288), 529-535 (2010); Heeden, T. et al., Insights into the ageing mind: a view from cognitive neuroscience. Nat. Rev. Neurosci. 5(2), 87-96 (2004); Mattson, M. P., et al., Ageing and neuronal vulnerability. Nat. Rev. Neurosci. 7(4), 278-294 (2006)). Aging affects all tissues and functions of the body including the central nervous system, and a decline in functions such as cognition, can severely impact quality of life. Treatment for cognitive decline and neurodegenerative disorders has had limited success in preventing and reversing impairment. It is therefore important to identify new treatments for maintaining cognitive integrity by protecting against, countering, or reversing the effects of aging.

Surgery is often associated with complications from pain, cardiopulmonary issues, infections, thromboembolic issues, and postsurgical wound healing. Additionally, it takes time for wounds to heal whether incurred from surgery itself (e.g. incisions) or incurred by accident, force, or disease and subsequently treated by a surgical procedure. Such complications are often further exacerbated by age. Additional complications may arise from the surgical stress response with subsequent demand on organ function, which are often mediated by trauma-induced endocrine metabolic changes and activation of cascades (cytokines, complement, arachidonic acid metabolites, nitric oxide, and free oxygen radicals). (Kehlet H., et al., Br. J. Anaesthesia, 78:606-17 (1997)). During surgical stress response, the sympathetic nervous system is activated. (Starkweather A, Topics in Pain Management, 32(8):1-11 (2017)). There is an increase in pituitary hormone secretion, resulting in mobilization of energy through catabolism. This in turn results in salt and water retention. Adrenocorticotropic hormone (ACTH) secretion is increased, which results in an increase of norepinephrine and sympathetic activity. This causes cardiovascular responses such as tachycardia and hypertension and glucagon is released resulting in hyperglycemia. An increase in growth hormone and cortisol also results in inhibition of monocyte to macrophage differentiation. This in turn, interferes with T-cell signaling/histamine production and decreases immune cell migration. (Id.)

Current treatment for postsurgical recovery includes reduction of postoperative pain as well as multimodal interventions. (Id.) Pain management is important in many types of surgical recoveries, and acute pain is expected. (Pinto P R, J Pain Res, 10:1087-98 (2017)). Postoperative pain is associated to a greater degree with patients who undergo general surgery. (Couceiro T C, Rev Bras Anestesiol, 59(3):314-20 (2009)). Pain also plays a negative role on clinical outcome because it impairs healing and recovery. Id. Replacement of the hip and knee joints is particularly associated with pain, both chronic (from, e.g. osteoarthritis) and acute. Id. Analgesics are therefore commonly used in postoperative recovery, both during in-patient procedures and home recovery.

One type of multimodal intervention is Enhanced Recovery After Surgery (ERAS). (Starkweather A, supra). ERAS focuses on a wide spectrum of surgeries, for example, colorectal surgery, orthopedics, gynecology, urology, head and neck cancer, bladder cancer, liver disease, rectal/pelvic disease, colonic pathologies, pancreative duodenectomy, gastrectomy, and bariatric and gynecologic-oncology surgery. Id. As a multimodal strategy, it emphasizes: pre-operative techniques (counseling, fluid/carbohydrate loading; shorter period of fasting); perioperative techniques (short-acting anesthetics; normothermia; antibiotic prophylaxis; thromboembolic prophylaxis; prevention of salt/water overload; vomiting prevention); and postoperative techniques (early oral diet; exercise; non-opioid analgesia; and post-discharge support). Id.

Current therapies however have failed to eliminate postoperative morbidity and mortality. Multimodal techniques by their very nature are time and resource consuming. And there has not been any single technique or pharmaceutical treatment that can match such multimodal therapy. Because of these shortfalls, there is a need for new treatments for improving postoperative recovery.

White matter degeneration is a critical component of aging, as the ability to repair and replace healthy cells that promote the normal myelin renewal process decreases over time. Maintaining myelin sheath integrity is important to ensure proper axonal function and efficient signal transduction in neurons, and loss of white matter contributes to cognitive impairment, specifically memory consolidation in many neurodegenerative conditions. Because of the consequences of myelin degeneration, there is a need for new treatments for restoring myelin levels, improving nerve myelination, and/or improving nerve conductance in conditions associated with myelin degeneration.

### SUMMARY

The present invention is based on the production and use of blood products for treating and/or preventing age-related disorders, such as cognitive impairment conditions, age-related dementia, and neurodegenerative disease. The present invention recognizes, among other things, the need for new therapies for the treatment and/or prevention of cognitive impairment, age-related dementia, and neurodegenerative disease. Derived from blood and blood plasma, the present compositions of the invention relate to a solution for the failures and shortcomings of current therapies through utilization of blood plasma fractions exhibiting efficacy in the treatment and/or prevention of cognitive impairment, age-related dementia, and neurodegenerative disease. Additionally, the current invention relates to proteins identified in blood plasma fractions which either may exhibit efficacy as treatments or preventative agents for cognitive impairment and age-related dementia themselves, or are targets for inhibition by additional agents.

The present invention is also based on the production and use of blood products for treating symptoms and conditions impacting surgical recovery including, for example, pain and wound healing. The present invention recognizes, among other things, the need for new therapies for the treatment of unwanted conditions associated with postoperative recovery, and for improving such recovery. Derived from blood and blood plasma, the present compositions of the invention relate to a solution for the failures and shortcomings of current therapies through utilization of blood plasma fractions exhibiting efficacy in the treatment of unwanted conditions associated with postsurgical recovery and for improving such recovery.

Further, the present invention relates to the use of blood products, such as blood plasma and blood plasma fractions, to restore myelin levels in conditions associated with myelin degeneration, for example, aging-associated neurocognitive and neurodegenerative disorders or a myelopathy associated with post-operative recovery. Restoring myelin levels in such diseases improves nerve conductance. Accordingly, certain embodiments of the invention pertain to methods of improving nerve myelination and/or improving nerve conductance by administering a blood plasma or blood plasma fraction to a subject having a condition associated with myelin degeneration, such as but not limited to, aging-associated neurocognitive and neurodegenerative disorders or a myelopathy associated with post-operative recovery.

The present invention also is based on the production and use of blood products for treating symptoms and conditions associated with acute and chronic pain. The present invention recognizes, among other things, the need for new therapies for alleviating pain. Although therapeutics exist for treating acute and chronic pain, many such therapies such as opioid analgesics present a high incidence of addiction, abuse, and associated morbidity and mortality.

The current invention also recognizes that differences in protein content between different blood plasma fractions (e.g. fractions, effluents, “Plasma Fractions,” Plasma Protein Fraction, Human Albumin Solution) can be responsible for preventing and/or improving certain cognitive impairments and alleviating neurodegenerative disease. By way of example, and not limitation, embodiments of the current invention demonstrate that mere higher albumin concentration of Human Albumin Solution (HAS) preparations is not the driving force behind improved cognition associated with Plasma Protein Fraction (PPF) preparations with lower albumin concentrations.

Blood and blood plasma from young donors have exhibited improvement and reversal of the pre-existing effects of brain aging, including at the molecular, structural, functional, and cognitive levels. (Saul A. Villeda, et al. Young blood reverses age-related impairments in cognitive function and synaptic plasticity in mice. Nature Medicine 20 659-663 (2014)). The present invention relates to fractions and effluents of the blood plasma, some of which have been traditionally used to treat patient shock, and the discovery that they are effective as methods of treatment of aging-associated cognitive impairment.

In accordance with aspects of the invention, then, methods of treatment of aging-associated cognitive impairment, age-related dementia, and/or neurodegenerative disease using blood product fractions of blood plasma are provided. Aspects of the methods include administering a blood plasma fraction to an individual suffering from or at risk of developing aging-associated cognitive impairment or neurodegenerative disease. Additional aspects of the methods include administering a blood plasma fraction derived from a pool of donors of a specific age range to an individual suffering from or at risk of developing aging-associated cognitive impairment. Also provided are reagents, devices, and kits thereof that find use in practicing the subject methods.

In an embodiment, the blood plasma fraction may be, for example, one of several blood plasma fractions obtained from a blood fractionation process, such as the Cohn fractionation process described below. In another embodiment, the blood plasma fraction may be of the type, herein referred to as “Plasma Fraction,” which is a solution comprised of normal human albumin, alpha and beta globulins, gamma globulin, and other proteins, either individually or as complexes. In another embodiment, the blood plasma fraction may be a type of blood plasma fraction known to those having skill in the art as a “Plasma Protein Fraction” (PPF). In another embodiment, the blood plasma fraction may be a “Human Albumin Solution” (HAS) fraction. In yet another embodiment, the blood plasma fraction may one in which substantially all of the clotting factors are removed in order to retain the efficacy of the fraction with reduced risk of thromboses. Embodiments of the invention may also include administering, for example, a fraction derived from a young donor or pools of young donors. Another embodiment of the invention may include the monitoring of cognitive improvement in a subject treated with a blood plasma fraction.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The accompanying drawings illustrate embodiments of the invention and, together with the description, serve to explain the invention. These drawings are offered by way of illustration and not by way of limitation: it is emphasized that the various features of the drawings may not be to-scale.

FIG. 1 shows the time spent rearing by control, PPF1, or HAS1-treated 3-month or 13-month-old NSG mice that were placed in an Open Field chamber for 15 minutes.

FIG. 2 shows the movement velocity of control, PPF1, or HAS1-treated 3-month or 13-month-old NSG mice that were placed in an Open Field chamber for 15 minutes.

FIG. 3 shows the movement distance traveled of control, PPF1, or HAS1-treated 3-month or 13-month-old NSG mice that were placed in an Open Field chamber for 15 minutes.

FIG. 4 shows the time spent in the novel arm by 3-month or 13-month-old NSG mice in the cued Y-maze test that were treated with control, PPF1, or HAS1.

FIG. 5 shows the ratio of time spent by 3-month or 13-month-old NSG mice in the novel versus familiar arms (ratio of novel:familiar) of the cued Y-maze test, the mice having been treated with control, PPF1, or HAS1.

FIG. 6 shows the movement velocity of control, PPF1, or HAS1-treated 3-month or 13-month-old NSG mice in the cued Y-maze test.

FIG. 7 shows the movement distance traveled of 3-month or 13-month-old NSG mice in the cued Y-maze test, the mice having been treated with control, PPF1, or HAS1.

FIG. 8A shows the percent of time freezing in the contextual fear conditioning test for memory by 3-month and 13-month-old NSG mice treated with control, PPF1, or HAS1.

FIG. 8B shows the percent of time freezing in the auditory cued fear conditioning test for memory by 3-month and 13-month-old NSG mice treated with control, PPF1, or HAS1.

FIG. 9 quantifies percent of time freezing during the last 90 seconds of the cued fear conditioning test for memory in 3-month and 13-month-old NSG mice treated with control, PPF1, or HAS1.

FIG. 10A charts the Barnes maze latency which tests for spatial memory. The latency to reach the target hole in 3-month and 13-month-old NSG mice treated with control, PPF1, or HAS1 is reported.

FIG. 10B quantifies the average of the last 3 trials depicted in FIG. 10A.

FIG. 11A quantifies the number of cells positively staining for Doublecortin (Dcx), a marker for newborn neurons in the dentate gyrus of 3-month and 13-month-old NSG mice treated with control, PPF1, or HAS1 twice per week for up to 6 months.

FIG. 11B quantifies the number of cells positively staining for Ki67, a marker for proliferating cells in the dentate gyrus of 3-month and 13-month-old NSG mice treated with control, PPF1, or HAS1 twice per week for up to 6 months.

FIG. 12 quantifies the number of cells positively staining for Dcx in 13-month-old NSG mice treated with control, PPF1, 1× concentrated HAS1, or 5× concentrated HAS1 three times per week for five weeks.

FIG. 13 quantifies the number of cells positively staining for Ki67 in 13-month-old NSG mice treated with control, PPF1, 1× concentrated HAS1, or 5× concentrated HAS1 three times per week for five weeks.

FIG. 14A shows the number of rearing bouts in an Open Field chamber in NODscid mice treated twice weekly via intravenous tail vein injection with either saline (control) or PPF1 starting at 6 months of age. Rearing was measured for a span of 15 minutes once mice were placed in the Open Field chamber.

FIG. 14B reports the movement velocity in an Open Field chamber of mice treated twice weekly via intravenous tail vein injection with either saline (control) or PPF1 starting at 6 months of age. Velocity was measured for a span of 15 minutes once mice were placed in the Open Field chamber.

FIG. 14C reports the distance traveled in an Open Field chamber of mice treated twice weekly via intravenous tail vein injection with either saline (control) or PPF1 starting at 6 months of age. Velocity was measured for a span of 15 minutes once mice were placed in the Open Field chamber.

FIG. 15 depicts the Barnes maze latency and hippocampal-dependent spatial learning and memory. The latency to reach the target hole in aged NSG mice (aged 12 months) treated with 150 μL saline control, young plasma, Effluent I, or Effluent II/III is reported.

FIG. 16 reports the effect of young human plasma, PPF1, and saline control on hippocampal-dependent spatial learning and memory in male aged NSG mice (aged 12 months). The mice were treated with 150 μL of clarified young human plasma (young plasma), PPF1, or saline three times per week (i.v.) for 4 weeks, and then twice per week during weeks 5 and 6, which were the weeks in which testing was performed is reported. The latency to reach the Barnes Maze hold for each treatment group is reported.

FIG. 17 reports the effect of young human plasma, PPF1, and saline control on the average latency to find the Barnes Maze target hole for the last three trials for each day of testing. Aged NSG mice (aged 12 months) were treated with 150 μL of clarified young human plasma (young plasma), PPF1, or saline three times per week (i.v.) for 4 weeks, and were subsequently treated twice per week during weeks 5 and 6, which were the weeks in which testing was performed.

FIG. 18 reports the effect of young human plasma, PPF1, and saline control on cell survival as determined by BrdU detection. Aged NSG mice (aged 12 months) were treated with 150 μL of clarified young human plasma (young plasma), PPF1, or saline three times per week (i.v.) for 4 weeks, and were subsequently treated twice per week during weeks 5 and 6, which were the weeks in which behavioral testing was performed. Hippocampal sections were analyzed after sacrifice.

FIG. 19 shows the effects of control, PPF1, and HAS1 on neurosphere proliferation in cortex culture. The figure shows example images of neurospheres from cortical cultures after 21 days in vitro, imaged for Tuj1, DAPI, or Tuj1 and DAPI.

FIG. 20 shows the effects of control, PPF1, and HAS1 on net neurite length in cortex culture.

FIG. 21 shows effects of vehicle, PPF1, and HAS1 on sphere and process growth in cortex culture. Yellow shading highlights spheres, and pink shading highlights neurites determined by an IncuCyte software algorithm (Essen BioScience, Inc., Ann Arbor, Mich.).

FIG. 22A shows the quantification of neurosphere number as percent of vehicle from cortices from E14-15 mouse embryos suspended in neural basal media supplemented with B27 and 2 mM Glutamax (vehicle), PPF1 (10% of a 5% stock solution), or HAS1 (10% of a 5% stock solution).

FIG. 21B shows the quantification of neurite length as percent of vehicle from cortices from E14-15 mouse embryos suspended in neural basal media supplemented with B27 and 2 mM Glutamax (vehicle), PPF1 (10% of a 5% stock solution), or HAS1 (10% of a 5% stock solution).

FIG. 21C shows the quantification of neurite branch points as percent of vehicle from cortices from E14-15 mouse embryos suspended in neural basal media supplemented with B27 and 2 mM Glutamax (vehicle), PPF1 (10% of a 5% stock solution), or HAS1 (10% of a 5% stock solution).

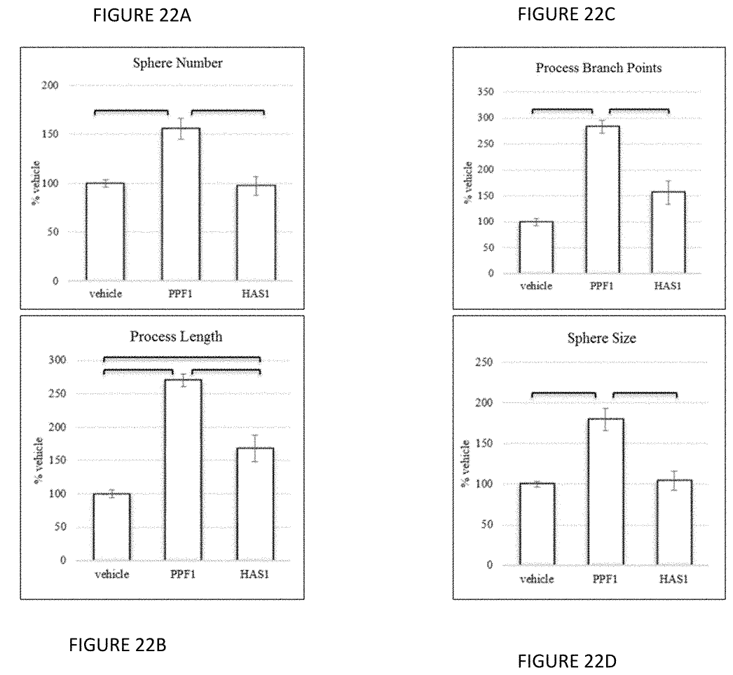

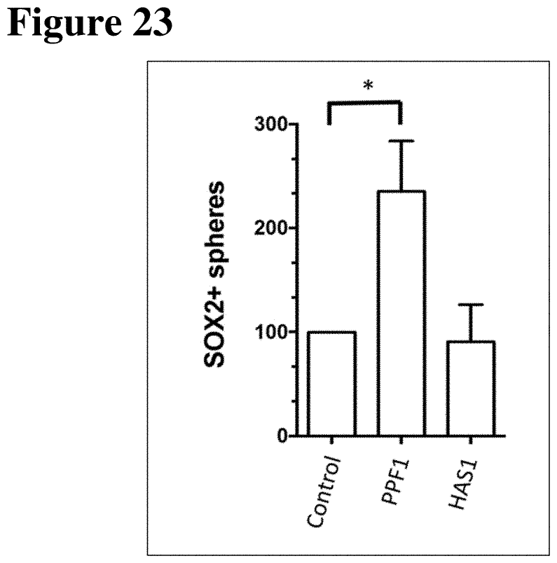

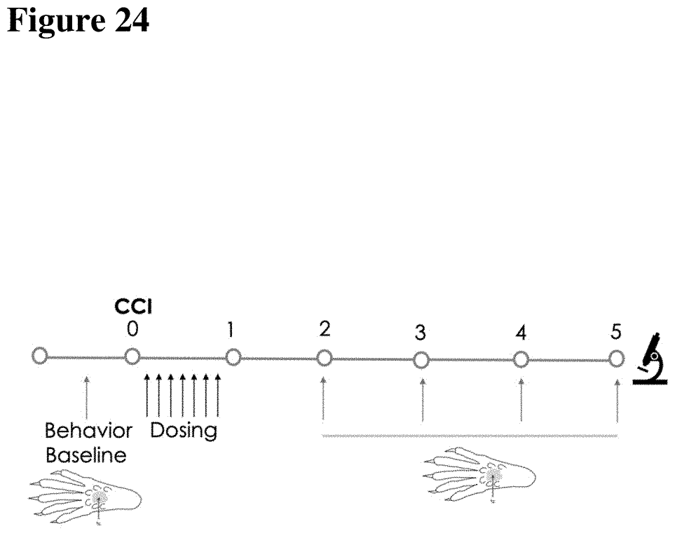

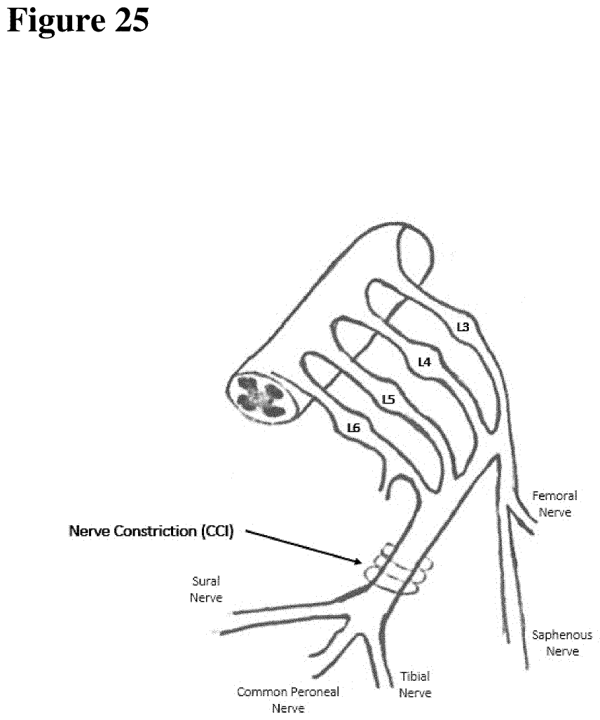

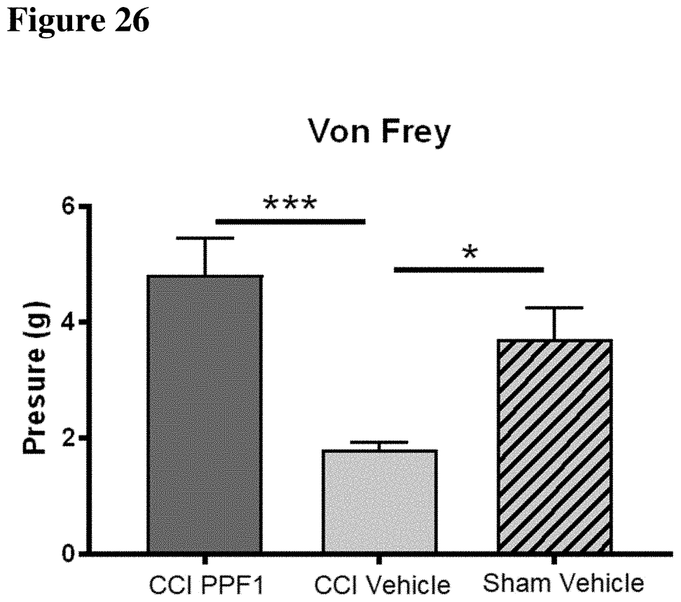

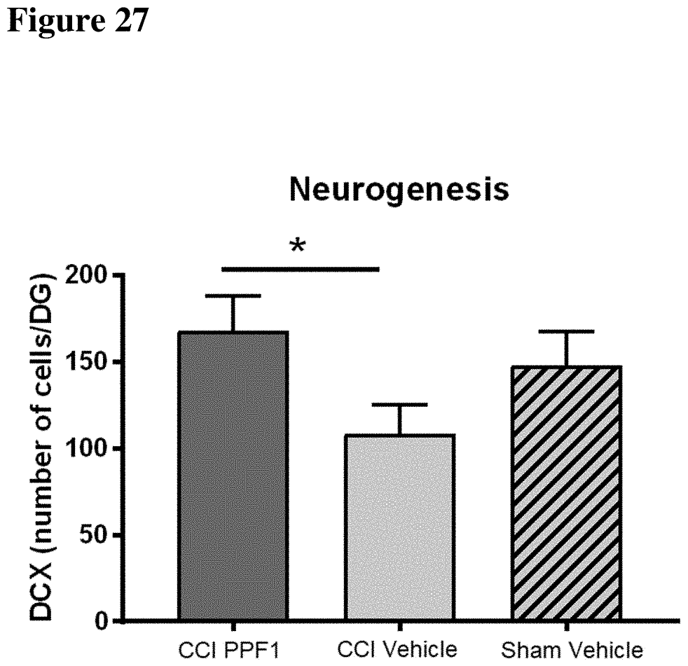

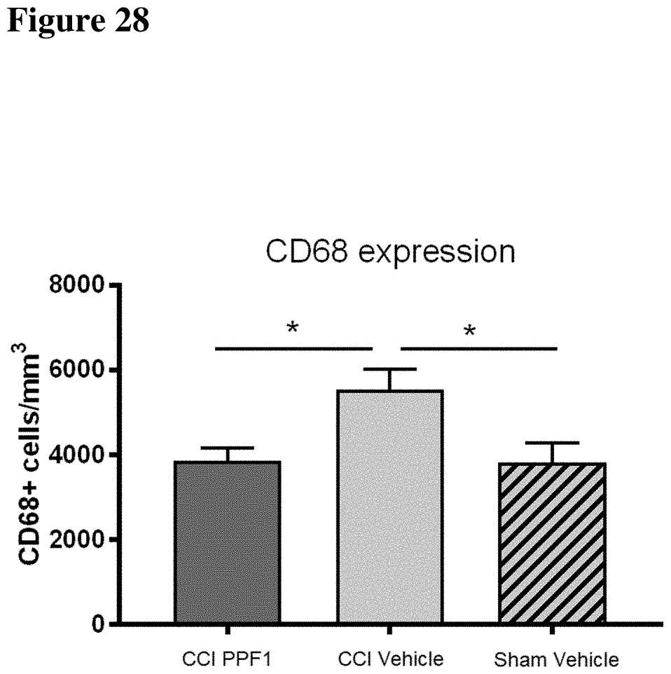

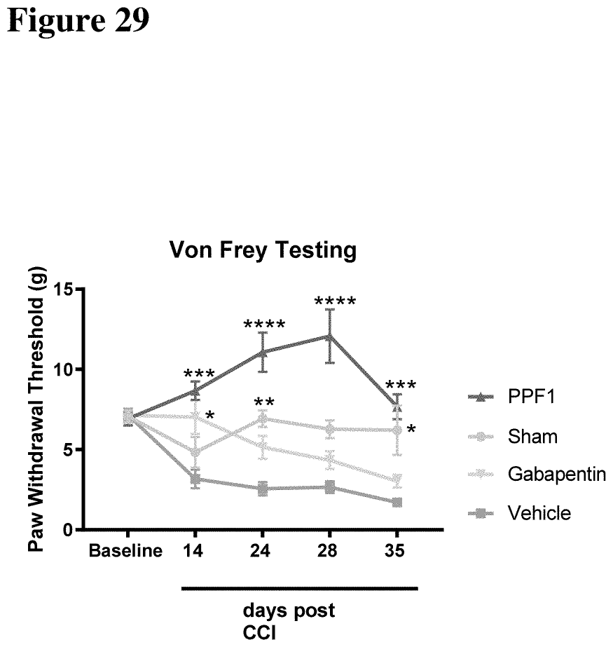

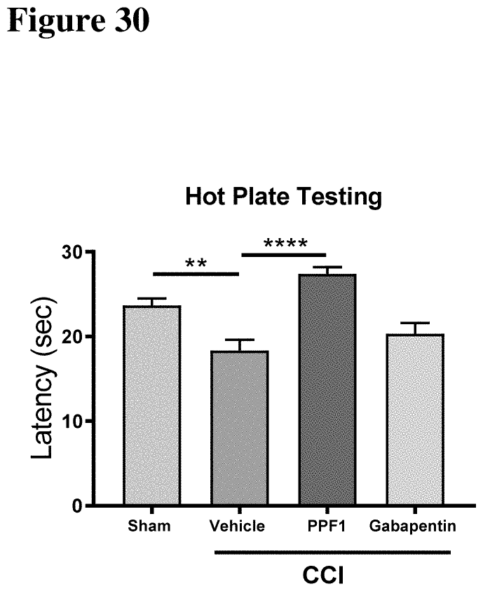

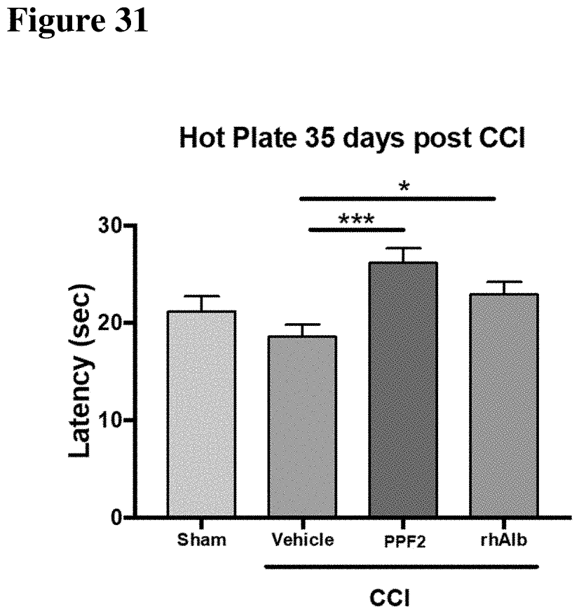

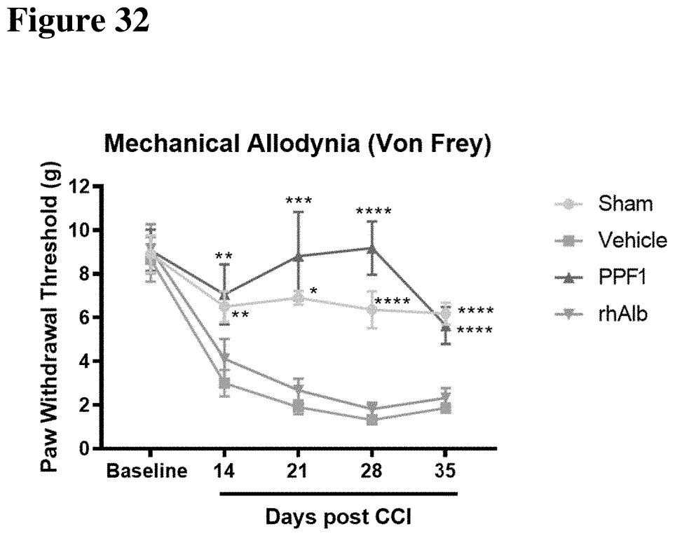

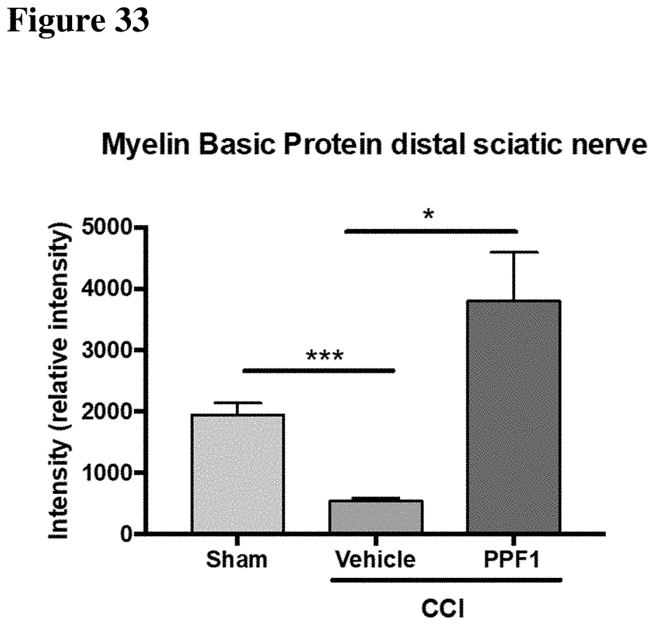

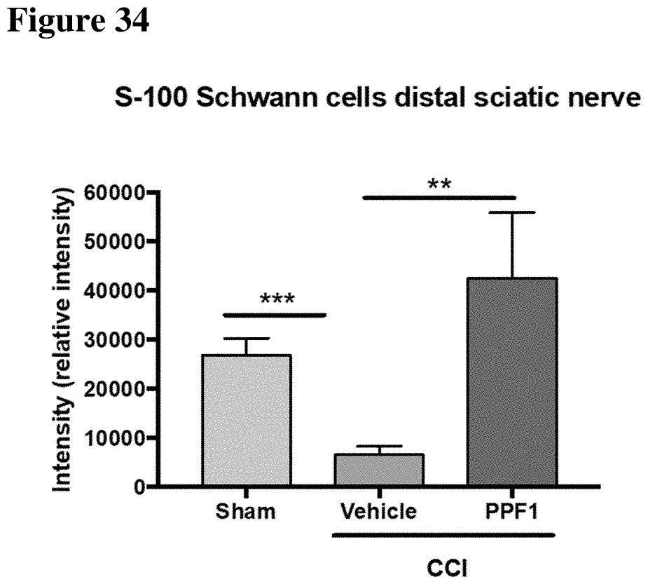

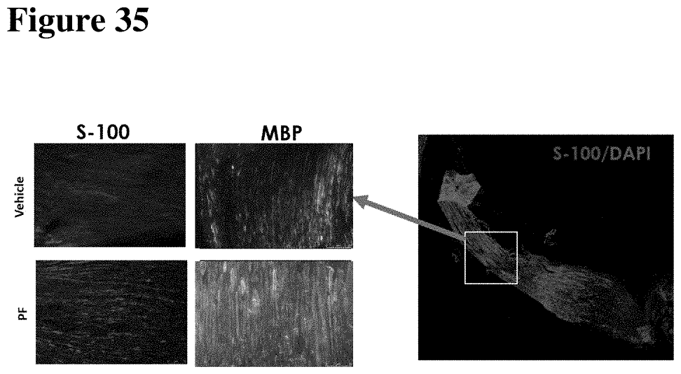

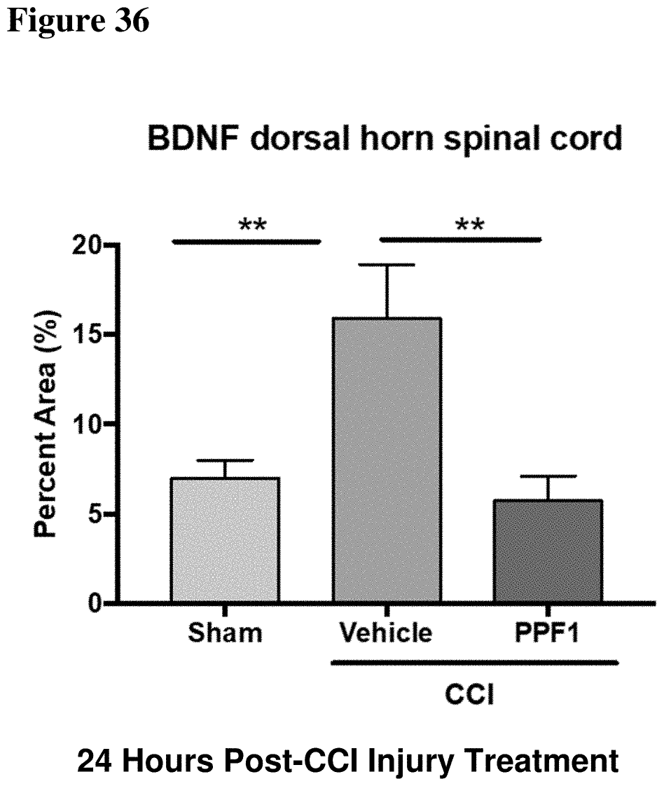

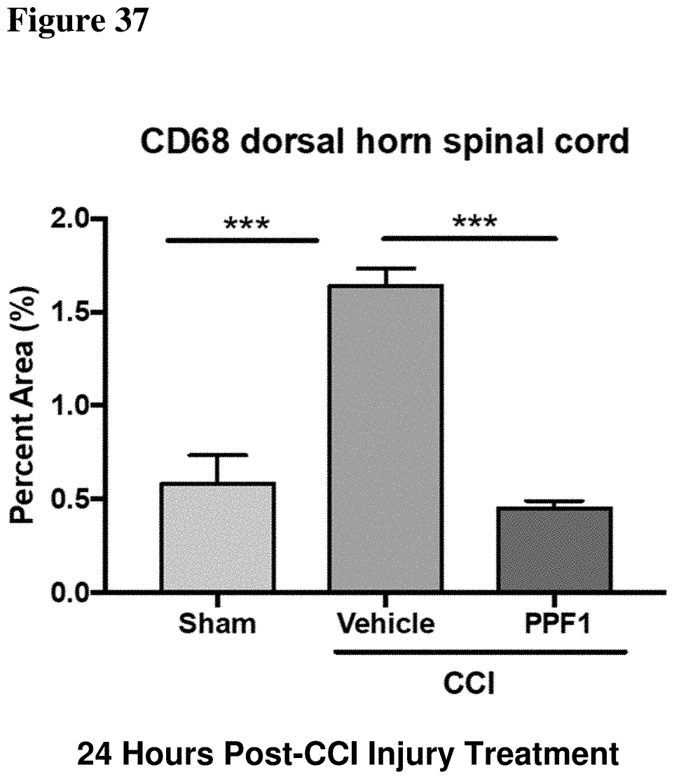

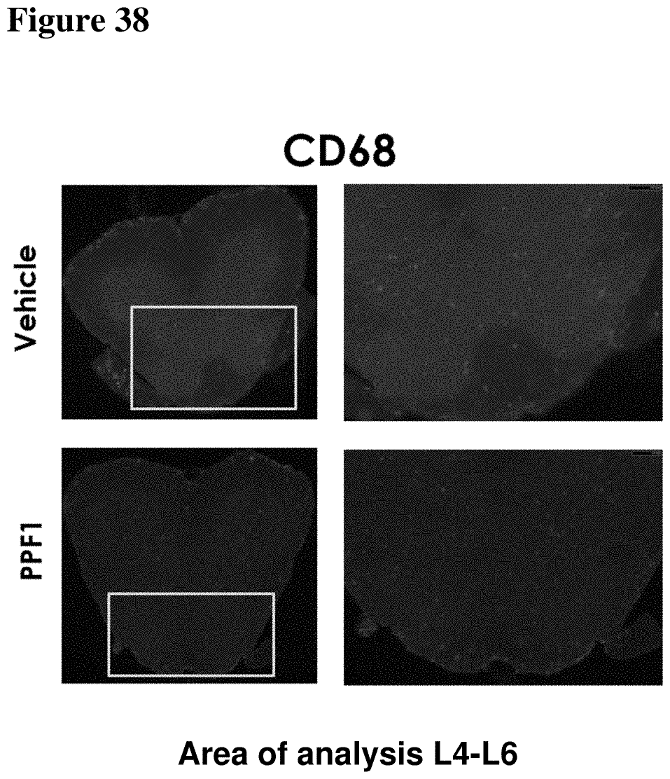

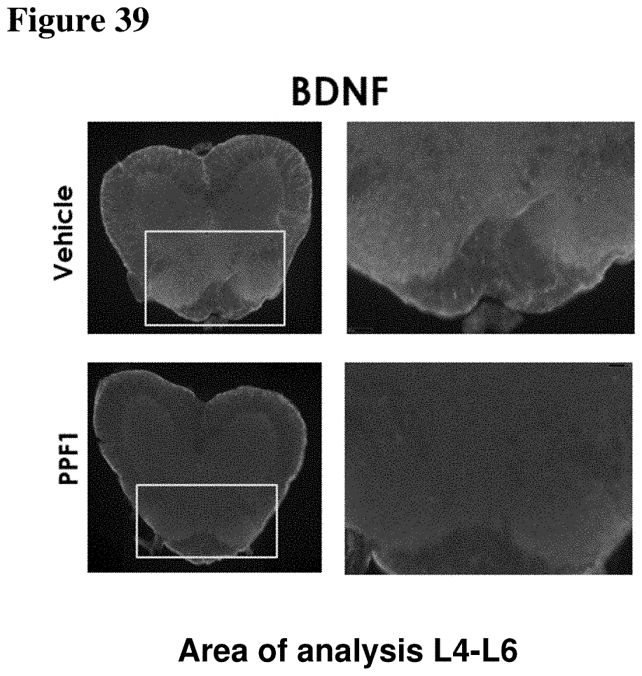

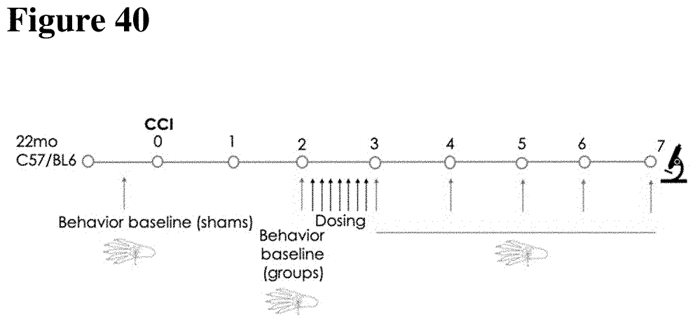

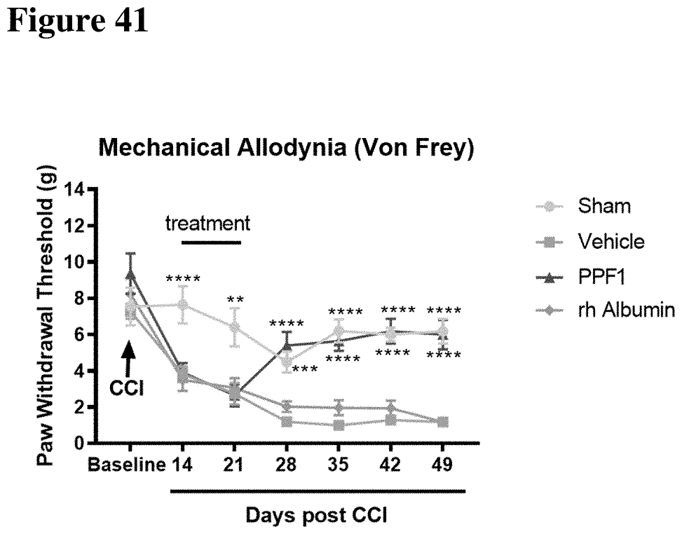

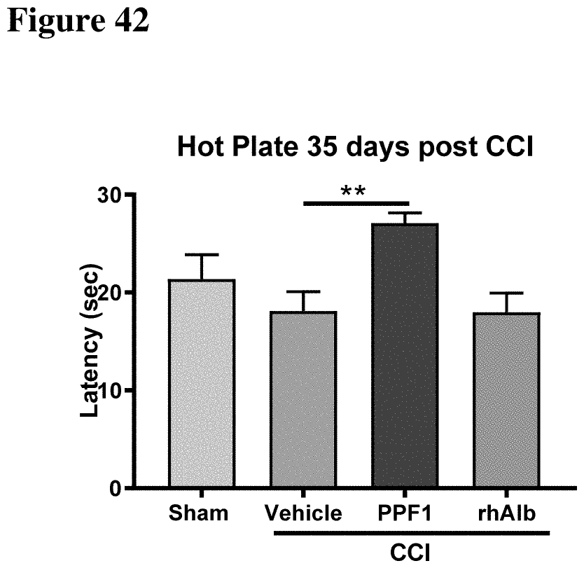

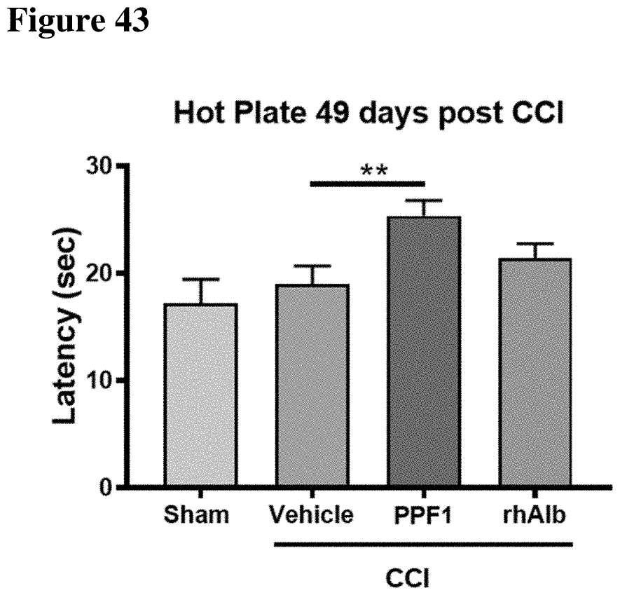

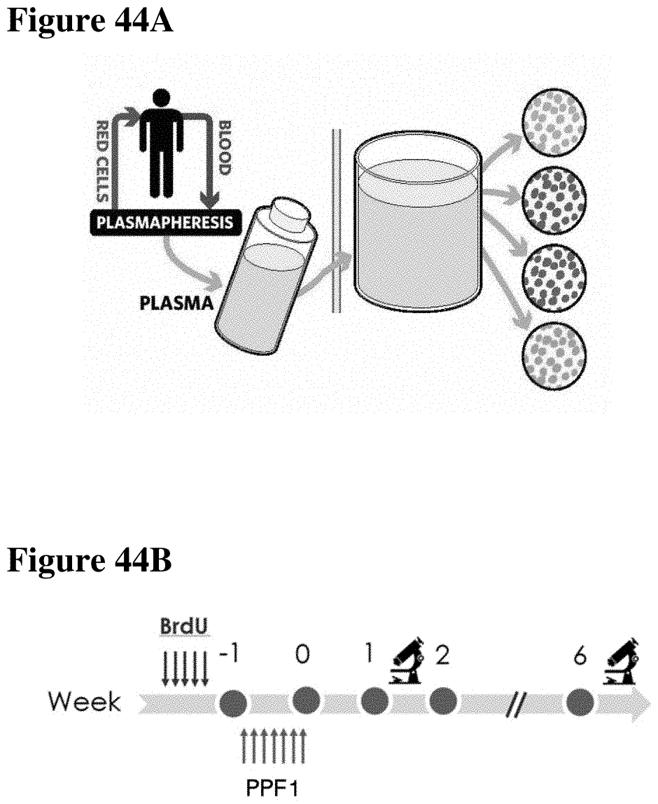

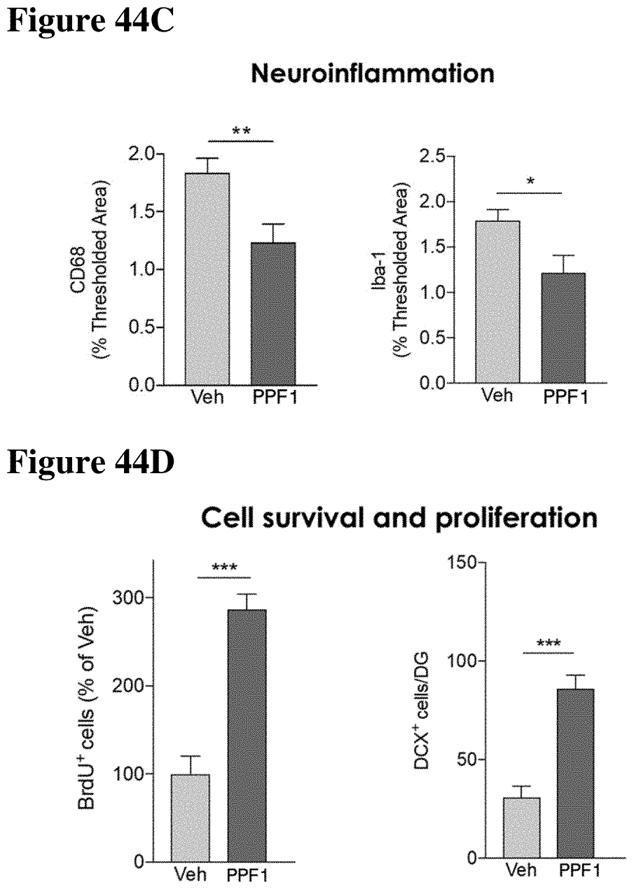

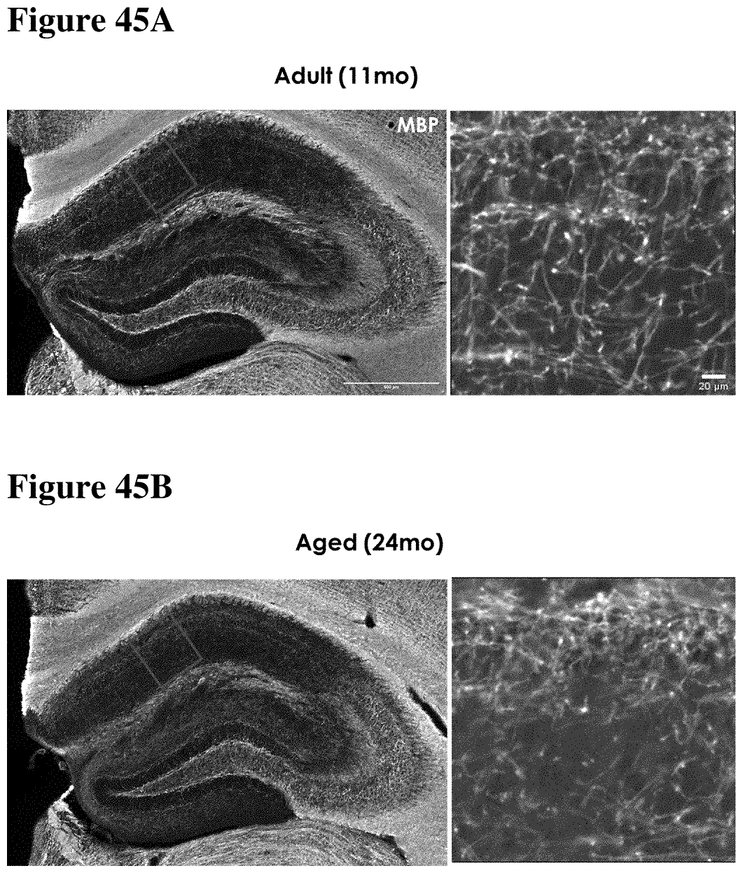

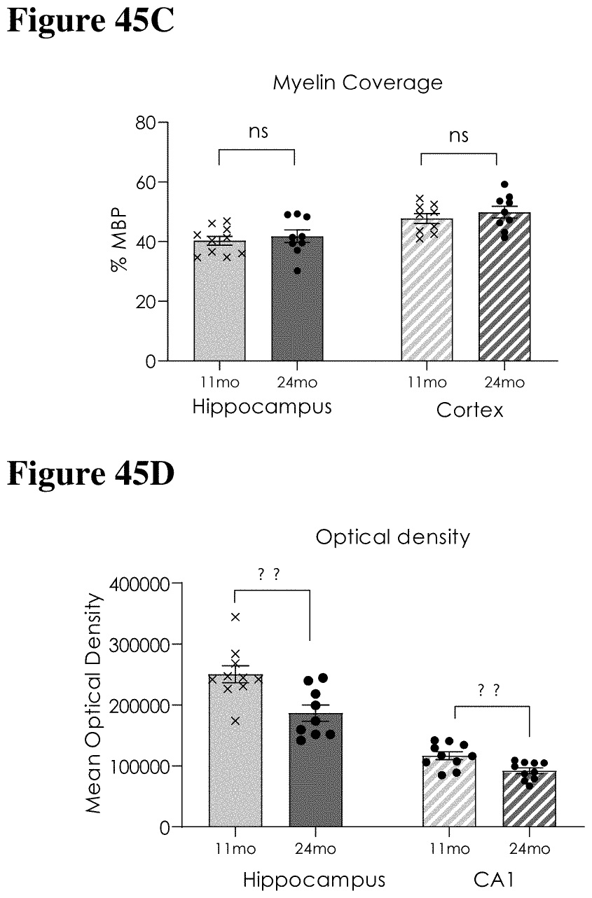

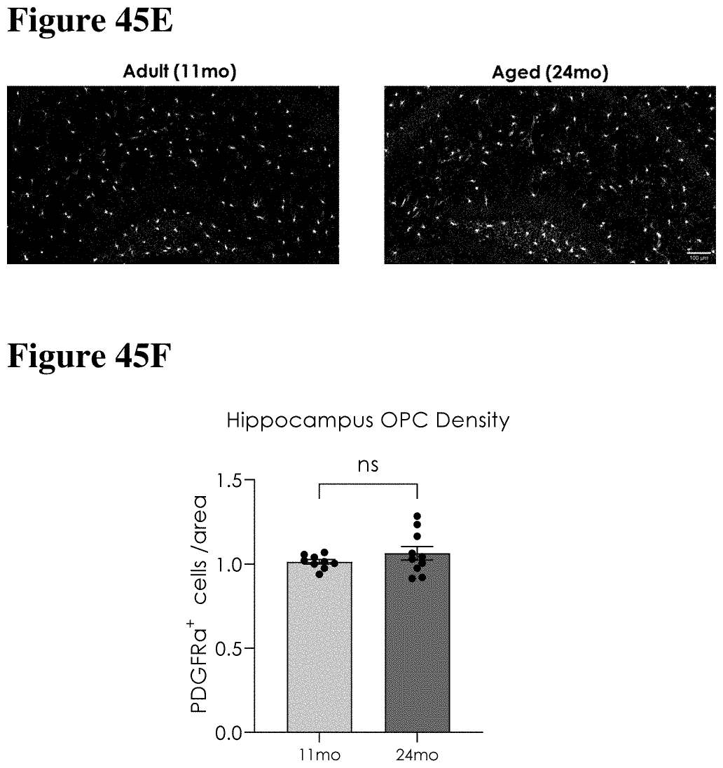

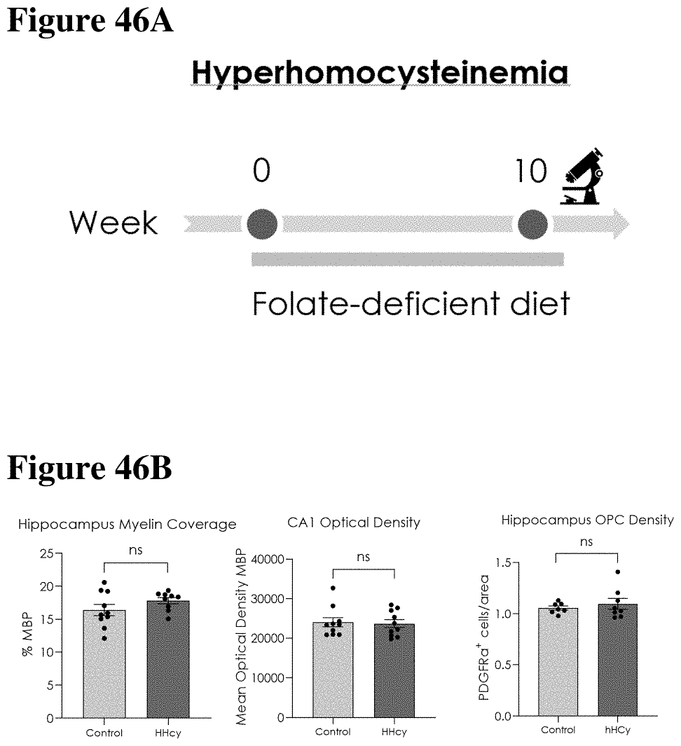

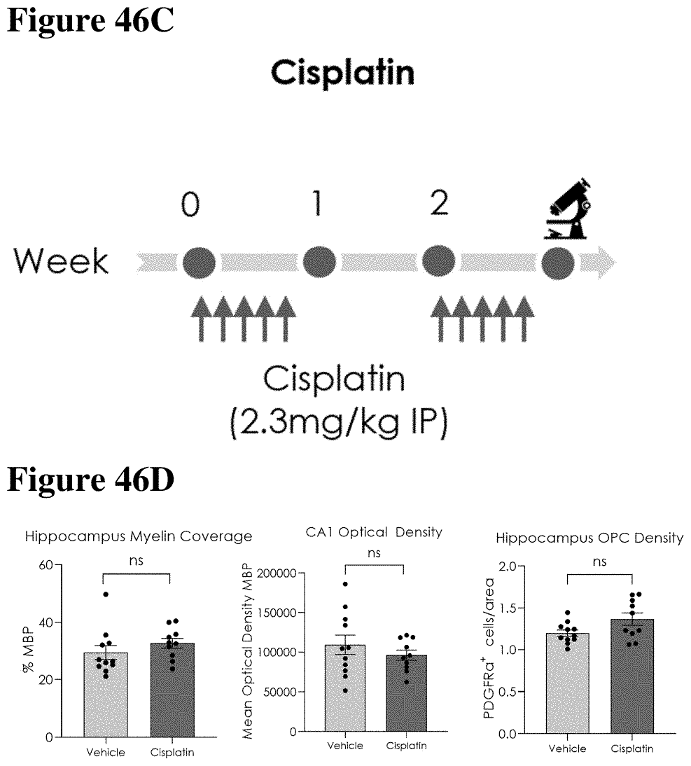

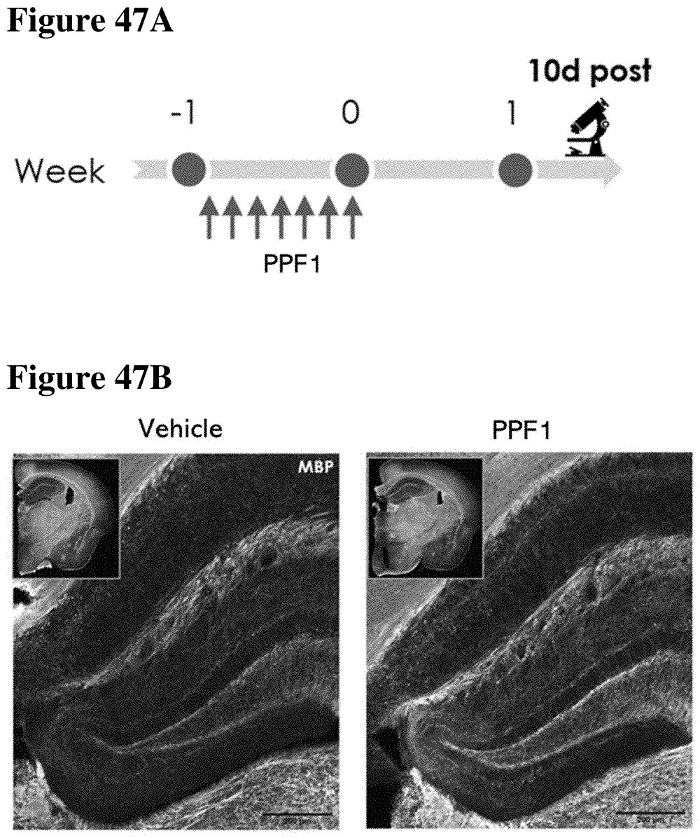

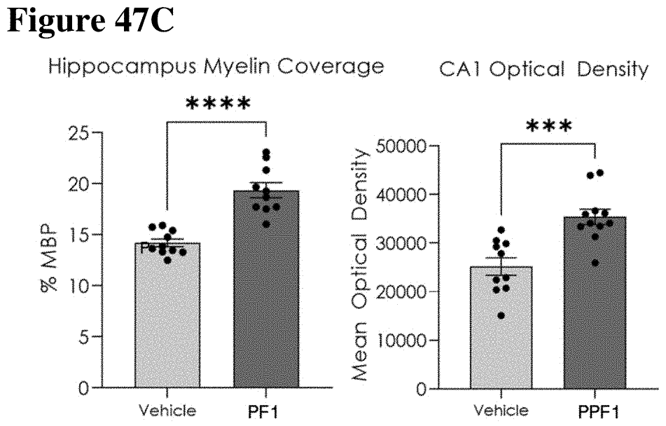

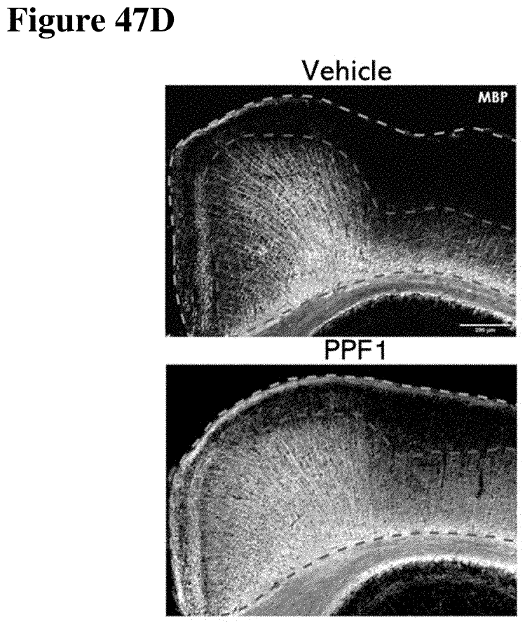

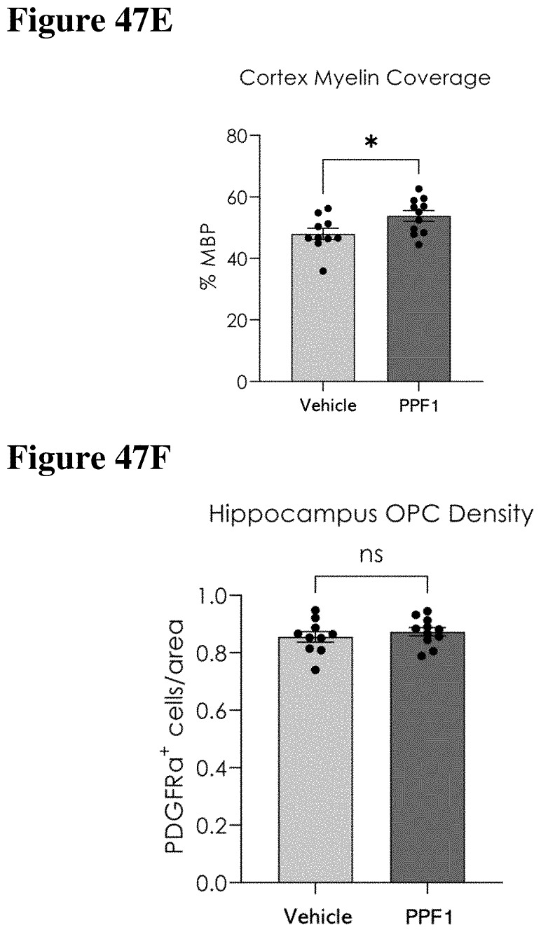

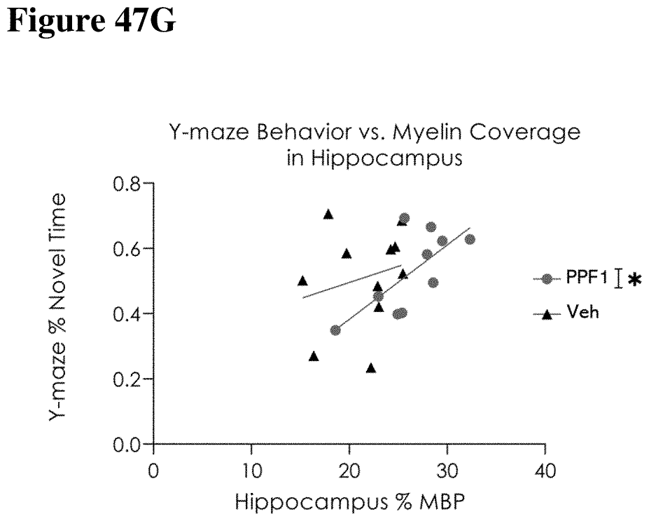

FIG. 22D shows the quantification of neurosphere size as percent of vehicle from cortices from E14-15 mouse embryos suspended in neural basal media supplemented with B27 and 2 mM Glutamax (vehicle), PPF1 (10% of a 5% stock solution), or HAS1 (10% of a 5% stock solution).

FIG. 23 shows the quantification of the number of neurospheres staining positive for Sox2, which were treated with control vehicle (neural basal media supplemented with B27 and 2 mM Glutamax), PPF1 (10% of a 5% stock solution), or HAS1 (10% of a 5% stock solution). Sox2 staining is an indicator of a neurosphere's potential for neurogenesis.

FIG. 24 depicts a chronic constrictive injury (CCI) experiment. Twenty-three-month-old wild type mice were administered a CCI or sham surgery via ligation 24 hours prior to administration of a 7-consecutive-day pulse dosing regimen of either PPF1, Gabapentin, recombinant human albumin (rhAlb) or vehicle control. Behavior was assessed during weeks two through five, and tissue collection for histology occurred during week five.

FIG. 25 is a representation depicting the location of the CCI administered to twenty-two-month-old wild type mice. The ligation was administered on the sciatic nerve as indicated by the figure. The figure was adapted from Suter M R, et al., Anesthesiology Res and Practice, (2011), which is incorporated herein by reference in its entirety.

FIG. 26 reports data from a mechanical von Frey allodynia test in wild-type mice treated with CCI or sham surgery described in FIG. 24. Useful for the analysis of pain behavior, the hind paw enervated by the subject sciatic nerve, was administered with von Frey filament stimulation. The pressure at which the mouse withdrew its hind paw was measured and plotted in The figure shows that mice treated with PPF1 after CCI exhibited significantly less pain (could withstand more pressure) than those treated with vehicle control after CCI. And sham operations treated with vehicle also exhibited significantly less pain that those treated with vehicle control after CCI. This shows that PPF1 has a positive effect on mechanical nociception deficits.

FIG. 27 reports data from hippocampal histology performed on the wild type mice described in FIG. 24. Neurogenesis was measured using the doublecortin (DCX) marker. Mice given CCI who were treated with PPF1 had significantly more neurogenesis in the hippocampus than those who received vehicle. Mice given sham operations plus vehicle trended towards greater neurogenesis than mice given CCI and vehicle post-surgery. Thus, PPF1 exhibited the ability to restore neurogenesis after chronic nerve injury.

FIG. 28 reports data from hippocampal histology performed on the wild type mice described in FIG. 24. CD68 expression was quantified, and mice given a CCI plus vehicle expressed a significantly greater number of CD68 positive cells in the hippocampus than those given a CCI plus PPF1. A similar degree of difference was observed between mice given a CCI plus vehicle and those given a sham surgery plus vehicle. This shows that PPF1 can help to block neuroinflammation resulting from chronic nerve injury.

FIG. 29 reports data from a mechanical von Frey allodynia test in twenty-two-month-old C57BL/6J mice which received CCI or sham surgery and tested in the timeline as described in FIG. 24. The pressure at which the mouse withdrew its hind paw was assessed and represented in FIG. 29 as weeks post CCI or sham surgery. The figure illustrates that mice administered PPF1 following CCI surgery had significantly increased tolerance to mechanical nociception at all assessed timepoints than those treated with vehicle after CCI. Conversely, mice administered Gabapentin only show significant improvement in mechanical nociception at 2 weeks following CCI surgery and are similar to vehicle treated mice at all other timepoints. Sham surgery mice show significantly increased response to mechanical nociception at 3 and 5 weeks following surgical manipulation. Together, these data illustrate that PPF1 ameliorates peripheral pain for a greater amount of time than that of standard of care treatments (Gabapentin).

FIG. 30 reports data from a hot plate test on twenty-two-month-old wild-type mice which received CCI or sham surgery and tested in the timeline as described in FIG. 24. This assay was performed as described by Woolfe and Macdonald. (Woolfe G. and Macdonald A D, J. Pharmacol. Exp. Ther. 80:300-07 (1944), which is incorporated by reference herein in its entirety). The hot plate is set to a temperature of 55° C. Mice are acclimated to being placed inside a clear cylinder for 30 minutes. The cylinder is placed upon the hot plate and a timer started. When nocifensive behaviors (e.g. hind paw licking or jumping) are first observed, the time is recorded as latency. FIG. 30 illustrates hot plate nocifensive latency 5 weeks after CCI or sham surgery. PPF1 treatment are significantly less sensitive to hot plate stimuli compared to mice given CCI plus vehicle control, indicating a rescue effect by PPF1. Whereas, standard of care effects (Gabapentin) are similar to that of vehicle.

FIG. 31 reports data from a hot plate test on wild-type mice which received CCI or sham surgery and tested in the timeline as described in FIG. 24. FIG. 31 illustrates hot plate nocifensive latency 5 weeks after CCI or sham surgery. PPF1 treatment and rhALB are significantly less sensitive to hot plate stimuli compared to mice given CCI plus vehicle control.

FIG. 32 reports data from a mechanical von Frey allodynia test in C57BL/6J mice which received CCI or sham surgery and tested in the timeline as described in FIG. 24. FIG. 32 illustrates that mice administered PPF1 following CCI surgery had significantly increased tolerance to mechanical nociception at all assessed timepoints than those treated with vehicle after CCI. Conversely, mice administered rhALB have similar response to mechanical allodynia to vehicle treated mice at all timepoints.

FIG. 33 reports data from sciatic nerve histological analysis (approximately 1000 μm distal from the last ligature) of myelin basic protein (MBP) expression in C57BL/6J mice which received CCI or sham surgery and analyzed following tissue collection after day 35 as described in FIG. 24. FIG. 33 illustrates that mice administered PPF1 following CCI surgery had significantly increased MBP intensity, indicative of increased myelin expression as compared to vehicle treated animals. Sham mice also express increased MBP as compared to CCI injured vehicle mice.

FIG. 34 reports data from sciatic nerve histological analysis (approximately 1000 um distal from the last ligature) of S-100 protein (expressed by Schwann cells) in C57BL/6J mice which received CCI or sham surgery and analyzed following tissue collection after day 35 as described in FIG. 24. FIG. 34 illustrates that mice administered PPF1 following CCI surgery had significantly increased S-100 intensity, indicative of increased Schwann cells (which are myelin producing cells in peripheral nerves) as compared to vehicle treated animals. Sham mice also express increased S-100 as compared to CCI injured vehicle mice.

FIG. 35 are images selected from sciatic nerve histological analysis which identify the location used for quantification in FIGS. 33 and 34 (approximately 1000 μm distal from the last ligature) and representative intensities of S-100 protein (expressed by Schwann cells) and Myelin Basic Protein in C57BL/6J mice which received CCI surgery and were treated with either vehicle or PPF1 and used for qualitative analysis of sciatic nerve tissue after day 35 as described in FIG. 24.

FIG. 36 reports data from spinal cord histological analysis (performed on spinal cord tissue collected from the lumbar section L4-L6) of C57BL/6J mice which received CCI or sham surgery and analyzed following tissue collection after day 35 as described in FIG. 24. FIG. 36 illustrates that mice administered PPF1 following CCI surgery had significantly decreased BDNF intensity within the dorsal horns of the spinal cord, indicative of decreased activation of microglia within the spinal cord. As BDNF is a pro-inflammatory cytokine released by activated microglia, these findings suggest that PPF1 is decreasing a fundamental regulator of pain states within the spinal cord, normalizing the level to that of sham (non-CCI injured) mice.

FIG. 37 reports data from spinal cord histological analysis (performed on spinal cord tissue collected from the lumbar section L4-L6) of C57BL/6J mice which received CCI or sham surgery and analyzed following tissue collection after day 35 as described in FIG. 24. FIG. 37 illustrates that mice administered PPF1 following CCI surgery had significantly decreased CD68 intensity within the dorsal horns of the spinal cord, indicative of decreased activation of microglia within the spinal cord. As CD68 protein is expressed by activated microglia, this suggests that PPF1 is decreasing the activation of the fundamental cell type responsible for induction of pain states within the spinal cord, normalizing the level to that of sham (non-CCI injured) mice. Data presented in FIG. 36 and FIG. 37 indicate that PPF1 is centrally regulating the pain state resulting from sciatic nerve injury and ameliorating or preventing the establishment of pain signaling between the peripheral nerves and the brain, also described as central sensitization.

FIG. 38 are images selected from spinal cord histological analysis which identify the location of dorsal horns used for quantification in FIG. 37 (performed on spinal cord tissue collected from the lumbar section L4-L6) and representative intensities of CD68 protein (expressed by activated microglia) in C57BL/6J mice which received CCI surgery and were treated with either vehicle or PPF1 and used for qualitative analysis of spinal cord tissue after day 35 as described in FIG. 24.

FIG. 39 are images selected from spinal cord histological analysis which identify the location of dorsal horns used for quantification in FIG. 36 (performed on spinal cord tissue collected from the lumbar section L4-L6) and representative intensities of BDNF protein (a cytokine released by activated microglia) in C57BL/6J mice which received CCI surgery and were treated with either vehicle or PPF1 and used for qualitative analysis of spinal cord tissue after day 35 as described in FIG. 24.

FIG. 40 depicts a chronic constrictive injury (CCI) experiment. Twenty-two-month-old wild type mice were administered a CCI or sham surgery via ligation 2 weeks prior to administration of a 7-consecutive-day pulse dosing regimen of either PPF1, rhALB or vehicle control. Behavior was assessed weekly during weeks two through seven, and tissue collection for histology occurred during week seven.

FIG. 41 reports data from a mechanical von Frey allodynia test in C57BL/6J mice which received CCI or sham surgery and tested in the timeline as described in FIG. 40. FIG. 41 illustrates that mice administered PPF1 two weeks following CCI surgery had significantly increased tolerance to mechanical nociception beginning at a timepoint one week following the cessation of PPF1 treatment which was maintained throughout the duration of the study. Findings in FIG. 41 suggest that PPF1 treatment initiates processes which reduce sensitivity to mechanical allodynia in a longitudinal fashion, as improved tolerance isn't evidenced until a week following treatment (in contrast with therapies which exclusively provide benefit during treatment, such as opioid analgesics) and is sustained for at least 28 days. Conversely, mice administered rhALB have similar response to mechanical allodynia to vehicle treated mice at all timepoints.

FIG. 42 reports data from a hot plate test on wild-type mice which received CCI or sham surgery and tested in the timeline as described in FIG. 40. FIG. 42 illustrates hot plate nocifensive latency 5 weeks after CCI or sham surgery. PPF1 treatment is significantly less sensitive to hot plate stimuli compared to mice given CCI plus vehicle control.

FIG. 43 reports data from a hot plate test on wild-type mice which received CCI or sham surgery and tested in the timeline as described in FIG. 40. FIG. 43 illustrates hot plate nocifensive latency 7 weeks after CCI or sham surgery. PPF1 treatment is significantly less sensitive to hot plate stimuli compared to mice given CCI plus vehicle control.

FIGS. 44A-44D show that plasma fraction treatment decreases neuroinflammation and enhances neurogenesis. FIG. 44A shows a schematic of a fractionation process for plasma fractions. FIG. 44B shows a schematic of study design with 22 to 24-month-old wildtype male mice dosed with PPF1 and analyzed 10 days (CD68/Iba-1) or 6 weeks later (BrdU/DCX). FIG. 44C shows quantification of CD68 and Iba-1 immunoreactivity in the hippocampus, indicating a decrease in microgliosis with PPF1 treatment. FIG. 44D is a quantification of BrdU and DCX immunoreactivity in the hippocampus, showing that PPF1 treatment improves cell survival and neurogenesis. All data shown are mean±SEM; *p<0.05, **p<0.01, ***p<0.001. Veh: Vehicle.

FIGS. 45A-45F show age-related decrease of myelin in the hippocampus. FIGS. 45A-45B show representative hippocampal images at 11 mo and 24 mo. The box highlights the CA1 ROI shown in image to the right. FIG. 45C shows that myelin coverage in the hippocampus and cortex did not change from 11 mo to 24 mo. FIG. 45D shows that the mean optical density of MBP signal is significantly increased in the hippocampus and the CA1 in the 11 mo mice compared to 24 mo mice. FIG. 45E provides representative images of PDGFRa⁺ cells in the hippocampus of 11 mo and 24 mo mice. FIG. 45F shows that PDGFRa⁺ cell density in the hippocampus did not change with age. Data shown are mean±SEM; Mann-Whitney Test. **p<0.003. Scale bar=500 μm, 100 μm, 20 μm.

FIGS. 46A-46D show that Hhcy and cisplatin models do not show deficits in myelin content. FIG. 46A shows a protocol for inducing Hhcy in 12-week-old mice via folate-deficient feed for 10 weeks. No difference was found in percent area coverage of myelin or MBP optical density in the hippocampus or OPC density as measured by PDGFRa in the hippocampus (FIG. 46B). FIG. 46C shows a schematic of a protocol for inducing cognitive impairment in 7-month-old mice by IP dosing with 2.3 mg/kg cisplatin. FIG. 46D shows that no difference was found in percent area coverage of myelin, optical density in the hippocampus, or OPC density as measured by PDGFRa in the hippocampus. All data shown are mean±SEM.

FIGS. 47A-47G show that aged mice treated with PPF1 show increased myelin content in the hippocampus and cortex. FIG. 47A shows a schematic of the experimental protocol. FIG. 47B shows that hippocampus ROI (inset) and representative dentate gyrus images show an increase in MBP expression in PPF1-treated mice. FIG. 47C shows that percent area of myelin coverage and optical density of MBP in the hippocampus and CA1 increased with PPF1 treatment. FIG. 47D shows representative images of MBP expression in the cortex (inner dotted line) and ROI (outer dotted line). FIG. 47E shows that an increased MBP expression is observed in the cortex with PPF1 treatment. FIG. 47F shows that no difference in PDGFRa⁺ OPC density was observed in the hippocampus. FIG. 47G shows that a significant correlation was observed between MBP expression with Y-maze performance (percent time in the novel arm) with PPF1 treatment. Spearman Correlation test. R=0.7182, *p=0.0162; Mann-Whitney Test. ****p<0.0001, ***p<0.0002, *p=0.03. Scare bar=200 μm.

### DETAILED DESCRIPTION OF THE INVENTION

### 1. Introduction

The present invention relates to the identification and discovery of methods and compositions for the treatment and/or prevention of cognitive impairment, including age-associated dementia and neurodegenerative disease. Described herein are methods and compositions for the treatment of subjects suffering from such disorders, which are aspects of the present invention. The methods and compositions described herein are useful in: preventing cognitive impairment, age-associated dementia, and neurodegenerative disease; ameliorating the symptoms of cognitive impairment, age-associated dementia, and neurodegenerative disease; slowing progression of aging-associated cognitive impairment, age-associated dementia, and neurodegenerative disease; and/or reversing the progression of aging-associated cognitive impairment, age-associated dementia, and neurodegenerative disease.

The present invention also relates to the identification and discovery of methods and compositions for the treatment of unwanted conditions associated with postoperative recovery, and for improving such recovery. By “improving such recovery,” it is meant that a subject's postoperative recovery may be accelerated, i.e. the subject may become mobile or be discharged from in-patient care in less time than it would take without the intervention of the embodiments of the present invention. By “unwanted conditions,” it is meant a condition or symptom such as, by way of example and not limitation, pain, cardiopulmonary issues, infections, thromboembolic issues, inflammation, and delayed wound healing. Described herein are methods and compositions for the treatment of subjects suffering from unwanted conditions associated with postoperative recovery, and for improving such recovery, which are aspects of the present invention. Also described herein are dosing regimens which trigger improvement in subjects suffering from unwanted conditions associated with postoperative recovery, and for improving such recovery. The methods and compositions described herein are useful in: preventing complications from postoperative recovery; ameliorating the symptoms of preventing complications from postoperative recovery; and accelerating postoperative recovery. The methods and compositions of the invention may be utilized or administered preoperatively (before surgery); perioperatively (during surgery); or postoperatively (after surgery).

Another aspect of the invention is for treating chronic pain/neuropathy more generally, and not exclusively chronic pain/neuropathy associated with postoperative recovery. The methods and compositions of the invention described herein can be used to treat chronic pain and neuropathy. By “treating chronic pain and neuropathy” it is meant that the degree of chronic pain experienced by the subject to whom is administered the compositions of the invention is lessened, slightly, moderately, or significantly as assessed by subjective or objective means. Such means may include self- or medical professional-administered tests such as, by way of example and not limitation: X-ray; MRI, CT scans; patient rating or description of the pain; range of motion; reflexes, muscle strength; sensitivity (e.g. how long it takes for the subject to remove a limb that is subjected to pressure or other stimulus); blood tests for inflammatory markers; electromyography (EMG); and nerve conduction velocity).

A further aspect of the invention is for using blood products, such as blood plasma and blood plasma fractions, to restore myelin levels in conditions associated with myelin degeneration, for example, aging-associated neurocognitive and neurodegenerative disorders or a myelopathy associated with post-operative recovery. Restoring myelin levels in such diseases improves nerve conductance.

Accordingly, certain embodiments of this invention provide a method of restoring myelin levels and/or improving nerve conductance, comprising administering an effective amount of a Plasma Fraction to a subject diagnosed with a condition associated with myelin degeneration. A condition associated with myelin degeneration can be neurodegenerative and/or neuroinflammatory condition, such as an aging-associated neurocognitive, neurodegenerative, and/or neuroinflammatory condition. In some cases, a condition associated with myelin degeneration is a myelopathy associated with postoperative recovery.

In some cases, a Plasma Fraction is a Plasma Protein Fraction (PPF), which can be a commercially available PPF. A PPF can have a total protein content that consists of at least 83 percent but less than 95 percent albumin and no more than 17 percent globulins. A PPF can also have no more than 1 percent gamma globulins.

The Plasma Fraction can be derived from plasma obtained from a pool of young individuals, for example, a pool of humans between the ages of 0 and 40, e.g., 0, 1, 5, 10, 15, 20, 25, 30, 35, or 40 years old.

The Plasma Fraction can be produced from a mammalian blood product, particularly, a human blood product.

The subject can be a mammal, particularly, a human.

An implementation of the invention includes using blood plasma fractions as treatment, such as one or more fractions or effluents obtained from blood fractionation processes, e.g., like the Cohn fractionation process described below. An embodiment of the invention includes using Plasma Fraction (a solution comprised of normal human albumin, alpha and beta globulins, gamma globulin, and other proteins either individually or as complexes, hereinafter referred to as “Plasma Fraction”). Another embodiment of the invention includes using Plasma Protein Fraction (PPF) as treatment. Another embodiment of the invention includes using Human Albumin Solution (HAS) fraction as treatment. Yet another embodiment includes using effluents from blood fractionation processes such as Effluent I or Effluent II/III described below. An additional embodiment includes a blood plasma fraction from which substantially all the clotting factors have been removed in order to retain efficacy while reducing the risk of thromboses (for example, see U.S. Patent Application Nos. 62/236,710 and 63/376,529, which are incorporated by reference in their entirety herein).

Before describing the present invention in detail, it is to be understood that this invention is not limited to a particular method or composition described, as such may, of course, vary. It is also understood that the terminology used herein is for the purpose of describing particular embodiments only, and is not intended to be limiting, since the scope of the present invention will be limited only by the appended claims.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

It is noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein have discrete components and features which may be readily separated from or combined with the features of any of the other several embodiments without departing from the scope or the spirit of the present invention. Any recited method can be carried out in the order of events recited or in any other order which is logically possible.

### 2. Definitions

Unless otherwise defined, all technical and scientific terms used herein have the same meaning as commonly understood by one having ordinary skill in the art to which the invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a,” “an,” and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those having skill in the art, and so forth.

In describing methods of the present invention, the terms “host”, “subject”, “individual” and “patient” are used interchangeably and refer to any mammal in need of such treatment according to the disclosed methods. Such mammals include, e.g., humans, ovines, bovines, equines, porcines, canines, felines, non-human primate, mice, and rats. In certain embodiments, the subject is a non-human mammal. In some embodiments, the subject is a farm animal. In other embodiments, the subject is a pet. In some embodiments, the subject is mammalian. In certain instances, the subject is human. Other subjects can include domestic pets (e.g., dogs and cats), livestock (e.g., cows, pigs, goats, horses, and the like), rodents (e.g., mice, guinea pigs, and rats, e.g., as in animal models of disease), as well as non-human primates (e.g., chimpanzees, and monkeys). As such, subjects of the invention, include but are not limited to mammals, e.g., humans and other primates, such as chimpanzees and other apes and monkey species; and the like, where in certain embodiments the subject are humans. The term subject is also meant to include a person or organism of any age, weight or other physical characteristic, where the subjects may be an adult, a child, an infant or a newborn.

By a “young” or “young individual” it is meant an individual that is of chronological age of 40 years old or younger, e.g., 35 years old or younger, including 30 years old or younger, e.g., 25 years old or younger or 22 years old or younger. In some instances, the individual that serves as the source of the young plasma-comprising blood product is one that is 10 years old or younger, e.g., 5 years old or younger, including 1-year-old or younger. In some instances, the subject is a newborn and the source of the plasma product is the umbilical cord, where the plasma product is harvested from the umbilical cord of the newborn. As such, “young” and “young individual” may refer to a subject that is between the ages of 0 and 40, e.g., 0, 1, 5, 10, 15, 20, 25, 30, 35, or 40 years old. In other instances, “young” and “young individual” may refer to a biological (as opposed to chronological) age such as an individual who has not exhibited the levels of inflammatory cytokines in the plasma exhibited in comparatively older individuals. Conversely, these “young” and “young individual” may refer to a biological (as opposed to chronological) age such as an individual who exhibits greater levels of anti-inflammatory cytokines in the plasma compared to levels in comparatively older individuals. By way of example, and not limitation, the inflammatory cytokine is Eotaxin, and the fold difference between a young subject or young individual and older individuals is at least 1.5-fold. Similarly, the fold difference between older and younger individuals in other inflammatory cytokines may be used to refer to a biological age. (See U.S. patent application Ser. No. 13/575,437 which is herein incorporated by reference). Usually, the individual is healthy, e.g., the individual has no hematological malignancy or autoimmune disease at the time of harvest.

By “an individual suffering from or at risk of suffering from an aging-associated cognitive impairment” is meant an individual that is about more than 50% through its expected lifespan, such as more than 60%, e.g., more than 70%, such as more than 75%, 80%, 85%, 90%, 95% or even 99% through its expected lifespan. The age of the individual will depend on the species in question. Thus, this percentage is based on the predicted life-expectancy of the species in question. For example, in humans, such an individual is 50 year old or older, e.g., 60 years old or older, 70 years old or older, 80 years old or older, 90 years old or older, and usually no older than 100 years old, such as 90 years old, i.e., between the ages of about 50 and 100, e.g., 50 . . . 55 . . . 60 . . . 65 . . . 70 . . . 75 . . . 80 . . . 85 . . . 90 . . . 95 . . . 100 years old or older, or any age between 50-1000, that suffers from an aging-associated condition as further described below, e.g., cognitive impairment associated with the natural aging process; an individual that is about 50 years old or older, e.g., 60 years old or older, 70 years old or older, 80 years old or older, 90 years old or older, and usually no older than 100 years old, i.e., between the ages of about 50 and 100, e.g., 50 . . . 55 . . . 60 . . . 65 . . . 70 . . . 75 . . . 80 . . . 85 . . . 90 . . . 95 . . . 100 years old, that has not yet begun to show symptoms of an aging-associated condition e.g., cognitive impairment; an individual of any age that is suffering from a cognitive impairment due to an aging-associated disease, as described further below, and an individual of any age that has been diagnosed with an aging-associated disease that is typically accompanied by cognitive impairment, where the individual has not yet begun to show symptoms of cognitive impairment. The corresponding ages for non-human subjects are known and are intended to apply herein.

As used herein, in the appropriate context, “treatment” refers to any of (i) the prevention of the disease or disorder, or (ii) the reduction or elimination of symptoms of the disease or disorder. Treatment may be effected prophylactically (prior to the onset of disease) or therapeutically (following the onset of the disease). The effect may be prophylactic in terms of completely or partially preventing a disease or symptom thereof and/or may be therapeutic in terms of a partial or complete cure for a disease and/or adverse effect attributable to the disease. Thus, the term “treatment” as used herein covers any treatment of an aging-related disease or disorder in a mammal, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease. Treatment may result in a variety of different physical manifestations, e.g., modulation in gene expression, rejuvenation of tissue or organs, etc. The therapeutic agent may be administered before, during or after the onset of disease. The treatment of ongoing disease, where the treatment stabilizes or reduces the undesirable clinical symptoms of the patient, is of particular interest. Such treatment may be performed prior to complete loss of function in the affected tissues. The subject therapy may be administered during the symptomatic stage of the disease, and in some cases after the symptomatic stage of the disease.

As used herein, in the appropriate context, “treatment” also refers to any of (i) the prevention of the disease or disorder, or (ii) the reduction or elimination of symptoms of the disease or disorder. Treatment may be effected prophylactically (prior to the onset of disease) or therapeutically (following the onset of the disease). The effect may be prophylactic in terms of completely or partially preventing a disease or symptom thereof and/or may be therapeutic in terms of a partial or complete cure for a disease and/or adverse effect attributable to the disease. Thus, the term “treatment” as used herein covers any treatment of a condition associated with postoperative recovery in a mammal and includes: (a) preventing the condition from occurring in a subject; (b) inhibiting the condition, i.e., arresting its occurrence; or (c) relieving the condition, i.e., causing regression of the condition. Treatment may result in a variety of different physical manifestations, e.g., modulation in gene expression, rejuvenation of tissue or organs, decreasing inflammation, etc. The therapeutic agent may be administered before, during or after the onset of the condition. The subject therapy may be administered during the symptomatic stage of the condition, and in some cases after the symptomatic stage of the condition.

In some embodiments, the aging-associated condition that is treated is an aging-associated impairment in cognitive ability in an individual. By cognitive ability, or “cognition,” it is meant the mental processes that include attention and concentration, learning complex tasks and concepts, memory (acquiring, retaining, and retrieving new information in the short and/or long term), information processing (dealing with information gathered by the five senses), visuospatial function (visual perception, depth perception, using mental imagery, copying drawings, constructing objects or shapes), producing and understanding language, verbal fluency (word-finding), solving problems, making decisions, and executive functions (planning and prioritizing). By “cognitive decline”, it is meant a progressive decrease in one or more of these abilities, e.g., a decline in memory, language, thinking, judgment, etc. By “an impairment in cognitive ability” and “cognitive impairment”, it is meant a reduction in cognitive ability relative to a healthy individual, e.g., an age-matched healthy individual, or relative to the ability of the individual at an earlier point in time, e.g., 2 weeks, 1 month, 2 months, 3 months, 6 months, 1 year, 2 years, 5 years, or 10 years or more previously. By “aging-associated cognitive impairment,” it is meant an impairment in cognitive ability that is typically associated with aging, including, for example, cognitive impairment associated with the natural aging process, e.g., mild cognitive impairment (M.C.I.); and cognitive impairment associated with an aging-associated disorder, that is, a disorder that is seen with increasing frequency with increasing senescence, e.g., a neurodegenerative condition such as Alzheimer's disease, Parkinson's disease, frontotemporal dementia, Huntington disease, amyotrophic lateral sclerosis, multiple sclerosis, glaucoma, myotonic dystrophy, vascular dementia, and the like.

Blood Products Comprising Plasma Components. In practicing the subject methods, a blood product comprising plasma components is administered to an individual in need thereof, e.g., an individual suffering or at risk of suffering from a cognitive impairment and/or age-related dementia or a postoperative condition. As such, methods according to embodiments of the invention including administering a blood product comprising plasma components from an individual (the “donor individual”, or “donor”) to an individual at least at risk of suffering or suffering from cognitive impairment and/or age-related dementia or a postoperative condition (the “recipient individual” or “recipient”). By a “blood product comprising plasma components,” it is meant any product derived from blood that comprises plasma (e.g. whole blood, blood plasma, or fractions thereof). The term “plasma” is used in its conventional sense to refer to the straw-colored/pale-yellow liquid component of blood composed of about 92% water, 7% proteins such as albumin, gamma globulin, anti-hemophilic factor, and other clotting factors, and 1% mineral salts, sugars, fats, hormones and vitamins. Non-limiting examples of plasma-comprising blood products suitable for use in the subject methods include whole blood treated with anti-coagulant (e.g., EDTA, citrate, oxalate, heparin, etc.), blood products produced by filtering whole blood to remove white blood cells (“leukoreduction”), blood products consisting of plasmapheretically-derived or apheretically-derived plasma, fresh-frozen plasma, blood products consisting essentially of purified plasma, and blood products consisting essentially of plasma fractions. In some instances, plasma product that is employed is a non-whole blood plasma product, by which is meant that the product is not whole blood, such that it lacks one or more components found in whole blood, such as erythrocytes, leukocytes, etc., at least to the extent that these components are present in whole blood. In some instances, the plasma product is substantially, if not completely, acellular, where in such instances the cellular content may be 5% by volume or less, such as 1% or less, including 0.5% or less, where in some instances acellular plasma fractions are those compositions that completely lack cells, i.e., they include no cells.

Collection of blood products comprising plasma components. Embodiments of the methods described herein include administration of blood products comprising plasma components which can be derived from donors, including human volunteers. The term, “human-derived” can refer to such products. Methods of collection of plasma comprising blood products from donors are well-known in the art. (See, e.g., AABB TECHNICAL MANUAL, (Mark A. Fung, et al., eds., 18th ed. 2014), herein incorporated by reference).

In one embodiment, donations are obtained by venipuncture. In another embodiment, the venipuncture is only a single venipuncture. In another embodiment, no saline volume replacement is employed. In an embodiment, the process of plasmapheresis is used to obtain the plasma comprising blood products. Plasmapheresis can comprise the removal of a weight-adjusted volume of plasma with the return of cellular components to the donor. In an embodiment, sodium citrate is used during plasmapheresis in order to prevent cell clotting. The volume of plasma collected from a donor is preferably between 690 to 880 mL after citrate administration, and preferably coordinates with the donor's weight.

### 3. Blood Plasma Fractions

During the Second World War, there arose a need for a stable plasma expander which could be employed in the battlefield when soldiers lost large amounts of blood. As a result, methods of preparing freeze-dried plasma were developed. However, use of freeze-dried plasma was difficult in combat situations since reconstitution required sterile water. As an alternative, Dr. E. J. Cohn suggested that albumin could be used, and prepared a ready-to-use stable solution that could be introduced immediately for treatment of shock. (See JOHAN VANDERSANDE, CURRENT APPROACHES TO THE PREPARATION OF PLASMA FRACTIONS in (BIOTECHNOLOGY OF BLOOD) 165 (Jack Goldstein ed., 1st ed. 1991)). Dr. Cohn's procedure of purifying plasma fractions utilized cold ethanol for its denaturing effect, and employs changes in pH and temperature to achieve separation.

An embodiment of the methods described herein includes the administration of plasma fractions to a subject. Fractionation is the process by which certain protein subsets are separated from plasma. Fractionation technology is known in the art and relies on steps developed by Cohn et al. during the 1940s. (E. Cohn, Preparation and properties of serum and plasma proteins. IV. A system for the separation into fractions of the protein and lipoprotein components of biological tissues and fluids. 68 J Am Chem Soc 459 (1946), herein incorporated by reference). Several steps are involved in this process, each step involving specific ethanol concentrations as well as pH, temperature, and osmolality shifts which result in selective protein precipitation. Precipitates are also separated via centrifugation or precipitation. The original “Cohn fractionation process” involved separation of proteins through precipitates into five fractions, designated fraction I, fraction II+III, fraction IV-1, fraction IV-4 and fraction V. Albumin was the originally identified endpoint (fraction V) product of this process. In accordance with embodiments of the invention, each fraction (or effluent from a prior separation step) contains or potentially contains therapeutically-useful protein fractions. (See Thierry Burnouf, Modern Plasma Fractionation, 21(2) Transfusion Medicine Reviews 101 (2007); Adil Denizli, Plasma fractionation: conventional and chromatographic methods for albumin purification, 4 J. Biol. & Chem. 315, (2011); and T. Brodniewicz-Proba, Human Plasma Fractionation and the Impact of New Technologies on the Use and Quality of Plasma-derived Products, 5 Blood Reviews 245 (1991), and U.S. Pat. Nos. 3,869,431, 5,110,907, 5,219,995, 7,531,513, and 8,772,461 which are herein incorporated by reference). Adjustment of the above experimental parameters can be made in order to obtain specific protein fractions.

More recently, fractionation has reached further complexity, and as such, comprises additional embodiments of the invention. This recent increase in complexity has occurred through: the introduction of chromatography resulting in isolation of new proteins from existing fractions like cryoprecipitate, cryo-poor plasma, and Cohn fractions; increasing IgG recovery by integrating chromatography and the ethanol fractionation process; and viral reduction/inactivation/removal. (Id.) In order to capture proteins at physiological pH and ionic strength, anion-exchange chromatography can be utilized. This preserves functional activity of proteins and/or protein fractions. Heparin and monoclonal antibodies are also used in affinity chromatography. Additionally, fractionation using gel filtration, fraction by salt, and fractionation by polyethylene glycol are used. (Hosseini M Iran J Biotech, 14(4): 213-20 (2016) herein incorporated by reference). One of ordinary skill in the art would recognize that the parameters and techniques described above may be adjusted to obtain specifically-desired plasma protein-containing fractions.

Blood plasma fractionation can also be ammonium sulfate-based. (See, e.g., Odunuga O O, Biochem Compounds, 1:3 (2013); Wingfield P T, Curr Protoc Protein Sci, Appx. 3 (2001), herein incorporated by reference). In addition to obtaining specific blood fractions, ammonium sulfate-based fractionation has been employed to reduce abundant proteins from plasma. (Saha S, et al., J. Proteomics Bioinform, 5(8) (2012), herein incorporated by reference).

In an embodiment of the invention, blood plasma is fractionated in an industrial setting. Frozen plasma is thawed at 1° C. to 4° C. Continuous refrigerated centrifugation is applied to the thawed plasma and cryoprecipitate isolated. Recovered cryoprecipitate is frozen at −30° C. or lower and stored. The cryoprecipitate-poor (“cryo-poor”) plasma is immediately processed for capture (via, for example, primary chromatography) of labile coagulation factors such as factor IX complex and its components as well as protease inhibitors such as antithrombin and C1 esterase inhibitor. Serial centrifugation and precipitate isolation can be applied in subsequent steps. Such techniques are known to one of ordinary skill in the art and are described, for example, in U.S. Pat. Nos. 4,624,780, 5,219,995, 5,288,853, and U.S. patent application nos. 20140343255 and 20150343025, which disclosures are incorporated by reference in their entirety herein.

In an embodiment of the invention, the plasma fraction may comprise a plasma fraction containing a substantial concentration of albumin. In another embodiment of the invention, the plasma fraction may comprise a plasma fraction containing a substantial concentration of IgG or intravenous immune globulin (IGIV) (e.g. Gamunex-C®). In another embodiment of the invention the plasma fraction may comprise an IGIV plasma fraction, such as Gamunex-C® which has been substantially depleted of immune globulin (IgG) by methods well-known by one of ordinary skill in the art, such as for example, Protein A-mediated depletion. (See Keshishian, H., et al., Multiplexed, Quantitative Workflow for Sensitive Biomarker Discovery in Plasma Yields Novel Candidates for Early Myocardial Injury, Molecular & Cellular Proteomics, 14 at 2375-93 (2015)). In an additional embodiment, the blood plasma fraction may be one in which substantially all the clotting factors are removed in order to retain the efficacy of the fraction with reduced risk of thromboses. For example, the plasma fraction may be a plasma fraction as described in U.S. Patent No. 62/376,529 filed on Aug. 18, 2016; the disclosure of which is incorporated by reference in its entirety herein.

### 4. Albumin Products

To those having ordinary skill in the art, there are two general categories of Albumin Plasma Products (“APP”): plasma protein fraction (PPF) and human albumin solution (HAS). PPF is derived from a process with a higher yield than HAS, but has a lower minimum albumin purity than HAS (>83% for PPF and >95% for HAS). (Production of human albumin solution: a continually developing colloid, P. Matejtschuk et al., British J. of Anaesthesia 85(6): 887-95, at 888 (2000)). In some instances, PPF has albumin purity of between 83% and 95% or alternatively 83% and 96%. The albumin purity can be determined by electrophoresis or other quantifying assays such as, for example, by mass spectrometry. Additionally, some have noted that PPF has a disadvantage because of the presence of protein “contaminants” such as PKA. Id. As a consequence, PPF preparations have lost popularity as Albumin Plasma Products, and have even been delisted from certain countries' Pharmacopoeias. Id. Contrary to these concerns, the invention makes beneficial use of these “contaminants.” Besides α, β, and γ globulins, as well as the aforementioned PKA, the methods of the invention utilize additional proteins or other factors within the “contaminants” that promote processes such as neurogenesis, neuronal cell survival, and improved cognition or motor function and decreased neuroinflammation.

Those of skill in the art will recognize that there are, or have been, several commercial sources of PPF (the “Commercial PPF Preparations.”) These include Plasma-Plex™ PPF (Armour Pharmaceutical Co., Tarrytown, N.Y.), Plasmanate™ PPF (Grifols, Clayton, N.C.), Plasmatein™ (Alpha Therapeutics, Los Angeles, Calif.), and Protenate™ PPF (Baxter Labs, Inc. Deerfield, Ill.).

Those of skill in the art will also recognize that there are, or have been, several commercial sources of HAS (the “Commercial HAS Preparations.”) These include Albuminar™ (CSL Behring), AlbuRx™ (CSL Behring), Albutein™ (Grifols, Clayton, N.C.), Buminate™ (Baxatla, Inc., Bannockburn, Ill.), Flexbumin™ (Baxalta, Inc., Bannockburn, Ill.), and Plasbumin™ (Grifols, Clayton, N.C.).

A. Plasma Protein Fraction (Human) (PPF)

According to the United States Food and Drug Administration (“FDA”), “Plasma Protein Fraction (Human),” or PPF, is the proper name of the product defined as “a sterile solution of protein composed of albumin and globulin, derived from human plasma.” (Code of Federal Regulations “CFR” 21 CFR 640.90 which is herein incorporated by reference). PPF's source material is plasma recovered from Whole Blood prepared as prescribed in 21 CFR 640.1-640.5 (incorporated by reference herein), or Source Plasma prepared as prescribed in 21 CFR 640.60-640.76 (incorporated by reference herein).

PPF is tested to determine it meets the following standards as per 21 CFR 640.92 (incorporated by reference herein):
(a) The final product shall be a 5.0+/−0.30 percent solution of protein; and
(b) The total protein in the final product shall consist of at least 83 percent albumin, and no more than 17 percent globulins. No more than 1 percent of the total protein shall be gamma globulin. The protein composition is determined by a method that has been approved for each manufacturer by the Director, Center for Biologics Evaluation and Research, Food and Drug Administration.

As used herein, “Plasma Protein Fraction” or “PPF” refers to a sterile solution of protein composed of albumin and globulin, derived from human plasma, with an albumin content of at least 83% with no more than 17% globulins (including α1, α2, β, and γ globulins) and other plasma proteins, and no more than 1% gamma globulin as determined by electrophoresis. (Hink, J. H., Jr., et al., Preparation and Properties of a Heat-Treated Human Plasma Protein Fraction, VOX SANGUINIS 2(174) (1957)). PPF can also refer to a solid form, which when suspended in solvent, has similar composition. The total globulin fraction can be determined through subtracting the albumin from the total protein. (Busher, J., Serum Albumin and Globulin, CLINICAL METHODS: THE HISTORY, PHYSICAL, AND LABORATORY EXAMINATIONS, Chapter 10, Walker H K, Hall W D, Hurst J D, eds. (1990)).

B. Albumin (Human) (HAS)

According to the FDA, “Albumin (Human)” (also referred to herein as “HAS”) is the proper name of the product defined as “sterile solution of the albumin derived from human plasma.” (Code of Federal Regulations “CFR” 21 CFR 640.80 which is herein incorporated by reference.) The source material for Albumin (Human) is plasma recovered from Whole Blood prepared as prescribed in 21 CFR 640.1-640.5 (incorporated by reference herein), or Source Plasma prepared as prescribed in 21 CFR 640.60-640.76 (incorporated by reference herein). Other requirements for Albumin (Human) are listed in 21 CFR 640.80-640.84 (incorporated by reference herein).

Albumin (Human) is tested to determine if it meets the following standards as per 21 CFR 640.82:

(a) Protein concentration. Final product shall conform to one of the following concentrations: 4.0+/−0.25 percent; 5.0+/−0.30 percent; 20.0+/−1.2 percent; and 25.0+/−1.5 percent solution of protein.

(b) Protein composition. At least 96 percent of the total protein in the final product shall be albumin, as determined by a method that has been approved for each manufacturer by the Director, Center for Biologics Evaluation and Research, Food and Drug Administration.

As used herein, “Albumin (Human)” or “HAS” refers to a to a sterile solution of protein composed of albumin and globulin, derived from human plasma, with an albumin content of at least 95%, with no more than 5% globulins (including α1, α2, β, and γ globulins) and other plasma proteins. HAS can also refer to a solid form, which when suspended in solvent, has similar composition. The total globulin fraction can be determined through subtracting the albumin from the total protein.

As can be recognized by one having ordinary skill in the art, PPF and HAS fractions can also be freeze-dried or in other solid form. Such preparations, with appropriate additives, can be used to make tablets, powders, granules, or capsules, for example. The solid form can be formulated into preparations for injection by dissolving, suspending or emulsifying them in an aqueous or non-aqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

### 5. Clotting Factor-Reduced Fractions

Another embodiment of the invention uses a blood plasma fraction from which substantially all of the clotting factors are removed in order to retain the efficacy of the fraction with reduced risk of thromboses. Another embodiment of the invention uses a blood plasma fraction from which substantially all of the clotting factors are removed in order to retain the efficacy of the fraction with reduced risk of thromboses. Conveniently, the blood product can be derived from a young donor or pool of young donors, and can be rendered devoid of IgM in order to provide a young blood product that is ABO compatible. Currently, plasma that is transfused is matched for ABO blood type, as the presence of naturally occurring antibodies to the A and B antigens can result in transfusion reactions. IgM appears to be responsible for transfusion reactions when patients are given plasma that is not ABO matched. Removal of IgM from blood products or fractions helps eliminate transfusion reactions in subjects who are administered the blood products and blood plasma fractions of the invention.

Accordingly, in one embodiment, the invention is directed to a method of treating or preventing an aging-related condition such as cognitive impairment or neurodegeneration in a subject. The method comprises: administering to the subject a blood product or blood fraction derived from whole-blood from an individual or pool of individuals, wherein the blood product or blood fraction is substantially devoid of (a) at least one clotting factor and/or (b) IgM. In some embodiments, the individual(s) from whom the blood product or blood fraction is derived are young individuals. In some embodiments, the blood product is substantially devoid of at least one clotting factor and IgM. In certain embodiments, the blood product is substantially devoid of fibrinogen (Factor I). In additional embodiments, the blood product substantially lacks erythrocytes and/or leukocytes. In further embodiments, the blood product is substantially acellular. In other embodiments, the blood product is derived from plasma. Such embodiments of the invention are further supported by U.S. Patent Application No. 62/376,529 filed on Aug. 18, 2016, which is incorporated by reference in its entirety herein.

### 6. Protein-Enriched Plasma Protein Products

Additional embodiments of the invention use plasma fractions with reduced albumin concentration compared to PPF, but with increased amounts of globulins and other plasma proteins (what have been referred to by some as “contaminants”). The embodiments, as with PPF, HAS, Effluent I, and Effluent II/III are all effectively devoid of clotting factors. Such plasma fractions are hereinafter referred to as “protein-enriched plasma protein products.” For example, an embodiment of the invention may use a protein-enriched plasma protein product comprised of 82% albumin and 18% α, β, and γ globulins and other plasma proteins. Another embodiment of the invention may use a protein-enriched plasma protein product comprised of 81% albumin and 19% of α, β, and γ globulins and/or other plasma proteins. Another embodiment of the invention may use a protein-enriched plasma protein product comprised of 80% albumin and 20% of α, β, and γ globulins and/or other plasma proteins. Additional embodiments of the invention may use protein-enriched plasma protein products comprised of 70-79% albumin and a corresponding 21-30% of α, β, and γ globulins and other plasma proteins. Additional embodiments of the invention may use protein-enriched plasma protein products comprised of 60-69% albumin and a corresponding 31-40% of α, β, and γ globulins and other plasma proteins. Additional embodiments of the invention may use protein-enriched plasma protein products comprised of 50-59% albumin and a corresponding 41-50% of α, β, and γ globulins and other plasma proteins. Additional embodiments of the invention may use protein-enriched plasma protein products comprised of 40-49% albumin and a corresponding 51-60% of α, β, and γ globulins and other plasma proteins. Additional embodiments of the invention may use protein-enriched plasma protein products comprised of 30-39% albumin and a corresponding 61-70% of α, β, and γ globulins and other plasma proteins. Additional embodiments of the invention may use protein-enriched plasma protein products comprised of 20-29% albumin and a corresponding 71-80% of α, β, and γ globulins and other plasma proteins. Additional embodiments of the invention may use protein-enriched plasma protein products comprised of 10-19% albumin and a corresponding 81-90% of α, β, and γ globulins and other plasma proteins. Additional embodiments of the invention may use protein-enriched plasma protein products comprised of 1-9% albumin and a corresponding 91-99% of α, β, and γ globulins and other plasma proteins. A further embodiment of the invention may use protein-enriched plasma protein products comprised of 0-1% albumin and 99-100% of α, β, and γ globulins and other plasma proteins

Embodiments of the invention described above may also have total gamma globulin concentrations of 0-5%.

The specific concentrations of proteins in a plasma fraction may be determined using techniques well-known to a person having ordinary skill in the relevant art. By way of example, and not limitation, such techniques include electrophoresis, mass spectrometry, ELISA analysis, and Western blot analysis.

### 7. Preparation of Blood Plasma Fractions

Methods of preparing PPF and other plasma fractions are well-known to those having ordinary skill in the art. An embodiment of the invention allows for blood used in the preparation of human plasma protein fraction to be collected in flasks with citrate or anticoagulant citrate dextrose solution (or other anticoagulant) for inhibition of coagulation, with further separation of Fractions I, II+III, IV, and PPF as per the method disclosed in Hink et al. (See Hink, J. H., Jr., et al., Preparation and Properties of a Heat-Treated Human Plasma Protein Fraction, VOX SANGUINIS 2(174) (1957), herein incorporated by reference.) According to this method, the mixture can be collected to 2-8° C. The plasma can then subsequently be separated by centrifugation at 7° C., removed, and stored at −20° C. The plasma can then be thawed at 37° C. and fractionated, preferably within eight hours after removal from −20° C. storage.

Plasma can be separated from Fraction I using 8% ethanol at pH 7.2 and a temperature at −2 to −2.5° C. with protein concentration of 5.1 to 5.6 percent. Cold 53.3 percent ethanol (176 mL/L of plasma) with acetate buffer (200 mL 4M sodium acetate, 230 mL glacial acetic acid quantum satis to 1 L with H₂O) can be added using jets at a rate, for example, of 450 mL/minute during the lowering the plasma temperature to −2° C. Fraction I can be separated and removed from the effluent (Effluent I) through ultracentrifugation. Fibrinogen can be obtained from Fraction I as per methods well-known to those having ordinary skill in the art.

Fraction II+III can be separated from Effluent I through adjustment of the effluent to 21 percent ethanol at pH 6.8, temperature at −6° C., with protein concentration of 4.3 percent. Cold 95 percent ethanol (176 mL/L of Effluent I) with 10 M acetic acid used for pH adjustment can be added using jets at a rate, for example, of 500 mL/minute during the lowering of the temperature of Effluent I to −6° C. The resulting precipitate (Fraction II+III) can be removed by centrifugation at −6° C. Gamma globulin can be obtained from Fraction II+III using methods well-known to those having ordinary skill in the art.

Fraction IV-1 can be separated from Effluent II+III (“Effluent II/III”) through adjustment of the effluent to 19 percent ethanol at pH 5.2, temperature at −6° C., and protein concentration of 3 percent. H₂0 and 10 M acetic acid used for pH adjustment can be added using jets while maintaining Effluent II/III at −6° C. for 6 hours. Precipitated Fraction VI-1 can be settled at −6° C. for 6 hours and subsequently separated from the effluent by centrifugation at the same temperature. Stable plasma protein fraction can be recovered from Effluent IV-1 through adjustment of the ethanol concentration to 30 percent at pH 4.65, temperature −7° C. and protein concentration of 2.5 percent. This can be accomplished by adjusting the pH of Effluent IV-1 with cold acid-alcohol (two parts 2 M acetic acid and one part 95 percent ethanol). While maintaining a temperature of −7° C., to every liter of adjusted Effluent IV-1 170 mL cold ethanol (95%) is added. Proteins that precipitate can be allowed to settle for 36 hours and subsequently removed by centrifugation at −7° C.

The recovered proteins (stable plasma protein fraction) can be dried (e.g. by freeze drying) to remove alcohol and H₂0. The resulting dried powder can be dissolved in sterile distilled water, for example using 15 liters of water/kg of powder, with the solution adjusted to pH 7.0 with 1 M NaOH. A final concentration of 5 percent protein can be achieved by adding sterile distilled water containing sodium acetyl tryptophanate, sodium caprylate, and NaCl, adjusting to final concentrations of 0.004 M acetyl tryptophanate, 0.004 M caprylate, and 0.112 M sodium. Finally, the solution can be filtered at 10° C. to obtain a clear solution and subsequently heat-treated for inactivation of pathogens at 60° C. for at least 10 hours.

One having ordinary skill in the art would recognize that each of the different fractions and effluents described above could be used with the methods of the invention to treat a disease or a condition associated with postoperative recovery. For example, and not by way of limitation, Effluents I or Effluent II/III may be utilized to treat such diseases as cognitive and neurodegenerative disorders or conditions associated with postoperative recovery or to accelerate postoperative recovery and are embodiments of the invention.

The preceding methods of preparing blood plasma fractions and plasma protein fraction (PPF) are only exemplary and involves merely embodiments of the invention. One having ordinary skill in the art would recognize that these methods can vary. For example, pH, temperature, and ethanol concentration, among other things can be adjusted to produce different variations of plasma fractions and plasma protein fraction in the different embodiments and methods of the invention. In another example, additional embodiments of the invention contemplate the use of nanofiltration for the removal/inactivation of pathogens from plasma fractions and plasma protein fraction.

An additional embodiment of the invention contemplates methods and composition using and/or comprising additional blood plasma fractions. For example, the invention, among other things, demonstrates that specific concentrations of albumin are not critical for improving cognitive activity or treating conditions associated with postoperative recovery or for accelerating postoperative recovery. Hence, fractions with reduced albumin concentration, such as those fractions having below 83% albumin, are contemplated by the invention.

### 8. Treatment

Aspects of the methods of the inventions described herein include treatment of a subject with a plasma comprising blood product, such as a blood plasma fraction, e.g., as described above. An embodiment includes treatment of a human subject with a plasma comprising blood product. One of skill in the art would recognize that methods of treatment of subjects with plasma comprising blood products are recognized in the art. By way of example, and not limitation, one embodiment of the methods of the inventions described herein is comprised of administering fresh frozen plasma to a subject for treatment and/or prevention of cognitive impairment and/or age-related dementia or of conditions associated with postoperative recovery. In one embodiment, the plasma comprising blood product is administered immediately, e.g., within about 12-48 hours of collection from a donor, to the individual suffering or at risk from a cognitive impairment and/or age-related dementia or from a condition associated with postoperative recovery. In such instances, the product may be stored under refrigeration, e.g., 0-10° C. In another embodiment, fresh frozen plasma is one that has been stored frozen (cryopreserved) at −18° C. or colder. Prior to administration, the fresh frozen plasma is thawed and once thawed, administered to a subject 60-75 minutes after the thawing process has begun. Each subject preferably receives a single unit of fresh frozen plasma (200-250 mL), the fresh frozen plasma preferably derived from donors of a pre-determined age range. In one embodiment of the invention, the fresh frozen plasma is donated by (derived from) young individuals. In another embodiment of the invention, the fresh frozen plasma is donated by (derived from) donors of the same gender. In another embodiment of the invention, the fresh frozen plasma is donated by (derived from) donors of the age range between 18-22 years old.

In an embodiment of the invention the compositions of the invention the compositions (e.g. plasma comprising blood product, such as a blood plasma fraction) are administered intravenously. The compositions of the invention may also be delivered intraperitoneally. In another embodiment of the invention, the compositions of the invention may be delivered per os, subcutaneously, or topically. Topical formulations for treating wounds and promoting would healing as known in the art as gels, creams, ointments, gauze, patches and the like, and the compositions of the invention may be formulated as such. (See, e.g., Kahn A W, et al., Pharmacogn Mag, 9(Suppl 1):S6-S10 (2013); U.S. Pat. Nos. 5,641,483; 4,885,163; 8,313,764, which are incorporated herein in their entirety).

In an embodiment of the invention, the plasma comprising blood products are screened after donation by blood type. In another embodiment of the invention, the plasma comprising blood products are screened for infectious disease agents such as HIV I & II, HBV, HCV, HTLV I & II, anti-HBc per the requirements of 21 CFR 640.33 and recommendations contained in FDA guidance documents.

In yet another embodiment of the invention, the subject is treated with a Plasma Fraction. In an embodiment of the invention, the plasma fraction is a PPF or a HAS. In a further embodiment of the invention, the plasma fraction is one of the Commercial PPF Preparations of the Commercial HAS Preparations. In another embodiment of the invention the plasma fraction is a PPF or HAS derived from a pool of individuals of a specific age range, such as young individuals, or is a modified PPF or HAS fraction which has been subjected to additional fractionation or processing (e.g. PPF or HAS with one or more specific proteins partially or substantially removed). In another embodiment of the invention, the plasma fraction is an IGIV plasma fraction which has been substantially depleted of immune globulin (IgG). A blood fraction which is “substantially depleted” or which has specific proteins “substantially removed,” such as IgG, refers to a blood fraction containing less than about 50% of the amount that occurs in the reference product or whole blood plasma, such as less than 45%, 40%, 35%, 30%, 25%, 20%, 15%, 5%, 4%, 3%, 2%, 1%, 0.5%, 0.25%, 0.1%, undetectable levels, or any integer between these values, as measured using standard assays well known in the art.

Aspects of the methods of the inventions described herein include treatment of a subject with a plasma comprising blood product, such as a blood plasma or Plasma Fraction, e.g., as described above. An embodiment includes treatment of a human subject with a plasma comprising blood product. One of skill in the art would recognize that methods of treatment of subjects with plasma comprising blood products are recognized in the art. By way of example, and not limitation, one embodiment of the methods of the inventions described herein is comprised of administering fresh frozen plasma to a subject for treatment of conditions associated with postoperative recovery. In one embodiment, the plasma comprising blood product is administered immediately, e.g., within about 12-48 hours of collection from a donor, to the individual suffering from an unwanted condition associated with postoperative recovery.

In such instances, the product may be stored under refrigeration, e.g., 0-10° C. In another embodiment, fresh frozen plasma is one that has been stored frozen (cryopreserved) at −18° C. or colder. Prior to administration, the fresh frozen plasma is thawed and once thawed, administered to a subject 60-75 minutes after the thawing process has begun. Each subject preferably receives a single unit of fresh frozen plasma (200-250 mL), the fresh frozen plasma preferably derived from donors of a pre-determined age range. In one embodiment of the invention, the fresh frozen plasma is donated by (derived from) young individuals. In another embodiment of the invention, the fresh frozen plasma is donated by (derived from) donors of the same gender. In another embodiment of the invention, the fresh frozen plasma is donated by (derived from) donors of the age range between 18-22 years old. In one embodiment, subjects are treated twice per week with 3-4 days between infusions. In an embodiment of the invention, treatment persists until a specific endpoint is reached.

In an embodiment of the invention, the plasma comprising blood products are screened after donation by blood type. In another embodiment of the invention, the plasma comprising blood products are screened for infectious disease agents such as HIV I & II, HBV, HCV, HTLV I & II, anti-HBc per the requirements of 21 CFR 640.33 and recommendations contained in FDA guidance documents.

In yet another embodiment of the invention, the subject is treated with a “Plasma Fraction.” In an embodiment of the invention, the Plasma Fraction is PPF or HAS. In a further embodiment of the invention, the Plasma Fraction is one of the Commercial PPF Preparations of the Commercial HAS Preparations. In another embodiment of the invention the Plasma Fraction is a PPF or HAS derived from a pool of individuals of a specific age range, such as young individuals, or is a modified PPF or HAS fraction which has been subjected to additional fractionation or processing (e.g. PPF or HAS with one or more specific proteins partially or substantially removed). In another embodiment of the invention, the Plasma Fraction is an IGIV plasma fraction which has been substantially depleted of immune globulin (IgG). A blood fraction which is “substantially depleted” or which has specific proteins “substantially removed,” such as IgG, refers to a blood fraction containing less than about 50% of the amount that occurs in the reference product or whole blood plasma, such as less than 45%, 40%, 35%, 30%, 25%, 20%, 15%, 5%, 4%, 3%, 2%, 1%, 0.5%, 0.25%, 0.1%, undetectable levels, or any integer between these values, as measured using standard assays well known in the art.

### 9. Monitoring

Another aspect of the present invention relates to methods of monitoring the effect of a medication on a subject for treating cognitive impairment and/or age-related dementia, the method comprising comparing cognitive function before and after treatment. Those having ordinary skill in the art recognize that there are well-known methods of evaluating cognitive function. For example, and not by way of limitation, the method may comprise evaluation of cognitive function based on medical history, family history, physical and neurological examinations by clinicians who specialize dementia and cognitive function, laboratory tests, and neuropsychological assessment. Additional embodiments which are contemplated by the invention include: the assessment of consciousness, such as using the Glasgow Coma Scale (EMV); mental status examination, including the abbreviated mental test score (AMTS) or mini-mental state examination (MMSE) (Folstein et al., J. Psychiatr. Res 1975; 12:1289-198); global assessment of higher functions; estimation of intracranial pressure such as by fundoscopy.

In one embodiment, examinations of peripheral nervous system may be used to evaluate cognitive function, including any one of the followings: sense of smell, visual fields and acuity, eye movements and pupils (sympathetic and parasympathetic), sensory function of face, strength of facial and shoulder girdle muscles, hearing, taste, pharyngeal movement and reflex, tongue movements, which can be tested individually (e.g. the visual acuity can be tested by a Snellen chart; a reflex hammer used testing reflexes including masseter, biceps and triceps tendon, knee tendon, ankle jerk and plantar (i.e. Babinski sign); Muscle strength often on the MRC scale 1 to 5; Muscle tone and signs of rigidity.

### 10. Administration

In practicing methods of the invention, a blood plasma fraction is administered to the subject. In an embodiment, the blood plasma fraction is administered by intravenous infusion. The rate of infusion may vary, but in one embodiment of the invention, the infusion rate is 5-8 mL/minute. Those having ordinary skill in the art will recognize that the infusion rate can depend upon the subject's condition and response to administration.

In those embodiments where an effective amount of an active agent is administered to the adult mammal, the amount or dosage is effective when administered for a suitable period of time, such as one week or longer, including two weeks or longer, such as 3 weeks or longer, one month or longer, 2 months or longer, 3 months or longer, 4 months or longer, 5 months or longer, 6 months or longer, 1 year or longer etc., so as to evidence a reduction in the condition, e.g., cognitive impairment, or delay of cognitive impairment and/or cognitive improvement in the adult mammal. For example, an effective dose is the dose that, when administered for a suitable period of time, will slow e.g., by about 20% or more, e.g., by 30% or more, by 40% or more, or by 50% or more, in some instances by 60% or more, by 70% or more, by 80% or more, or by 90% or more. For example, will halt, cognitive decline in a patient suffering from natural aging or an aging-associated disorder or a condition with postoperative recovery by administering to the subject an effective amount of blood plasma. In some instances, an effective amount or dose of blood product will not only slow or halt the progression of the disease condition but will also induce the reversal of the condition, i.e., will cause an improvement in cognitive ability. For example, in some instances, an effective amount is the amount that when administered for a suitable period of time, usually at least about one week, and maybe about two weeks, or more, up to a person of about 3 weeks, 4 weeks, 8 weeks, or longer will improve the cognitive abilities of an individual suffering from an aging-associated cognitive impairment by, for example, 1.5-fold, 2-fold, 3-fold, 4-fold, 5-fold, in some instances 6-fold, 7-fold, 8-fold, 9-fold, or 10-fold or more relative to cognition prior to administration of the blood product or fraction. In some instances, an effective amount or dose of active agent will not only slow or halt the progression of the disease condition but will also induce the reversal of the condition, i.e., will cause an improvement in cognitive function. For example, in some instances, an effective amount is the amount that when administered for a suitable period of time, will improve the symptoms an individual suffering from cognitive decline or impairment, for example 1.5-fold, 2-fold, 3-fold, 4-fold, 5-fold, in some instances 6-fold, 7-fold, 8-fold, 9-fold, or 10-fold or more relative to untreated individuals prior to administration of the agent.

In other embodiments, the blood plasma fraction or Plasma Fraction is administered in accordance with one or more dosing regimens described in U.S. Patent Application No. 62/490,519, which is herein incorporated by reference in its entirety. As such, an embodiment of the invention includes treating a subject diagnosed with a cognitive impairment or suffering from a condition associated with postoperative recovery by administering to the subject an effective amount of blood plasma or Plasma Fraction wherein the blood plasma or Plasma Fraction is administered in a manner resulting in improved cognitive function or neurogenesis or improved wound healing, the presence of markers, decreased pain, or decreased inflammation after the mean or median half-life of the blood plasma proteins or Plasma Fraction proteins been reached, relative to the most recent administered dose (referred to as “Pulsed Dosing” or “Pulse Dosed” herein) (See U.S. Pat. No. 10,357,513 and U.S. patent application Ser. No. 15/961,618 and 62/701,411, which are herein incorporated by reference in their entirety).

Another embodiment of the invention includes administering the effective amount of blood plasma or Plasma Fraction and subsequently monitoring the subject for improved function, wound healing, the presence of markers, decreased pain, or decreased inflammation.

Another embodiment of the invention includes administering the blood plasma or Plasma Fraction via a dosing regimen of at least two consecutive days and monitoring the subject for improved cognitive function or HSC marker levels at least 3 days after the date of last administration. A further embodiment of the invention includes administering the blood plasma or Plasma Fraction via a dosing regimen of at least 3, 4, 5, 6, 7, 8, 9, 10, 11, 12, 13, or 14 consecutive days and monitoring the subject for improved cognitive function, wound healing, the presence of markers, decreased pain, or decreased inflammation at least 3 days after the date of last administration. Yet another embodiment of the invention includes administering the blood plasma or Plasma Fraction via a dosing regimen of at least 2 consecutive days and after the date of last administration, monitoring for cognitive improvement, functional improvement, wound healing, the presence of markers, decreased pain, or decreased inflammation beyond when the average half-life of the proteins in the blood plasma or Plasma Fraction has been reached. Another embodiment of the invention includes administering the blood plasma or Plasma Fraction via a dosing regimen of 2 to 14 non-consecutive days wherein each gap between doses may be between 0-3 days each.

In some instances, Pulsed Dosing in accordance with the invention includes administration of a first set of doses, e.g., as described above, followed by a period of no dosing, e.g., a “dosing-free period”, which in turn is followed by administration of another dose or set of doses. The duration of this “dosing-free” period, may vary, but in some embodiments, is 7 days or longer, such as 10 days or longer, including 14 days or longer, wherein some instances the dosing-free period ranges from 15 to 365 days, such as 30 to 90 days and including 30 to 60 days. As such, embodiments of the methods include non-chronic (i.e., non-continuous) dosing, e.g., non-chronic administration of a blood plasma product. In some embodiments, the pattern of Pulsed Dosing followed by a dosing-free period is repeated for a number of times, as desired, where in some instances this pattern is continued for 1 year or longer, such as 2 years or longer, up to and including the life of the subject. Another embodiment of the invention includes administering the blood plasma or Plasma Fraction via a dosing regimen of 5 consecutive days, with a dosing-free period of 2-3 days, followed by administration for 2-14 consecutive days.

Biochemically, by an “effective amount” or “effective dose” of active agent is meant an amount of active agent that will inhibit, antagonize, decrease, reduce, or suppress by about 20% or more, e.g., by 30% or more, by 40% or more, or by 50% or more, in some instances by 60% or more, by 70% or more, by 80% or more, or by 90% or more, in some cases by about 100%, i.e., to negligible amounts, and in some instances, reverse the progression of the cognitive impairment or age-associated dementia or reverse unwanted conditions with postoperative recovery.

### 11. Plasma Protein Fraction

In practicing methods of the invention, a plasma fraction is administered to the subject. In an embodiment, the Plasma Fraction is plasma protein fraction (PPF). In additional embodiments, the PPF is selected from the Commercial PPF Preparations.

In another embodiment, the PPF is comprised of 88% normal human albumin, 12% alpha and beta globulins and not more than 1% gamma globulin as determined by electrophoresis. Further embodiments used in practicing methods of the invention include, for example, using a 5% solution of PPF buffered with sodium carbonate and stabilized with 0.004 M sodium caprylate and 0.004 M acetyltryptophan. Additional formulations, including those modifying the percentage of PPF (e.g. about 1% to about 10%, about 10% to about 20%, about 20% to 25%, about 25% to 30%) in solution as well as the concentrations of solvent and stabilizers may be utilized in practicing methods of the invention.

### 12. Plasma Fractions of Specific Donor Age

An embodiment of invention includes administering a blood plasma fraction or a Plasma Fraction derived from the plasma of individuals of certain age ranges. Additional embodiments of the invention include administering a plasma protein fraction derived from the plasma of individuals of certain age ranges. An embodiment includes administering a PPF or a HAS which has been derived from the plasma of young individuals. In another embodiment of the invention the young individuals are of a single specific age or a specific age range. In yet another embodiment, the average age of the donors is less than that of the subject or less than the average age of the subjects being treated.

Certain embodiments of the invention include pooling blood or blood plasma from individuals of specific age ranges and fractionating the blood plasma as described above to attain a plasma protein fraction product such as PPF or HAS. In an alternate embodiment of the invention, the plasma protein fraction or specific plasma protein fraction is attained from specific individuals fitting a specified age range. In another embodiment of the invention, the blood plasma fraction, Plasma Fraction, or specific plasma protein fraction product is attained from a pool of young individuals, of which “young” may be determined by chronologic or biologic age as described above, and the age(s) of the individuals may be a specific age or age range.

### 13. Indications

The subject methods and plasma-comprising blood products and fractions find use in treating, including preventing, aging-associated conditions, such as impairments in the cognitive ability of individuals, e.g., cognitive disorders, including (but not limited to) age-associated dementia, immunological conditions, cancer, and physical and functional decline. Individuals suffering from or at risk of developing an aging-associated cognitive impairment that will benefit from treatment with the subject plasma-comprising blood product, e.g., by the methods disclosed herein, include individuals that are about 50 years old or older, e.g., 60 years old or older, 70 years old or older, 80 years old or older, 90 years old or older, and 100 years old or older, i.e., between the age of about 50 and 100, e.g., 50, 55, 60, 65, 70, 75, 80, 85, 90, 95 or about 100 years old, and are suffering from cognitive impairment associated with natural aging process, e.g., mild cognitive impairment (M.C.I.); and individuals that are about 50 years old or older, e.g., 60 years old or older, 70 years old or older, 80 years old or older, 90 years old or older, and usually no older than 100 years old, i.e., between the ages of about 50 and 90, e.g., 50, 55, 60, 65, 70, 75, 80, 85, 90, 95 or about 100 years old, that have not yet begun to show symptoms of cognitive impairment. Examples of cognitive impairments that are due to natural aging include the following:

A. Mild cognitive impairment (M.C.I.) is a modest disruption of cognition that manifests as problems with memory or other mental functions such as planning, following instructions, or making decisions that have worsened over time while overall mental function and daily activities are not impaired. Thus, although significant neuronal death does not typically occur, neurons in the aging brain are vulnerable to sub-lethal age-related alterations in structure, synaptic integrity, and molecular processing at the synapse, all of which impair cognitive function.

Individuals suffering from or at risk of developing an aging-associated cognitive impairment that will benefit from treatment with the subject plasma-comprising blood product or fraction, e.g., by the methods disclosed herein, also include individuals of any age that are suffering from a cognitive impairment due to an aging-associated disorder; and individuals of any age that have been diagnosed with an aging-associated disorder that is typically accompanied by cognitive impairment, where the individual has not yet begun to present with symptoms of cognitive impairment. Examples of such aging-associated disorders include the following:

B. Alzheimer's disease. Alzheimer's disease is a progressive, inexorable loss of cognitive function associated with an excessive number of senile plaques in the cerebral cortex and subcortical gray matter, which also contains b-amyloid and neurofibrillary tangles consisting of tau protein. The common form affects persons>60 yr old, and its incidence increases as age advances. It accounts for more than 65% of the dementias in the elderly.

The cause of Alzheimer's disease is not known. The disease runs in families in about 15 to 20% of cases. The remaining, so-called sporadic cases have some genetic determinants. The disease has an autosomal dominant genetic pattern in most early-onset and some late-onset cases but a variable late-life penetrance. Environmental factors are the focus of active investigation.

In the course of the disease, synapses, and ultimately neurons are lost within the cerebral cortex, hippocampus, and subcortical structures (including selective cell loss in the nucleus basalis of Meynert), locus coeruleus, and nucleus raphae dorsalis. Cerebral glucose use and perfusion is reduced in some areas of the brain (parietal lobe and temporal cortices in early-stage disease, prefrontal cortex in late-stage disease). Neuritic or senile plaques (composed of neurites, astrocytes, and glial cells around an amyloid core) and neurofibrillary tangles (composed of paired helical filaments) play a role in the pathogenesis of Alzheimer's disease. Senile plaques and neurofibrillary tangles occur with normal aging, but they are much more prevalent in persons with Alzheimer's disease.

C. Parkinson's Disease. Parkinson's Disease (PD) is an idiopathic, slowly progressive, degenerative CNS disorder characterized by slow and decreased movement, muscular rigidity, resting tremor, and postural instability. Originally considered primarily a motor disorder, PD is now recognized to also affect cognition, behavior, sleep, autonomic function, and sensory function. The most common cognitive impairments include an impairment in attention and concentration, working memory, executive function, producing language, and visuospatial function.

In primary Parkinson's disease, the pigmented neurons of the substantia nigra, locus coeruleus, and other brain stem dopaminergic cell groups are lost. The cause is not known. The loss of substantia nigra neurons, which project to the caudate nucleus and putamen, results in depletion of the neurotransmitter dopamine in these areas. Onset is generally after age 40, with increasing incidence in older age groups.

Secondary parkinsonism results from loss of or interference with the action of dopamine in the basal ganglia due to other idiopathic degenerative diseases, drugs, or exogenous toxins. The most common cause of secondary parkinsonism is ingestion of antipsychotic drugs or reserpine, which produce parkinsonism by blocking dopamine receptors. Less common causes include carbon monoxide or manganese poisoning, hydrocephalus, structural lesions (tumors, infarcts affecting the midbrain or basal ganglia), subdural hematoma, and degenerative disorders, including striatonigral degeneration.

D. Frontotemporal dementia. Frontotemporal dementia (FTD) is a condition resulting from the progressive deterioration of the frontal lobe of the brain. Over time, the degeneration may advance to the temporal lobe. Second only to Alzheimer's disease (AD) in prevalence, FTD accounts for 20% of pre-senile dementia cases. Symptoms are classified into three groups based on the functions of the frontal and temporal lobes affected:

Behavioral variant FTD (bvFTD), with symptoms include lethargy and aspontaneity on the one hand, and disinhibition on the other; progressive nonfluent aphasia (PNFA), in which a breakdown in speech fluency due to articulation difficulty, phonological and/or syntactic errors is observed but word comprehension is preserved; and semantic dementia (SD), in which patients remain fluent with normal phonology and syntax but have increasing difficulty with naming and word comprehension. Other cognitive symptoms common to all FTD patients include an impairment in executive function and ability to focus. Other cognitive abilities, including perception, spatial skills, memory and praxis typically remain intact. FTD can be diagnosed by observation of reveal frontal lobe and/or anterior temporal lobe atrophy in structural MRI scans.

A number of forms of FTD exist, any of which may be treated or prevented using the subject methods and compositions. For example, one form of frontotemporal dementia is Semantic Dementia (SD). SD is characterized by a loss of semantic memory in both the verbal and non-verbal domains. SD patients often present with the complaint of word-finding difficulties. Clinical signs include fluent aphasia, anomia, impaired comprehension of word meaning, and associative visual agnosia (the inability to match semantically related pictures or objects). As the disease progresses, behavioral and personality changes are often seen similar to those seen in frontotemporal dementia although cases have been described of ‘pure’ semantic dementia with few late behavioral symptoms. Structural MRI imaging shows a characteristic pattern of atrophy in the temporal lobes (predominantly on the left), with inferior greater than superior involvement and anterior temporal lobe atrophy greater than posterior.

As another example, another form of frontotemporal dementia is Pick's disease (PiD, also PcD). A defining characteristic of the disease is build-up of tau proteins in neurons, accumulating into silver-staining, spherical aggregations known as “Pick bodies.” Symptoms include loss of speech (aphasia) and dementia. Patients with orbitofrontal dysfunction can become aggressive and socially inappropriate. They may steal or demonstrate obsessive or repetitive stereotyped behaviors. Patients with dorsomedial or dorsolateral frontal dysfunction may demonstrate a lack of concern, apathy, or decreased spontaneity. Patients can demonstrate an absence of self-monitoring, abnormal self-awareness, and an inability to appreciate meaning. Patients with gray matter loss in the bilateral posterolateral orbitofrontal cortex and right anterior insula may demonstrate changes in eating behaviors, such as a pathologic sweet tooth. Patients with more focal gray matter loss in the anterolateral orbitofrontal cortex may develop hyperphagia. While some of the symptoms can initially be alleviated, the disease progresses and patients often die within two to ten years.

E. Huntington's disease. Huntington's disease (HD) is a hereditary progressive neurodegenerative disorder characterized by the development of emotional, behavioral, and psychiatric abnormalities; loss of intellectual or cognitive functioning; and movement abnormalities (motor disturbances). The classic signs of HD include the development of chorea—involuntary, rapid, irregular, jerky movements that may affect the face, arms, legs, or trunk—as well as cognitive decline including the gradual loss of thought processing and acquired intellectual abilities. There may be impairment of memory, abstract thinking, and judgment; improper perceptions of time, place, or identity (disorientation); increased agitation; and personality changes (personality disintegration). Although symptoms typically become evident during the fourth or fifth decades of life, the age at onset is variable and ranges from early childhood to late adulthood (e.g., 70s or 80s).

HD is transmitted within families as an autosomal dominant trait. The disorder occurs as the result of abnormally long sequences or “repeats” of coded instructions within a gene on chromosome 4 (4p16.3). The progressive loss of nervous system function associated with HD results from loss of neurons in certain areas of the brain, including the basal ganglia and cerebral cortex.

F. Amyotrophic lateral sclerosis. Amyotrophic lateral sclerosis (ALS) is a rapidly progressive, invariably fatal, neurological disease that attacks motor neurons. Muscular weakness and atrophy and signs of anterior horn cell dysfunction are initially noted most often in the hands and less often in the feet. The site of onset is random, and progression is asymmetric. Cramps are common and may precede weakness. Rarely, a patient survives 30 years; 50% die within 3 years of onset, 20% live 5 years, and 10% live 10 years.

Diagnostic features include onset during middle or late adult life and progressive, generalized motor involvement without sensory abnormalities. Nerve conduction velocities are normal until late in the disease. Recent studies have documented the presentation of cognitive impairments as well, particularly a reduction in immediate verbal memory, visual memory, language, and executive function.

A decrease in cell body area, number of synapses and total synaptic length has been reported in even normal-appearing neurons of the ALS patients. It has been suggested that when the plasticity of the active zone reaches its limit, a continuing loss of synapses can lead to functional impairment. Promoting the formation or new synapses or preventing synapse loss may maintain neuron function in these patients.

G. Multiple Sclerosis. Multiple Sclerosis (MS) is characterized by various symptoms and signs of CNS dysfunction, with remissions and recurring exacerbations. The most common presenting symptoms are paresthesias in one or more extremities, in the trunk, or on one side of the face; weakness or clumsiness of a leg or hand; or visual disturbances, e.g., partial blindness and pain in one eye (retrobulbar optic neuritis), dimness of vision, or scotomas. Common cognitive impairments include impairments in memory (acquiring, retaining, and retrieving new information), attention and concentration (particularly divided attention), information processing, executive functions, visuospatial functions, and verbal fluency. Common early symptoms are ocular palsy resulting in double vision (diplopia), transient weakness of one or more extremities, slight stiffness or unusual fatigability of a limb, minor gait disturbances, difficulty with bladder control, vertigo, and mild emotional disturbances; all indicate scattered CNS involvement and often occur months or years before the disease is recognized. Excess heat may accentuate symptoms and signs.

The course is highly varied, unpredictable, and, in most patients, remittent. At first, months or years of remission may separate episodes, especially when the disease begins with retrobulbar optic neuritis. However, some patients have frequent attacks and are rapidly incapacitated; for a few the course can be rapidly progressive.

H. Glaucoma. Glaucoma is a common neurodegenerative disease that affects retinal ganglion cells (RGCs). Evidence supports the existence of compartmentalized degeneration programs in synapses and dendrites, including in RGCs. Recent evidence also indicates a correlation between cognitive impairment in older adults and glaucoma (Yochim B P, et al. Prevalence of cognitive impairment, depression, and anxiety symptoms among older adults with glaucoma. J Glaucoma. 2012; 21(4):250-254).

I. Myotonic dystrophy. Myotonic dystrophy (DM) is an autosomal dominant multisystem disorder characterized by dystrophic muscle weakness and myotonia. The molecular defect is an expanded trinucleotide (CTG) repeat in the 3′ untranslated region of the myotoninprotein kinase gene on chromosome 19q. Symptoms can occur at any age, and the range of clinical severity is broad. Myotonia is prominent in the hand muscles, and ptosis is common even in mild cases. In severe cases, marked peripheral muscular weakness occurs, often with cataracts, premature balding, hatchet facies, cardiac arrhythmias, testicular atrophy, and endocrine abnormalities (e.g., diabetes mellitus). Mental retardation is common in severe congenital forms, while an aging-related decline of frontal and temporal cognitive functions, particularly language and executive functions, is observed in milder adult forms of the disorder. Severely affected persons die by their early 50s.

J. Dementia. Dementia describes a class of disorders having symptoms affecting thinking and social abilities severely enough to interfere with daily functioning. Other instances of dementia in addition to the dementia observed in later stages of the aging-associated disorders discussed above include vascular dementia, and dementia with Lewy bodies, described below.

In vascular dementia, or “multi-infarct dementia”, cognitive impairment is caused by problems in supply of blood to the brain, typically by a series of minor strokes, or sometimes, one large stroke preceded or followed by other smaller strokes. Vascular lesions can be the result of diffuse cerebrovascular disease, such as small vessel disease, or focal lesions, or both. Patients suffering from vascular dementia present with cognitive impairment, acutely or subacutely, after an acute cerebrovascular event, after which progressive cognitive decline is observed. Cognitive impairments are similar to those observed in Alzheimer's disease, including impairments in language, memory, complex visual processing, or executive function, although the related changes in the brain are not due to AD pathology but to chronic reduced blood flow in the brain, eventually resulting in dementia. Single photon emission computed tomography (SPECT) and positron emission tomography (PET) neuroimaging may be used to confirm a diagnosis of multi-infarct dementia in conjunction with evaluations involving mental status examination.

Dementia with Lewy bodies (DLB, also known under a variety of other names including Lewy body dementia, diffuse Lewy body disease, cortical Lewy body disease, and senile dementia of Lewy type) is a type of dementia characterized anatomically by the presence of Lewy bodies (clumps of alpha-synuclein and ubiquitin protein) in neurons, detectable in post mortem brain histology. Its primary feature is cognitive decline, particularly of executive functioning. Alertness and short term memory will rise and fall.

Persistent or recurring visual hallucinations with vivid and detailed pictures are often an early diagnostic symptom. DLB it is often confused in its early stages with Alzheimer's disease and/or vascular dementia, although, where Alzheimer's disease usually begins quite gradually, DLB often has a rapid or acute onset. DLB symptoms also include motor symptoms similar to those of Parkinson's. DLB is distinguished from the dementia that sometimes occurs in Parkinson's disease by the time frame in which dementia symptoms appear relative to Parkinson symptoms. Parkinson's disease with dementia (POD) would be the diagnosis when dementia onset is more than a year after the onset of Parkinson's. DLB is diagnosed when cognitive symptoms begin at the same time or within a year of Parkinson symptoms.

K. Progressive supranuclear palsy. Progressive supranuclear palsy (PSP) is a brain disorder that causes serious and progressive problems with control of gait and balance, along with complex eye movement and thinking problems. One of the classic signs of the disease is an inability to aim the eyes properly, which occurs because of lesions in the area of the brain that coordinates eye movements. Some individuals describe this effect as a blurring. Affected individuals often show alterations of mood and behavior, including depression and apathy as well as progressive mild dementia. The disorder's long name indicates that the disease begins slowly and continues to get worse (progressive), and causes weakness (palsy) by damaging certain parts of the brain above pea-sized structures called nuclei that control eye movements (supranuclear). PSP was first described as a distinct disorder in 1964, when three scientists published a paper that distinguished the condition from Parkinson's disease. It is sometimes referred to as Steele-Richardson-Olszewski syndrome, reflecting the combined names of the scientists who defined the disorder. Although PSP gets progressively worse, no one dies from PSP itself.

L. Ataxia. People with ataxia have problems with coordination because parts of the nervous system that control movement and balance are affected. Ataxia may affect the fingers, hands, arms, legs, body, speech, and eye movements. The word ataxia is often used to describe a symptom of incoordination which can be associated with infections, injuries, other diseases, or degenerative changes in the central nervous system. Ataxia is also used to denote a group of specific degenerative diseases of the nervous system called the hereditary and sporadic ataxias which are the National Ataxia Foundation's primary emphases.

M. Multiple-system atrophy. Multiple-system atrophy (MSA) is a degenerative neurological disorder. MSA is associated with the degeneration of nerve cells in specific areas of the brain. This cell degeneration causes problems with movement, balance, and other autonomic functions of the body such as bladder control or blood-pressure regulation.

The cause of MSA is unknown and no specific risk factors have been identified. Around 55% of cases occur in men, with typical age of onset in the late 50s to early 60s. MSA often presents with some of the same symptoms as Parkinson's disease. However, MSA patients generally show minimal if any response to the dopamine medications used for Parkinson's.

N. Frailty. Frailty Syndrome (“Frailty”) is a geriatric syndrome characterized by functional and physical decline including decreased mobility, muscle weakness, physical slowness, poor endurance, low physical activity, malnourishment, and involuntary weight loss. Such decline is often accompanied and a consequence of diseases such as cognitive dysfunction and cancer. However, Frailty can occur even without disease. Individuals suffering from Frailty have an increased risk of negative prognosis from fractures, accidental falls, disability, comorbidity, and premature mortality. (C. Buigues, et al. Effect of a Prebiotic Formulation on Frailty Syndrome: A Randomized, Double-Blind Clinical Trial, Int. Mol. Sci. 2016, 17, 932). Additionally, individuals suffering from Frailty have an increased incidence of higher health care expenditure. (Id.)

Common symptoms of Frailty can be determined by certain types of tests. For example, unintentional weight loss involves a loss of at least 10 lbs. or greater than 5% of body weight in the preceding year; muscle weakness can be determined by reduced grip strength in the lowest 20% at baseline (adjusted for gender and BMI); physical slowness can be based on the time needed to walk a distance of 15 feet; poor endurance can be determined by the individual's self-reporting of exhaustion; and low physical activity can be measured using a standardized questionnaire. (Z. Palace et al., The Frailty Syndrome, Today's Geriatric Medicine 7(1), at 18 (2014)).

In some embodiments, the subject methods and compositions find use in slowing the progression of aging-associated cognitive impairment. In other words, cognitive abilities in the individual will decline more slowly following treatment by the disclosed methods than prior to or in the absence of treatment by the disclosed methods. In some such instances, the subject methods of treatment include measuring the progression of cognitive decline after treatment, and determining that the progression of cognitive decline is reduced. In some such instances, the determination is made by comparing to a reference, e.g., the rate of cognitive decline in the individual prior to treatment, e.g., as determined by measuring cognition prior at two or more time points prior to administration of the subject blood product.

The subject methods and compositions also find use in stabilizing the cognitive abilities of an individual, e.g., an individual suffering from aging-associated cognitive decline or an individual at risk of suffering from aging-associated cognitive decline. For example, the individual may demonstrate some aging-associated cognitive impairment, and progression of cognitive impairment observed prior to treatment with the disclosed methods will be halted following treatment by the disclosed methods. As another example, the individual may be at risk for developing an aging-associated cognitive decline (e.g., the individual may be aged 50 years old or older, or may have been diagnosed with an aging-associated disorder), and the cognitive abilities of the individual are substantially unchanged, i.e., no cognitive decline can be detected, following treatment by the disclosed methods as compared to prior to treatment with the disclosed methods.

The subject methods and compositions also find use in reducing cognitive impairment in an individual suffering from an aging-associated cognitive impairment. In other words, cognitive ability is improved in the individual following treatment by the subject methods. For example, the cognitive ability in the individual is increased, e.g., by 2-fold or more, 5-fold or more, 10-fold or more, 15-fold or more, 20-fold or more, 30-fold or more, or 40-fold or more, including 50-fold or more, 60-fold or more, 70-fold or more, 80-fold or more, 90-fold or more, or 100-fold or more, following treatment by the subject methods relative to the cognitive ability that is observed in the individual prior to treatment by the subject methods. In some instances, treatment by the subject methods and compositions restores the cognitive ability in the individual suffering from aging-associated cognitive decline, e.g., to their level when the individual was about 40 years old or less. In other words, cognitive impairment is abrogated.

The subject methods and plasma-comprising blood products and fractions also find use in treating unwanted conditions associated with postoperative recovery and even accelerating postoperative recovery. Such conditions and indications include, by way of example and not limitation, pain and wound healing. The subject methods and compositions of the invention also find use in treating acute and chronic pain in diseases or conditions not necessarily related to postoperative recovery. The subject methods and compositions also find use in treating wound healing that is not necessarily associated with postoperative recovery. The subject methods and compositions also find use in promoting or stimulating remyelination and treating diseases related to myelination such as multiple sclerosis.

The subject methods and plasma-comprising blood products and fractions also find use in treating indications associated with the nervous system. Such conditions, by way of example and not limitation, include central nervous system conditions such as central neuropathic pain, spinal cord injury, myelopathy, and central neuropathic pain associated with postoperative recovery. Seventeen thousand new cases of spinal injury occur per year with a prevalence of about 300,000, of which 40-75% of subjects with spinal injury having central neuropathic pain. (Jadad A et al., AHRQ Evidence Report Summaries, Agency for Healthcare Research and Quality; (1998-2005); word-wide-website: nscisc.uab.edu/Public/Facts%202016.pdf; and world-wide-website: nscisc.uab.edu/PublicDocuments/fact_figures_docs/Facts%202012%20Feb%20Final.pdf). One-third of patients experience intense pain with only ⅓ having a 50% or greater reduction in pain with treatment. (Charbonneau R, CMAJ, 189(2):E48-E49 (2017); and Hadjipavlou G, et al., BJA Education, 16(8):264-68 (2016)). Myelopathy has an occurrence rate of 605 per 1,000,000 with surgical options, but no pharmacologic treatments, indicating an unmet need in the field. (Nouri A, et al., Spine, 40(12):E675-93 (2015); The Lancet Neurology, editorial 18(7):P615 (2019)).

These conditions also include, by way of example and not limitation, plexus/nerve root conditions such as plexopathy, cervical radiculopathy, and sciatica (lumbar radiculopathy). Plexopathy has a 2-3 per 100,000 incidences. Its current options include management of neuropathic pain with antiepileptics and antidepressants, indicating an unmet need. Cervical radiculopathy's incidence is 100 per 100,000 males and 60 per 100,000 females. (McCartney S, et al., Br. J. Gen. Pract., 68(666):44-46 (2018)). Sciatica has an annual incidence of 1-5% and although many cases resolve spontaneously, sciatica becomes less responsive to treatment with prolonged duration of episodes. Treatments options include surgical procedures, standard pain medications, and steroids, indicating a need for new therapies. (Lewis R, et al., Health Technology Assessment—The Clinical Effectiveness and Cost-Effectiveness of Management Strategies for Sciatica: Systematic Review and Economic Model, No. 15.39 NIHR Journals Library (2011)).

Additional indications include peripheral nervous system disorders. These include, by way of example and not limitation: peripheral neuropathy; peripheral neuropathy associated with post-operative recovery; carpal tunnel syndrome; chemotherapy-induced peripheral neuropathy; compression and trauma; diabetic neuropathy; peripheral neuropathy associated with shingles (postherpetic neuralgia); complex regional pain syndrome; and trigeminal neuralgia. Peripheral neuropathy is a disorder of the peripheral nerves and affects at least 20 million people in the United States along. Almost 60 percent of subjects with diabetes experience diabetic neuropathy, a type of peripheral neuropathy. (word-wide-website: healthcommunities.com/neuropathy/overview-of-neuropathy.shtml). Carpal tunnel syndrome affects 3-6% of adults, and treatments include splints, steroids, and surgery. (LeBlanc K E, et al., Am Fam Physician, 83(8):952-58 (2011)). Chemotherapy-induced peripheral neuropathy occurs in 40-60% of patients both during and up to 3 months after receiving chemotherapy, with 650,000 patients reported to receive chemotherapy per year. Peripheral neuropathy leads to dose reductions in chemotherapy or even discontinuation, impacting quality of life, with no medication or supplement having been shown to prevent the disorder. (JAMA Oncology, 5(5):750, (2019)). Peripheral neuropathy related to compression and trauma occurs in 2-3% of trauma patients, with 3 million cases of trauma occurring in the United States. Although surgery is often effective, there is a need for new pharmacological agents. (American Association for the Surgery of Trauma—Trauma Facts, available at_word-wide-website: aast.org/trauma-facts; and Novak C B, Medscape—Peripheral Nerve Injuries, (Oct. 5, 2018) available at https://emedicine.medscape.com/article/1270360-overview).

Further peripheral nervous system indications that the subject methods and plasma-comprising blood products and fractions also find use in treating include diabetic neuropathy. In the United States, the population of diabetes patients is about 30 million, and 8-26% of those patients suffer from neuropathy. (Risson V, et al., Incidence and prevalence of painful diabetic neuropathy and postherpetic neuralgia in major 5 European countries, the United States and Japan, Value in Health (20):A339-A811 PSY18 (2017), available at word-wide-website:valueinhealthjournal.com/article/S1098-3015(17)31179-8/pdf). The FDA-approved options for diabetic neuropathic pain include pregabalin, duloxetine, fluoxetine, and tapentadol, all of which many patients do not respond to and none of which directly addresses nerve damage.

Peripheral neuropathy associated with shingles (postherpetic neuralgia) may also be treated by the methods and products of the invention. Twenty percent of shingles patients experience postherpetic neuralgia and there are 1 million cases per year in the United States. (See world-wide-website: emedicine.medscape.com/article/1143066-overview#a6 word-wide-website:cdc.gov/shingles/hcp/clinical-overview.html.) Gabapentin and pregabalin are approved treatments for the condition but the pain is often refractory to treatment. (Sacks G M, Am J Manag Care 19(1 Suppl):S207-13 (2013)).

Additional peripheral neuropathic indications such as complex regional pain syndrome and trigeminal neuralgia may be treated with the methods and compositions of the invention. Five and one half to twenty-six cases occur per 100,000 population. It is associated with severe pain and disability and response to treatment is variable, indicating a high unmet need. (Complex Region Pain Syndrome Fact Sheet, National Institutes of Health—National Institute of Neurological Disorders and Stroke, available at world-wide-website:ninds.nih.gov/Disorders/Patient-Caregiver-Education/Fact-Sheets/Complex-Regional-Pain-Syndrome-Fact-Sheet). Trigeminal neuralgia occurs in 4.2-28.9 per 100,000 population. It has a significant impact on quality of life, and can become resistant to treatment over time, requiring patients to try many different treatments. (Wu N, et al., J Pain, 18(Suppl 4):S69, (2017)). The only approved treatment is carbamazepine. Hence, there is an unmet need to treat the pain experienced by these patients.

Additional indications that may be treated with the methods and compositions of the invention include the following examples: central post stroke pain; central pain in multiple sclerosis; post-traumatic headaches; Dejerine-Roussy syndrome; optic neuritis; mitochondrial optic neuropathies; ischemic optic neuropathy; neuromyelitis optica; hereditary optic neuropathies; alcoholic neuropathy; Guillain-Barré Syndrome; Chronic Inflammatory Demyelinating Polyneuropathy (CIDP); Multifocal Motor Neuropathy (MNN); paraneoplastic autonomic neuropathy; peripheral neuropathy associated with sarcoidosis; peripheral neuropathy associated with rheumatoid arthritis; peripheral neuropathy associated with systemic lupus erythematosus; peripheral neuropathy associated with Sjögren's Syndrome; peripheral neuropathy associated with celiac disease; Bell's palsy; peripheral neuropathy associated with Lyme disease; peripheral neuropathy associated with leprosy; peripheral Neuropathy associated with Hepatitis B; peripheral neuropathy associated with Hepatitis C; peripheral neuropathy associated with HIV/AIDS; peripheral neuropathy associated with amyloidosis; peripheral neuropathy associated with anti-MAG; peripheral neuropathy associated with cryoglobulinemia; peripheral neuropathy associated with POEMS; toxin-Induced peripheral neuropathy; peripheral neuropathy associated with kidney disease; peripheral neuropathy associated with vasculitis; peripheral neuropathy associated with vitamin and nutrition deficiency; Charcot-Marie Tooth Disease (CMT); idiopathic peripheral neuropathy; fibromyalgia; and paraneoplastic peripheral neuropathy.

The subject methods and plasma-comprising blood products and fractions also find use in treating indications associated with wound healing. Wounds may be, for example and not as limitation, abrasions, avulsions, incisions, lacerations, and punctures. Such indications can include both chronic wounds and acute wounds. By way of example, and not limitation, wound indications include: chronic wounds such as diabetic ulcer; pressure ulcer; venous ulcer; arterial ulcer; as well as acute wounds such as surgical wounds; traumatic wounds; and burns. But any type of chronic or acute wound may be treated by the subject methods and compositions of the invention.

Diabetic ulcers affect over 2.2 million people in the United States with a global incidence of 6.4%. (Chun D, et al., J Clin Med, 8:748 (2019)). Despite several treatment options such as debridement and medical dressings, many patients endure infection and eventually require amputation, highlighting the need for new remedies, in particular pharmacological remedies.

Pressure ulcers occur at an overall rate of 1.8% of hospital admittees, with the total number of annual cases being in the hundreds of thousands. (Bauer K, et al., Ostomy Wound Manage, 62(11):30-38 (2016)). Like diabetic ulcers, treatment options such as debridement and medical dressing exist, but many patients experience infection and the ulcers can lead to mortality.

Venous ulcers occur primarily in the leg and comprise a substantial burden on the elderly and occur in about 1% of populations worldwide. (Nelzen O, Phlebolymphology, 15(4) (2008)). Venous ulcers are difficult to heal and have a significant tendency to recur than other chronic ulcers. As with diabetic and pressure ulcers, treatment options such as debridement and medical dressing exist, but their recurrence highlights a need for new treatments, in particularly pharmacological-based treatments. Arterial ulcers occur at a rate of approximately a quarter of the rate of venous ulcers. (Gabriel A, Vascular Ulcers, (2018), available at https://emedicine.medscape.com/article/1298345-overview#a6). Treatment options also include debridement and medical dressings, but there is a lack of approved pharmacological agents.

Surgical wounds occur in approximately 1.3 million patients per year. (See MediWound—Innovating Solutions for Wound & Burn Care (2019) at 19 available at http://ir.mediwound.com/static-files/cd547017-d1ed-460e-8cb2-0550b1e18a29). Surgical wounds are cuts or incisions in the skin usually made by a scalpel during surgery but can also result from a drain placed during surgery. Healing of surgical wounds is a critical outcome for surgery. Postoperative wound disruption or separation of the layers of the wound with fascial disruption can be a serious complication. (See Hospital Harm Improvement Resource—Wound Disruption (2016), available at word-wide-website:patientsafetyinstitute.ca/en/toolsResources/Hospital-Harm-Measure/Documents/Resource-Library/HHIR%20Wound %20Disruption.pdf). Additionally, healing of surgical wounds takes considerably more time in elderly patients compared to younger individuals. (Gerstein A D, Dermatol Clin, 11(4):749-57 (1993).

Traumatic wounds are primarily cuts, lacerations, puncture, or abrasion wounds with damage having been caused to the skin and the underlying tissues. Traumatic wounds are typically classified under three groups: acute wounds; cut wounds, and penetrating wounds. Acute wounds are when the skin is ripped or torn, the wound's appearance is jagged, and usually contain foreign bodies like glass, metal, gravel, sand or dirt. Cut wounds are when a sharp object penetrates the skin and underlying subcutaneous tissues. Penetrating wounds are the deepest of the three types and the most severe. Stab wounds and gunshot wounds are typical examples. (See Traumatic Wounds available at word-wide-website:woundcarecenters.org/article/wound-types/traumatic-wounds; and Leaper D J, BMJ, 332(7540):532-35 (2006)). Although there are several physical treatment options (e.g., sutures), there remains a need for pharmacological interventions.

The World Health Organization estimates that 180,000 deaths occur every year as a result of burns. And non-fatal burn injuries are a leading cause of morbidity, including prolonged hospitalization. (word-wide-website:who.int/news-room/fact-sheets/detail/burns). Typical treatment includes surgical management and dressings. Pharmacological treatment is focused on analgesia, infection control, sedation, circulating blood volume replacement, anticoagulation, and nutrition. (Green A, et al., Clinical Pharmacist, 2:249-54 (2010)). The methods and compositions of the invention can fill an unmet need for pharmacological intervention that promotes healing of the damage to the skin and underlying tissues.

The subject methods and plasma-comprising blood products and fractions can be used to treat conditions and indications associated with postoperative recovery at different time points. For example, and not as a limitation, administration to a subject can be performed: pre-operatively, perioperatively (during the procedure), or post-operatively.

One embodiment of the invention is that the subject methods and plasma-comprising blood products and fractions can be used to treat pain. Such pain, by way of example and not limitation, may include acute or chronic pain. Another embodiment of the invention is that the subject methods and plasma-comprising blood products and fractions can also be used to treat central pain or central neuropathy. Central pain includes neurological conditions caused by damage to or dysfunction of the central nervous system (CNS), including the brain, brainstem, and spinal cord. It may affect a large portion of the body or it can be restricted to specific areas. The pain may be constant or intermittent. The pain may be moderate to severe in intensity. Such pain may also be affected by touch, movement, emotions, and temperature changes. The pain may also have an immediate onset after the causative incident or may be delayed by months or years. (See Central Pain Information Page—National Institute of Neurological Disorders and Stroke, Central Pain Syndrome Information Page, available at world-wide-website:ninds.nih.gov/disorders/all-disorders/central-pain-syndrome-information-page; and Colloca L, et al., Nat Rev Dis Primers, 3:17002 (2017)). Further embodiments of the invention include using the subject methods and plasma-comprising blood productions and fractions to treat: spinal cord injury (SCI); myelopathy; plexopathy; cervical radiculopathy; sciatica (lumbar radiculopathy); central post stroke pain; central pain in multiple sclerosis; post-traumatic headaches; Dejerine-Roussy syndrome; optic neuritis; mitochondrial optic neuropathies; ischemic optic neuropathy; neuromyelitis optica; and hereditary optic neuropathies.

Another embodiment of the invention is that the subject methods and plasma-comprising blood products and fractions can also be used to treat peripheral pain or peripheral neuropathy. Peripheral neuropathy can refer to several conditions involving damage to the peripheral nervous system. More than 100 peripheral neuropathies have been identified and depend on what type(s) of nerve(s) is/are damaged including motor nerves, sensory nerves, and autonomic nerves. (See Central Page Information Page—National Institute of Neurological Disorders and Stroke, Peripheral Neuropathy Fact Sheet, available at word-wide-website:ninds.nih.gov/Disorders/Patient-Caregiver-Education/Fact-Sheets/Peripheral-Neuropathy-Fact-Sheet; and Colloca L, et al., Nat Rev Dis Primers, 3:17002 (2017)). Further embodiments of the invention include using the subject methods and plasma-comprising blood productions and fractions to treat: carpal tunnel syndrome; chemo-induced peripheral neuropathy; compression and trauma; diabetic neuropathy; peripheral neuropathy associated with Shingles (postherpetic neuralgia); complex regional pain syndrome; trigeminal neuralgia; alcoholic neuropathy; Guillain-Barré Syndrome; Chronic Inflammatory Demyelinating Polyneuropathy (CIDP); Multifocal Motor Neuropathy (MNN); paraneoplastic autonomic neuropathy; peripheral neuropathy associated with sarcoidosis; peripheral neuropathy associated with rheumatoid arthritis; peripheral neuropathy associated with systemic lupus erythematosus; peripheral neuropathy associated with Sjögren's Syndrome; peripheral neuropathy associated with celiac disease; Bell's palsy; peripheral neuropathy associated with Lyme disease; peripheral neuropathy associated with leprosy; peripheral neuropathy associated with Hepatitis B; peripheral neuropathy associated with Hepatitis C; peripheral neuropathy associated with HIV/AIDS; peripheral neuropathy associated with amyloidosis; peripheral neuropathy associated with anti-MAG; peripheral neuropathy associated with cryoglobulinemia; peripheral neuropathy associated with POEMS; Toxin-Induced peripheral neuropathy; peripheral neuropathy associated with kidney disease; peripheral neuropathy associated with vasculitis; peripheral neuropathy associated with vitamin and nutrition deficiency; Charcot-Marie Tooth Disease (CMT); idiopathic peripheral neuropathy; fibromyalgia; and paraneoplastic peripheral neuropathy.

One embodiment of the invention is that the subject methods and plasma-comprising blood products and fractions can be used to treat wounds by promoting wound healing. Further embodiments of the invention include using the subject methods and plasma-comprising blood productions and fractions to treat chronic or acute wounds. Additional embodiments of the invention include treating: diabetic ulcers; pressure ulcers; venous ulcers; arterial ulcers; surgical wounds; traumatic wounds; and burns.

### 14. Methods of Diagnosing and Monitoring for Improvement of Neurocognitive-Associated Disease

In some instances, among the variety of methods to diagnose and monitor disease progression and improvement in neurocognitive-associated disease, the following types of assessments are used alone or in combination with subjects suffering from neurodegenerative disease, as desired. The following types of methods are presented as examples and are not limited to the recited methods. Any convenient methods to monitor disease may be used in practicing the invention, as desired. Those methods are also contemplated by the methods of the invention.

A. General Cognition

Embodiments of the methods of the invention further comprise methods of monitoring the effect of a medication or treatment on a subject for treating cognitive impairment and/or age-related dementia, the method comprising comparing cognitive function before and after treatment. Those having ordinary skill in the art recognize that there are well-known methods of evaluating cognitive function. For example, and not by way of limitation, the method may comprise evaluation of cognitive function based on medical history, family history, physical and neurological examinations by clinicians who specialize dementia and cognitive function, laboratory tests, and neuropsychological assessment. Additional embodiments which are contemplated by the invention include: the assessment of consciousness, such as using the Glasgow Coma Scale (EMV); mental status examination, including the abbreviated mental test score (AMTS) or mini-mental state examination (MMSE) (Folstein et al., J. Psychiatr. Res 1975; 12:1289-198); global assessment of higher functions; estimation of intracranial pressure such as by fundoscopy.

In one embodiment, examinations of peripheral nervous system may be used to evaluate cognitive function, including any one of the followings: sense of smell, visual fields and acuity, eye movements and pupils (sympathetic and parasympathetic), sensory function of face, strength of facial and shoulder girdle muscles, hearing, taste, pharyngeal movement and reflex, tongue movements, which can be tested individually (e.g. the visual acuity can be tested by a Snellen chart; a reflex hammer used testing reflexes including masseter, biceps and triceps tendon, knee tendon, ankle jerk and plantar (i.e. Babinski sign); Muscle strength often on the MRC scale 1 to 5; Muscle tone and signs of rigidity.

### 15. Reagents, Devices, and Kits

Also provided are reagents, devices, and kits thereof for practicing one or more of the above-described methods. The subject reagents, devices, and kits thereof may vary greatly.

Reagents and devices of interest include those mentioned above with respect to the methods of preparing plasma-comprising blood product for transfusion into a subject in need hereof, for example, anti-coagulants, cryopreservatives, buffers, isotonic solutions, etc.

Kits may also comprise blood collection bags, tubing, needles, centrifugation tubes, and the like. In yet other embodiments, kits as described herein include two or more containers of blood plasma product such as plasma protein fraction, such as three or more, four or more, five or more, including six or more containers of blood plasma product. In some instances, the number of distinct containers of blood plasma product in the kit may be 9 or more, 12 or more, 15 or more, 18 or more, 21 or more, 24 or more 30 or more, including 36 or more, e.g., 48 or more. Each container may have associated therewith identifying information which includes various data about the blood plasma product contained therein, which identifying information may include one or more of the age of the donor of the blood plasma product, processing details regarding the blood plasma product, e.g., whether the plasma product was processed to remove proteins above an average molecule weight (such as described above), blood type details, etc. In some instances, each container in the kit includes identifying information about the blood plasma contained therein, and the identifying information includes information about the donor age of the blood plasma product, e.g., the identifying information provides confirming age-related data of the blood plasma product donor (where such identifying information may be the age of the donor at the time of harvest). In some instances, each container of the kit contains a blood plasma product from a donor of substantially the same age, i.e., all of the containers include product from donors that are substantially the same, if not the same, age. By substantially the same age is meant that the various donors from which the blood plasma products of the kits are obtained differ in each, in some instances, by 5 years or less, such as 4 years or less, e.g., 3 years or less, including 2 years or less, such as 1 year or less, e.g., 9 months or less, 6 months or less, 3 months or less, including 1 month or less. The identifying information can be present on any convenient component of the container, such as a label, an RFID chip, etc. The identifying information may be human readable, computer readable, etc., as desired. The containers may have any convenient configuration. While the volume of the containers may vary, in some instances the volumes range from 10 ml to 5000 mL, such as 25 mL to 2500 mL, e.g., 50 ml to 1000 mL, including 100 mL to 500 mL. The containers may be rigid or flexible, and may be fabricated from any convenient material, e.g., polymeric materials, including medical grade plastic materials. In some instances, the containers have a bag or pouch configuration. In addition to the containers, such kits may further include administration devices, e.g., as described above. The components of such kits may be provided in any suitable packaging, e.g., a box or analogous structure, configured to hold the containers and other kit components.

In addition to the above components, the subject kits will further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, portable flash drive, etc., on which the information has been recorded. Yet another means that may be present is a website address which may be used via the internet to access the information at a removed site. Any convenient means may be present in the kits.

### 16. Experimental Procedures

The following examples are put forth to provide those of ordinary skill in the art with a complete disclosure and description of how to make and use the present invention, and are not intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is degrees Centigrade, and pressure is at near atmospheric.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995; Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, and kits for genetic manipulation referred to in this disclosure are available from commercial vendors such as BioRad, Stratagene, Invitrogen, Sigma-Aldrich, and Clontech.

### Example 1—Treatment of Aging-Associate Cognitive Disorders

A. Materials and Reagents.

USP saline was purchased from Hospira (Lake Forest, Ill.). Injections were performed with 27.5 G or 30 G needles, at a volume of 150 μL per injection. Commercially-available PPF (“PPF1”) such as those Commercial PPF Preparations described above in 5% solution were stored at 4° C. Commercially-available HAS (“HAS1”) such as those Commercial HAS Preparations described above in 5% solution were stored at 4° C.

B. Animal Supply and Husbandry.

The mouse strains NOD.CB17-Prkdcscid/NcrCrl (“NODscid,” Strain Code 394, Charles River, Mass.) (Bosma, M. et al., The scid mouse mutant. 137 Curr Top Microbiol Immunol 197 (1988)) and NOD scid gamma (“NSG,” Strain Code 005557, The Jackson Laboratory, Bar Harbor, Me.) were used. Each mouse was ear punched to designate a unique identification number. All mice were individually housed under specific pathogen-free conditions under a 12-hour light, 12-hour dark cycle, and all animal handling and use was in accordance with IACUC approved standard guidelines.

C. Administration.

Unless described differently below, NSG and NODscid mice were injected with USP saline, 5% PPF1, or 5% HAS1 twice weekly via intravenous tail vein injection (150 μL per injection) for up to 6 months.

D. Open Field

Open field tests were utilized to determine exploratory behavior of the subject mice. The open field test is an empty test arena, usually round or square. The mouse is placed inside a 50 cm×50 cm open filed arena for 15 minutes and the level of the mouse's activity is measured. Rearing time was measured by tracking the duration the forepaws were on the walls of the box. Total distance covered and velocity was also measured for duration of the test. CleverSys TopScan V3.0 (Reston, Va.) was used to track mouse behavior in open field. Open field chambers were constructed by CleverSys.

E. Y-Maze

Mice were allowed to explore two arms of a Y-maze (start+familiar) for 5 minutes. One hour later, mice were allowed to explore all three arms, and total time and number of entries in the arms were recorded.

F. Barnes Maze

Mice were trained on four consecutive days in a modified Barnes maze and given a maximum of 120 seconds to find the escape hole. (See Barnes, C. A., Memory deficits associated with senescence: A neurophysiological and behavioral study in the rat, J. COMPARATIVE AND PHYSIOLOGICAL PSYCHOLOGY, 93(1): 74-104 (1979); and for the modified maze, Faizi, M. et al., Thy1-hAPP(Lond/Swe+) mouse model of Alzheimer's disease displays broad behavioral deficits in sensorimotor, cognitive and social function, BRAIN BEHAV. 2(2): 142-54, (2012)). The escape hole remained the same for four trials on a training day, but changed between training days. The latency to the escape hole was recorded for each mouse cohort on four separate training days.

G. DCX- and Ki67-Positive Cells

Doublecortin (DCX) is a microtubule-associated protein that is expressed by neuronal precursor cells. It is also expressed by immature neurons in embryonic and adult cortical structures. When they are actively dividing, neuronal precursor cells express DCX. The protein downregulates after two weeks. Because of this association, it is useful as a marker of neurogenesis.

Brain tissue processing and immunohistochemistry was performed on free-floating sections well-described techniques (Luo, J. et al. Glia-dependent TGF-b signaling, acting independently of the TH17 pathway, is critical for initiation of murine autoimmune encephalomyelitis. J. CLIN. INVEST. 117, 3306-3315 (2007)). Mice were anesthetized and perfused with 0.9% saline. Brains were removed and subsequently fixed with phosphate-buffered 4% paraformaldehyde, pH 7.4, at 4° C. before sunk through 30% sucrose for cryoprotection. Brains were subsequently sectioned at 30 μm with a cryomicrotome at −22° C. Sections were stored in cyroprotective medium. The primary antibody used was goat anti-Dcx (Santa Cruz Biotechnology at 1:500 for twice weekly dosing experiments or 1:200 in the three times per week dosing experiments) or rabbit anti-Ki67 (1:500 Abcam). Primary antibody staining was revealed using biotinylated secondary antibodies and the ABCkit (Vector) with diaminobenzidine (DAB, Sigma-Aldrich) or fluorescence-conjugated secondary antibodies. To estimate the total number of Dcx-positive cells per dentate gyrus, immunopositive cells in the granule cell and subgranular cell layer of the dentate gyrus were counted in three coronal hemibrain sections through the hippocampus and averaged.

H. Barnes Maze Test on Aged NSG Mice Treated with Young Plasma, Effluent I, or Effluent II/III

Aged NSG mice (aged 12 months), were separated into several groups (all of n=14), and received 150 μL saline, young plasma, Effluent I, or Effluent II/III by tail vein injection prior to initiation of behavior tests. Each separate group was separated into 3 cohorts with each cohort initiated with behavior tests on a different week.

I. Barnes Maze and Cell Survival (BrdU Staining) in Aged NSG Mice Treated Three Times Per Week with Young Plasma or PPF1

Aged (12 months) male NSG mice were treated intravenously via tail vein injection with 150 μL of clarified young human plasma (young plasma), PPF1, or saline three times per week for four weeks. The regimen was changed to twice per week during weeks 5 and 6, which were the behavioral testing weeks.

Prior to treatment, the mice were divided into three cohorts of 13-15 mice each. Each cohort underwent five days of BrdU injections intraperitoneally (i.p.) prior to the start of treatment of young plasma, PPF1, or saline as described above.

During weeks 5 and 6, behavioral testing was performed, and latency to target hole was measured for each mouse in Barnes Maze testing. Each testing session lasted for a maximum of 120 seconds. The event of finding the target hole was recorded using software that determined when the nose of the mouse entered the area defined as the target hole.

At the end of behavioral testing, the animals were sacrificed, and six sections per hippocampus were quantified using brightfield microscope to determine presence of BrdU positive cells within the granule cell layer of the dentate gyrus. As representative sections throughout the different regions of the hippocampus, the average number of BrdU positive cells were multiplied by 72, which was the total number of sections for each animal's hippocampus, in order to give an estimate of the total number of BrdU positive cells.

J. Neurosphere and Cortex Culture Assays

### 1. Tuj1 and DAPI Staining

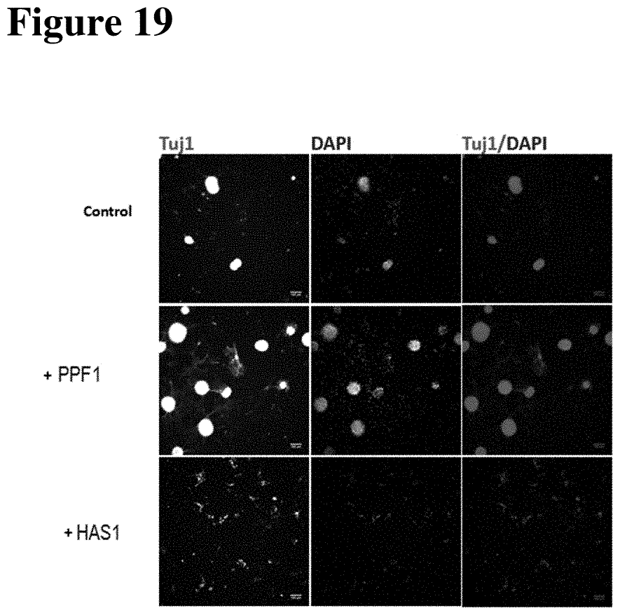

Mouse C57 E14,15 cortices (Lonza: M-CX-300) were suspended in 12 ml of neural basal media supplemented with B27, 2 mM Glutamax (Sigma-Aldrich). 200 μL was added to each well of a 96-well plate pre-coated with collagen I (Corning, Inc.). After 16 hours, plating media was replaced with pre-warmed (37° C.) control media (Neural basal media with B27, 2 mM Glutamax (Gibco). On day 4 in vitro (“days in vitro”, or “DIV”), culture media was replaced with fresh control media, control media and 10% PPF1, control media and 10% HAS1, vehicle and 10% PPF1, or vehicle and 10% HAS1. Cultures were maintained for 21 days with 75% of media changed to fresh media every 3 days. At 21 DIV, cultures were washed 3× with PBS then fixed with 4% Paraformaldehyde for 20 minutes at room temperature (RT). After fixation, cultures were washed 2× with PBS then permeabilized with 0.1% Triton X100 for 5-20 minutes. After permeabilization, cultures were blocked with 3% bovine serum albumin (Sigma-Aldrich) for 60 minutes at RT. After 60 minutes, blocking solution was aspirated and cultures were labeled with anti-Tuj1 antibody (AbCam-1:500) at 4° C. overnight. After labeling, cultures were washed 3× with PBS+0.1% BSA then stained with A647-conjugated Donkey anti-mouse antibody at 4° C. overnight (1:1000). Cultures were then washed 2× with PBS and labeled with Hoechst 33342 (1:1000) for 20 min. Samples were washed 3× with PBS after Hoechst labeling. Twenty-five (25) fields were acquired for each well using 10× magnification using GE InCell Analyzer 2000 (GE Healthcare Life Sciences). Results are shown in FIG. 19.

### 2. Net Neurite Length

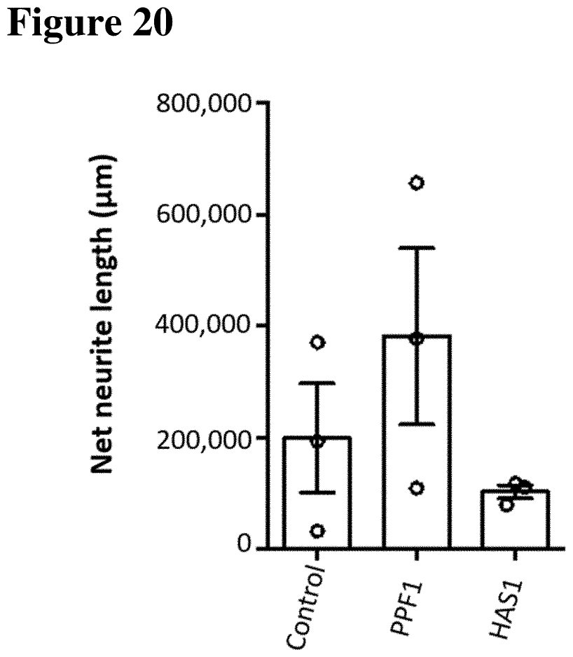

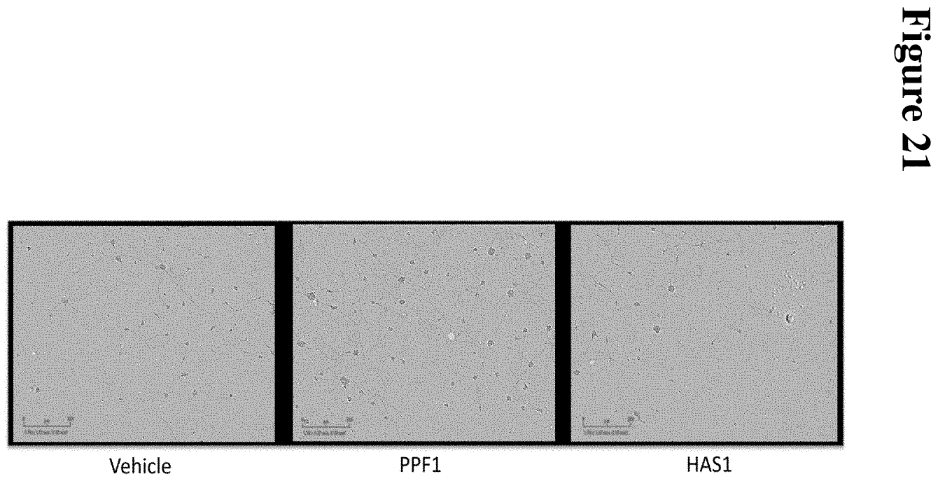

Net neurite length was determined from cultures as described in the previous section. Neurite analysis was performed using a custom algorithm generated by GE InCell Investigator Developer Toolbox. Results from control and vehicle treated samples were nearly identical therefore were combined for statistical analysis. Results are shown in FIG. 20.

### 3. Cortex Culture Sphere Number and Size; Process Length and Branching

Mouse C57 E14,15 cortices (Lonza: M-CX-300) were suspended in 12 mL of neurobasal media supplemented with B27, 2 mM Glutamax (Sigma-Aldrich). 200 μL was added to each well of a 96-well plate pre-coated with polylysine and laminin. Four days later, 50% of the media was exchanged with fresh media and treated with test article (vehicle, PPF1, or HAS1) to a final concentration of 10%. This was repeated three days later. On Day 7 of treatment, cells were imaged in phase contrast at 10× magnification with IncuCyte (Ann Arbor, Mich.) and analyzed with standard “Neurite and Cell-Body” algorithms. Six replicates were analyzed with four images taken per replicate. Standard error is displayed. Significance is shown for 2 tailed T-test as P<0.5. Results are displayed in FIGS. 21 and 22.

### 4. Sox2 Neurosphere Staining

Mouse C57 E14,15 cortical neurons (Lonza: M-CX-300) were suspended in neurobasal media supplemented with B27, 2 mM Glutamax (Sigma-Aldrich) at 100-200K cells/ml. 200 μL was added to each well of a 96-well plate pre-coated with collagen I (Corning, Inc.). After 16 hours, plating media was replaced with pre-warmed (37° C.) control media (Neurobasal media with B27, 2 mM Glutamax (Gibco)). On day 4 in vitro (“days in vitro”, or “DIV”), culture media was replaced with fresh control media, control media with HAS vehicle (vehicle), control media and 10% PPF1, control media and 10% HAS1. Cultures were maintained for 21 days with 75% of media changed to fresh media every 3-4 days. At 21 DIV, cultures were washed 3× with PBS then fixed with 4% Paraformaldehyde for 20 minutes at room temperature (RT). After fixation, cultures were washed 2× with PBS then permeabilized with 0.1% Triton 100× for 5-20 minutes. After permeabilization, cultures were blocked with 3% bovine serum albumin (Sigma-Aldrich) for 60 minutes at RT. After 60 minutes, blocking solution was aspirated and cultures were labeled with anti-Tuj1 antibody (AbCam-1:500) and Rabbit anti SOX2 (AbCam: 1:5000 at 4° C. overnight. After labeling, cultures were washed 3× with PBS+0.1% BSA then stained with Donkey anti-mouse-647 (AbCam) and Sheep anti-rabbit-Texas Red at 4° C. overnight (1:1000). Cultures were then washed 2× with PBS and labeled with Hoechst (1:1000) for 20 min. Samples were washed 3× with PBS after Hoechst labeling. Twenty-five (20 or 25) fields were acquired for each well using 10× magnification of InCell Analyzer 2000 (GE Healthcare Life Sciences). Neurosphere and neurite analysis were done using custom algorithm generated by GE InCell Investigator Developer Toolbox. Results from control and vehicle treated samples were nearly identical therefore were combined for statistical analysis. Results are displayed in FIG. 23.

K. Results of In Vivo Experiments

### 1. Open Field Test with 3-Month and 13-Month-Old NSG Mice

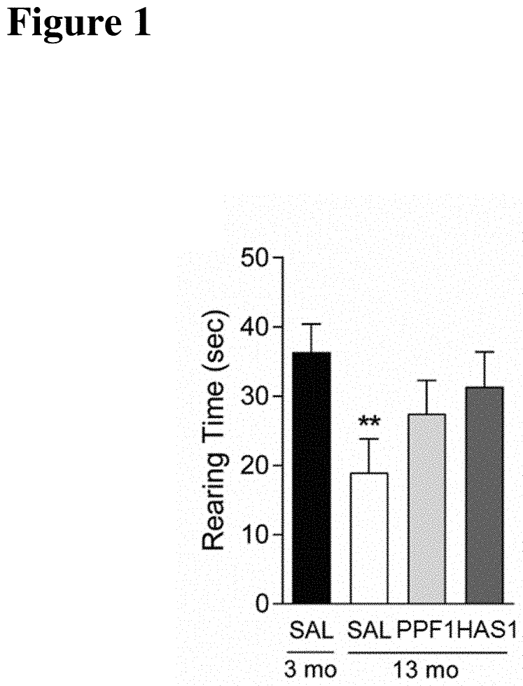

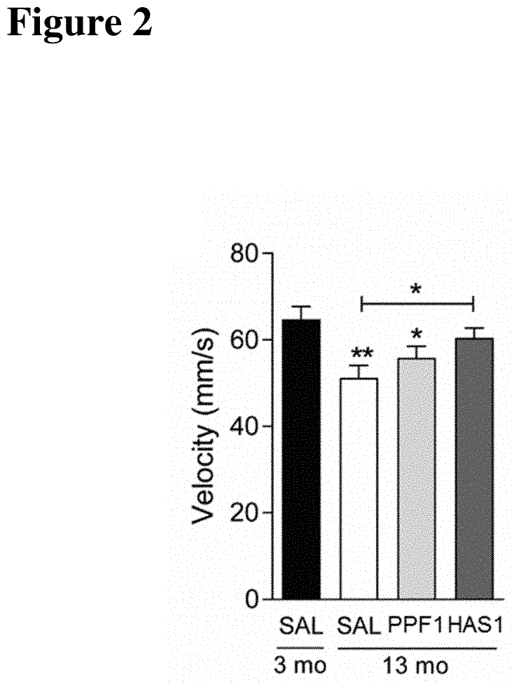

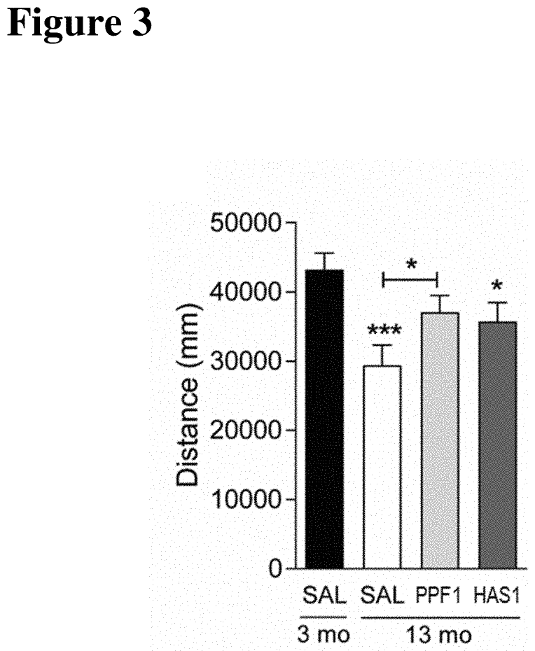

3-month (young) or 13-month-old (old) NSG mice were placed in an Open Field chamber for 15 minutes. The time spent rearing FIG. 1, velocity FIG. 2, and distance FIG. 3 were measured. FIG. 1 shows that 13-month-old mice spent less time rearing than 3-month-old mice, but that PPF1 and HAS1-treated mice were not significantly different from young mice. FIG. 2 shows that saline (control) and PPF1-treated 13-month-old mice were significantly slower than 3-month-old mice. However, HAS1-treated mice were significantly faster than saline-treated mice, and not significantly different from young mice. FIG. 3 shows that saline (control) and HAS1-treated old mice had less locomotor activity than young mice, and PPF1-treated mice covered more distance than saline-treated mice. All data shown are mean±s.e.m; *P<0.05; **P<0.01; ***P<0.001; t-test; n=20, 18, 18, 19. (SAL=saline).

### 2. Y-Maze Test with 3-Month and 13-Month-Old NSG Mice

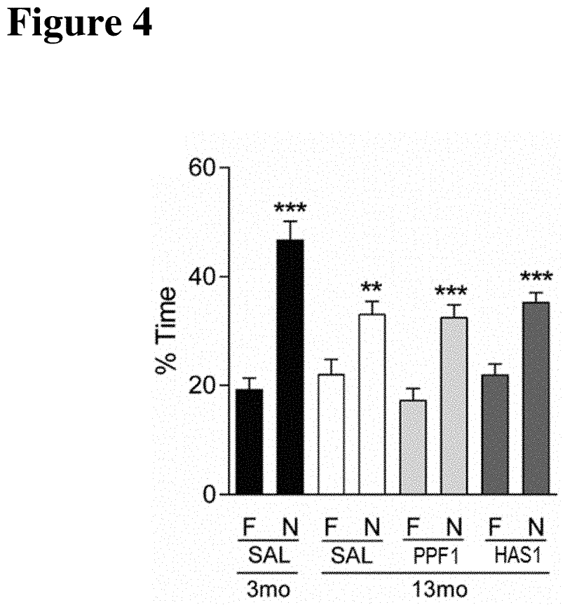

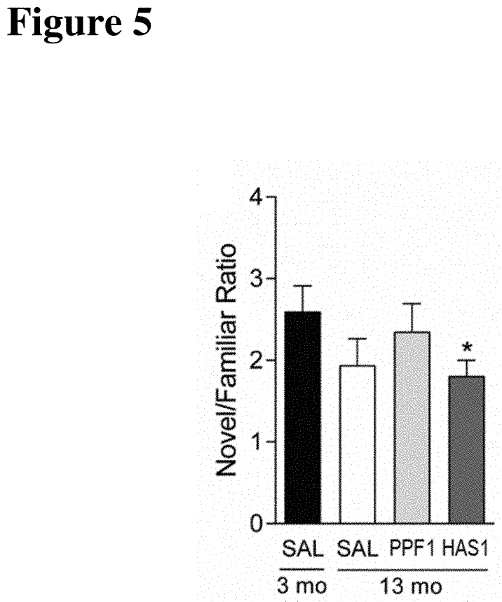

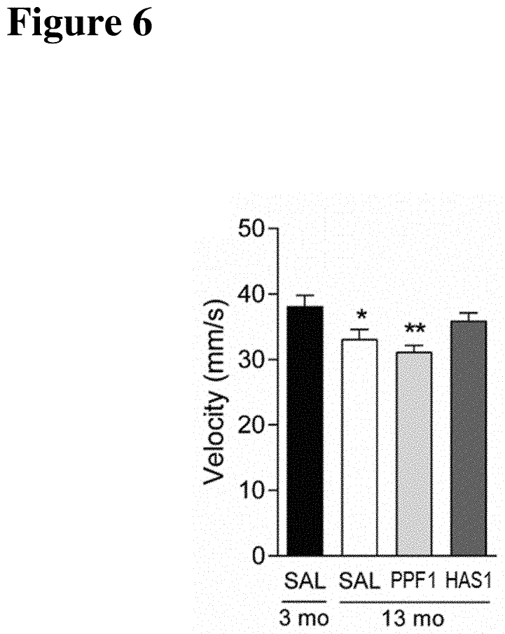

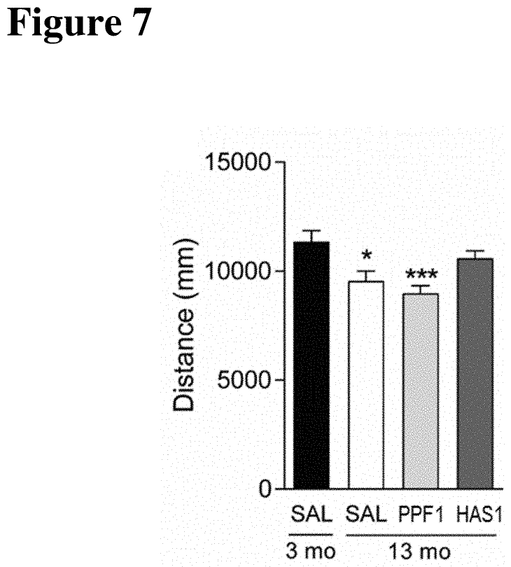

Young (3-month-old) and old (13-month-old) NSG mice were tested in the cued Y-maze as a test for memory. FIG. 4 shows that all mice spent significantly more time in the novel (N) arm than the familiar (F) arm. FIG. 5 shows that HAS1-treated old mice were significantly impaired in their memory for the familiar arm compared to young mice, whereas PPF1-treated mice trended towards improved memory for the familiar arm. FIG. 6 shows that saline and PPF1-treated, but not HAS1-treated old mice, were significantly slower than young mice. FIG. 7 shows that saline and PPF1-treated, but not HAS1-treated old mice, covered less distance than young mice. All data shown are mean±s.e.m; *P<0.05; **P<0.01; ***P<0.001; Paired t-test; n=20, 18, 18, 19. (SAL=saline).

### 3. Fear Conditioning Test for Memory with 3-Month and 13-Month-Old NSG Mice

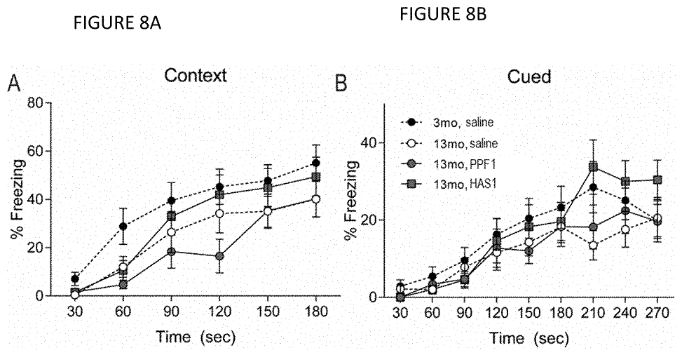

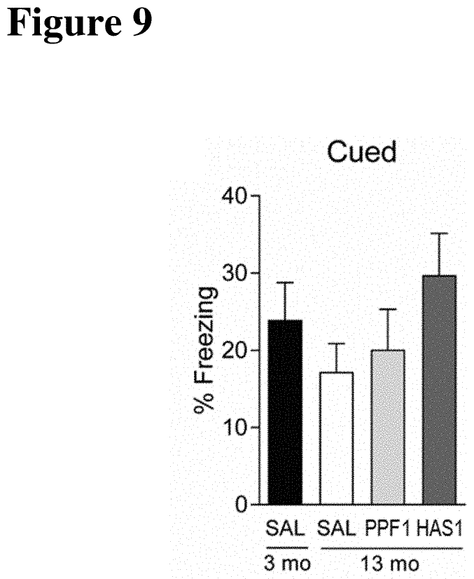

Young (3-month-old) and old (13-month-old) NSG mice were tested in the fear conditioning test for memory. FIG. 8A shows that 13-month-old mice trended to spend less time freezing than 3-month-old mice, whereas HAS1-treated mice spent almost as much time freezing as 3-month-old mice. FIG. 8B shows that in the cued test for memory of the auditory cue, 13-month-old control-treated mice performed the worst and froze the least amount of time. HAS1-treated mice trended to spend more time freezing, indicating improved memory for the tone. FIG. 9 shows the quantification of the last 90 seconds of the cued test for memory and shows that HAS1-treated mice trended to spend more time freezing, indicating improved memory. n=20, 16, 17, 19. (SAL=saline).

### 4. Barnes Maze Test for Spatial Memory with 3-Month and 13-Month-Old NSG Mice

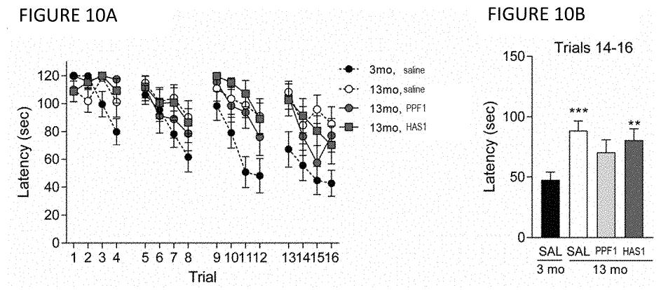

Young (3-month-old) and old (13-month-old) NSG mice were tested in the Barnes maze test for spatial memory. FIG. 10A shows that 3-month-old mice performed the best and had the fastest latency to reach the target hole by the last trial. FIG. 10B shows the quantification of the average of the last 3 trials which demonstrates that saline- and HAS1-treated old mice were significantly impaired in their memory of the target hole compared to young mice, but the PPF1-treated mice were not significantly different from young mice. **P<0.01; ***P<0.001; Unpaired t-test; n=20, 18, 18, 19. (SAL=saline).

### 5. Immunostaining with 3-Month and 13-Month-Old NSG Mice

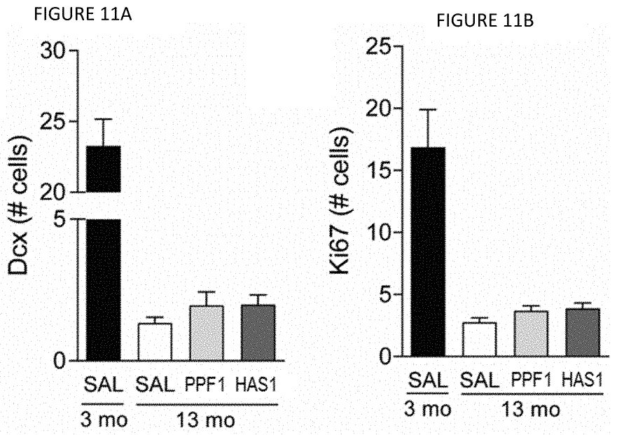

Brain sections were stained for doublecortin (Dcx), a marker for newborn neurons or for Ki67, a marker for proliferating cells in 3-month and 13-month-old NSG mice treated twice weekly with saline, PPF1, or HAS1. Dcx- and Ki67-positive cells were counted in the dentate gyrus of young and old NSG mice. FIGS. 11A and 11B respectively show that all old mice had dramatically lower numbers of Dcx- or Ki67-positive cells. PPF1 and HAS1-treated mice trended towards increased numbers of Dcx- and Ki67-positive cells compared to saline-treated mice.

### 6. Immunostaining with 3-Month and 13-Month-Old NSG Mice Treated Three Times Weekly with PPF1 and HAS1

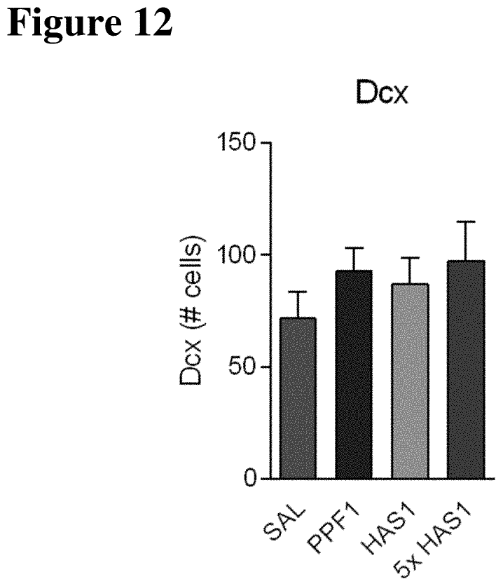

Brain sections were stained for doublecortin (Dcx), a marker for newborn neurons or for Ki67, a marker for proliferating cells in 13-month-old mice. The mice were treated three times per week with saline, PPF1, 1× concentrated HAS1, or 5× concentrated HAS1. Dcx- and Ki67-positive cells were counted in the dentate gyrus. FIG. 12 shows that mice treated with PPF1 trended towards an increase in neurogenesis (as indicated by Dcx staining), compared to saline control treated animals. Also shown is that more concentrated HAS1 trended towards increased neurogenesis compared to saline-treated animals.

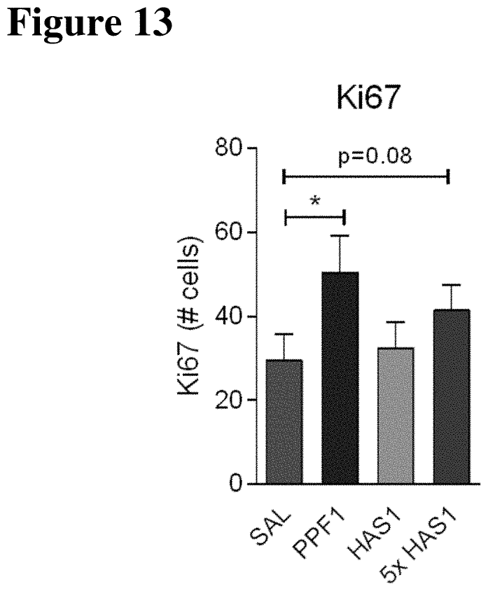

FIG. 13 shows that mice treated with PPF1 had a significant increase in cell proliferation (as indicated by Ki67 staining), compared to saline control treated animals. Also shown is that more concentrated HAS1 trended towards increased neurogenesis compared to saline-treated animals. *P<0.05; unpaired t-test against saline group; all data shown are mean±s.e.m.

### 7. Open Field Test with NODscid Mice

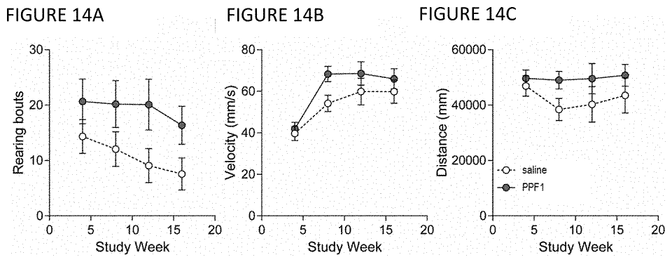

NODscid mice were treated twice weekly via intravenous tail vein injection with either saline or PPF1 starting at 6 months of age. The starting number of mice were 20 for each group. Mice were placed in the Open Field chamber for 15 minutes and locomotor activity was recorded. FIG. 14A shows that PPF1-treated mice trend towards increased rearing activity compared to saline-treated mice. FIGS. 14B and 14C respectively show that PPF1-treated mice also trend towards improved velocity and distance covered compared to saline-treated mice.

### 8. Barnes Maze with Aged (12-Month-Old) NSG Mice Treated with Young Plasma, Effluent I, and Effluent II/III

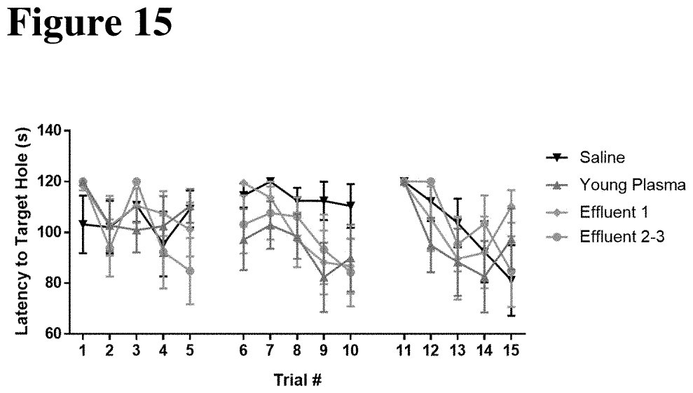

Aged NSG mice (aged 12 months), were separated into several groups (all of size n=14), and received 150 μL saline, young plasma, Effluent I, or Effluent II/III by tail vein injection prior to initiation of behavior tests. Each separate group was further separated into 3 cohorts with each cohort initiated with behavior tests on a different week. Mice were tested in a modified Barnes Maze (as described above) to assess spatial learning and memory. FIG. 15 shows that treatment with young plasma, Effluent I, or Effluent II/III trended towards significant improvement in latency for aged NSG mice to reach the target hole.

### 9. Barnes Maze and Cell Survival with Aged NSG Mice Treated with Young Plasma and PPF1

As described above, aged male NSG mice (aged 12 months) were treated with 150 μL of clarified young human plasma (young plasma), PPF1, or saline three times per week (i.v.) for 4 weeks, and then twice per week during weeks 5 and 6, which were the weeks in which testing was performed is reported.

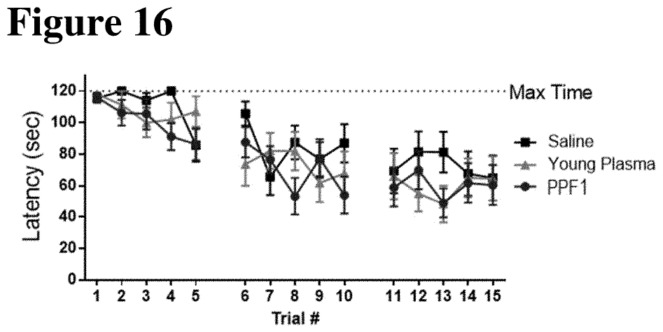

FIG. 16 reports the latency to reach the Barnes Maze hole for each treatment cohort. Treatment with PPF1 significantly improved spatial memory in aged mice compared to control, while treatment with young plasma trended towards improved spatial memory compared to control. (n: Saline=12, PPF1=14, young plasma=11). *P<0.05; mean±s.e.m.; unpaired T-Test.

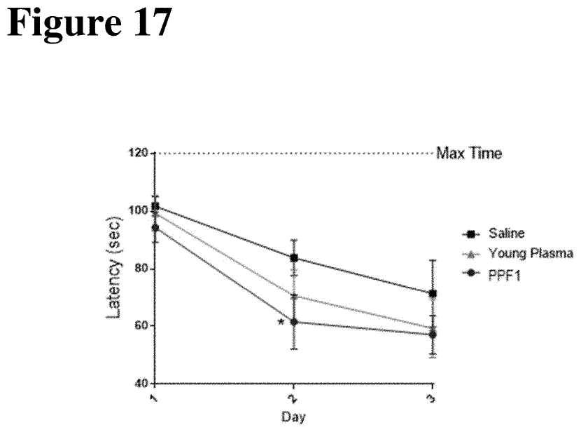

FIG. 17 reports the average latency to find the target hole for the last three trials for each day of testing. Again, treatment with PPF1 significantly improved spatial memory in aged mice compared to control, while treatment with young plasma trended towards improved spatial memory compared to control. *P<0.05; mean±s.e.m.; unpaired T-Test.

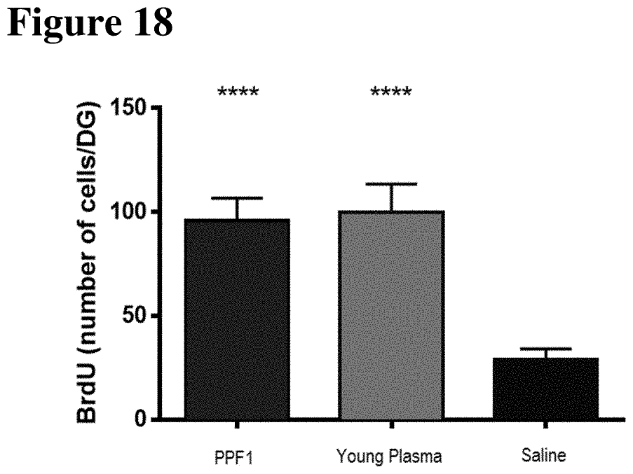

FIG. 18 reports the effect of young human plasma and PPF1 on cell survival as determined by number of BrdU positively-labeled cells (i.e. proliferating cells) within the granule layer of the dentate gyrus of aged (12 months) NSG mice. BrdU was administered for five days (i.p.) prior to commencing the intravenous injections of young plasma, PPF1, or saline control as described above. A significant increase in cell survival was observed in both young human plasma and PPF1-treated mice compared to saline control. Statistical significance was determined using One-Way ANOVA with Dunnett's multiple comparison post-hoc analysis between PPF1 and young human plasma compared to saline treatment. (n: Saline=13; PPF1=13; young plasma=11, ****P>0.0001, Unpaired T-Test between PPF1 or young human plasma and saline treatment).

L. Results of In Vitro Neurosphere and Cortex Culture Assays

FIG. 19 shows that PPF1 and HAS1 differentially modulate neurosphere proliferation in cortex culture. Cortices from E14-15 C57 mice were cultured on collagen I-coated 96-well plates in culture media containing vehicle alone, PPF1 (10%), or HAS1 (10%). Example images of neurospheres from cortical cultures after 21 days in vitro, imaged for Tuj1 (neuron-specific class III beta-tubulin), DAPI (4′,6-diamidino-2-phenylindole), or both TuJ1 and DAPI are shown. FIG. 19 shows that PPF1 increases the amount of neurospheres which express either Tuj1 or DAPI. The increase in Tuj1 expression demonstrates that PPF1-treated cortical cultures produce more neurospheres which have differentiated into a more neuronal-like phenotype.

FIG. 20 depicts three cultures of C57 mouse E14-15 cortical neurons (Lonza: M-CX-300) suspended in neurobasal media supplemented with B27, 2 mM Glutamax (Sigma-Aldrich) at 100-200K cells/mL, coated on collagen I-coated 96-well plates in culture media containing vehicle, PPF1 (10%), or HAS1 (10%). Net neurite length, indicative of neurogenesis, occurred in PPF1-treated cultures compared to control or HAS1-treated cultures.

FIG. 21 depicts three cultures of C57 mouse E14-15 cortical neurons (Lonza: M-CX-300) suspended in neurobasal media supplemented with B27, 2 mM Glutamax (Sigma-Aldrich) at 100-200K cells/mL, coated on collagen I-coated 96-well plates in culture media containing vehicle, PPF1 (10%), or HAS1 (10%). An IncuCyte software algorithm available from Essen BioSciences (Ann Arbor, Mich.) detected cortex culture spheres (highlighted in yellow) and processes (highlighted in pink). More spheres and processes were observed in PPF1-treated cultures and increased sphere size and process branching was also observed in PPF1-treated cultures. The scale bars are 300 μm each.

FIG. 22. FIGS. 22A-D report the number of spheres, the process length, process branch points, and sphere size, respectively. Quantification was performed using an IncuCyte software algorithm available from Essen BioSciences (Ann Arbor, Mich.). Standard error is displayed. Significance is shown using a 2-tailed T-Test. FIG. 22A shows that PPF1-treated cultures have an increased number of spheres compared to vehicle or HAS1-treated cultures. (P=0.0006, PPF1 vs. vehicle; P=0.0007, PPF1 vs. HAS1). FIG. 22B shows that PPF1-treated cultures display increased process length compared to vehicle or HAS1-treated cultures. (P=4e⁻⁸, PPF1 vs. vehicle; P=0.002, PPF1 vs. HAS1; and P=0.018, HAS1 vs. vehicle). FIG. 22C shows that PPF1-treated cultures produce more process branch points compared to vehicle or HAS1-treated cultures. (P=0.002 PPF1 vs. vehicle; P=0.004, PPF1 vs. HAS1). FIG. 22D shows that PPF1-treated cultures are associated with increased sphere size compared to vehicle or HAS1-treated cultures. (P=0.002 PPF1 vs. vehicle; P=0.004, PPF1 vs. HAS1). Together, the results of this data indicate that PPF1 (and HAS1 to a less significant degree) treatment are associated with characteristics indicative of increased cortex culture cellular growth and process formation.

FIG. 23 displays the number of neurospheres staining positive for Sox2, a transcription factor which plays an important role in maintaining embryonic and neural stem cells. Quantification was performed using a GE InCell Investigator Toolbox algorithm. PPF1-treated cultures produced a significantly increased number of neurospheres staining positive for Sox2, indicating that PPF1 treatment is associated with an increase in number of cells with the potential for neurogenesis.

### Example 2—Improvement of Pain and Postoperative Recovery

### 1. Models for Pain

a) Pain—Treatment Before Injury

(1) Alteration of Neuropathic Nerve Injury

A chronic pain model employing chronic constrictive injury (CCI) was used to determine levels of pain experienced by 22-month-old C57BL/6J mice treated with: (1) PPF1 following CCI; (2) vehicle following CCI; or (3) vehicle following sham surgery. Using such a model, the nervous system becomes regulated to a persistent state of high reactivity which lowers the pain threshold long after the initial injury has occurred. (See, e.g., Safakhah, H. A. et. al., Journal of Pain, 10:1457-66 and Suter M R, et al., Anesthesiology Res and Practice (2011) which are herein incorporated by reference in their entirety).

PPF1 is a PPF with approximately 88% normal human albumin (in relation to total protein), 12% alpha and beta globulins, and no more than 1% gamma globulin as determined by electrophoresis. Except where noted, PPF1 is administered in the examples herein in vivo using a 5% solution (w/v, 50 g/L). PPF2 is also a PPF, but a different lot from PPF1. PPF2 meets the same protein content and concentration specifications as PPF1.

FIG. 24 depicts timeline of a CCI experiment. Twenty-three-month-old wild type mice were administered a CCI or sham surgery via ligation 24 hours prior to administration of a 7-consecutive-day pulse dosing regimen of 150 uL/day (intravenously tail-vein) of either PPF1 or vehicle control. Behavior was assessed during week four, and tissue collection for histology occurred at week five.

FIG. 25 is a representation depicting the location of the CCI administered to twenty-three-month-old wild type mice. The ligation was administered on the sciatic nerve as indicated by the figure. The figure was adapted from Suter M R, et al., Anesthesiology Res and Practice, (2011), which is incorporated herein by reference in its entirety.

FIG. 26 reports data from a mechanical von Frey allodynia test in wild-type mice 4 weeks after CCI or sham surgery as detailed in FIG. 24. To determine an animal's tolerance to mechanical pressure, the hind paw enervated by the subject sciatic nerve, was stimulated by differing thicknesses of von Frey filaments. The pressure at which the mouse withdrew its hind paw was measured and plotted in FIG. 26. The figure illustrates that mice treated with PPF1 after CCI exhibited significantly less pain (could withstand more pressure) than those treated with vehicle control after CCI. Sham surgery animals also exhibited significantly less pain that those treated with vehicle control after CCI. The primary finding is that PPF1 has a positive effect on mechanical nociception deficits induced by CCI. ***P<0.001 CCI treated with PPF1 vs. CCI Vehicle treatment, *P<0.05 Sham vehicle vs. CCI vehicle; One-way ANOVA with Tukey post-hoc analysis.

FIG. 27 reports data from hippocampal histology performed on the wild type mice described in FIG. 24. Neurogenesis was measured using the doublecortin (DCX) marker. Mice who received CCI surgery and were treated with PPF1 had significantly increased neurogenesis in the dentate gyrus of the hippocampus than those who received vehicle. Mice who received sham operation trended towards greater neurogenesis than mice who received CCI surgery, both groups received vehicle treatment post-surgery. Thus, PPF1 exhibited the ability to restore neurogenesis after chronic nerve injury. *P<0.05 CCI treated with PPF1 vs. CCI Vehicle treatment; Unpaired T-Test.

FIG. 28 reports data from hippocampal histology performed on the wild type mice described in FIG. 24. Inflammatory marker as measured by CD68 expression was quantified. Our findings illustrate that mice which received CCI surgery and vehicle treatment expressed a significantly greater number of CD68 positive cells in the hippocampus than those were treated with PPF1 following CCI surgery. PPF1 treated animals had similar inflammation levels to that of the sham surgery group. This illustrates that PPF1 can help to ameliorate neuroinflammation resulting from chronic nerve injury. *P<0.05 CCI treated with PPF1 vs. CCI Vehicle treatment, Sham vehicle vs. CCI vehicle; One-way ANOVA with Tukey post-hoc analysis.

FIG. 29 reports data from a mechanical von Frey allodynia test in C57BL/6J mice which received CCI or sham surgery and tested in a timeline as described in FIG. 24. Twenty-two-month-old mice were administered a 7-consecutive-day pulse dosing regimen of 150 uL/day (intravenous tail-vein) of either PPF1 or vehicle control. Another group received Gabapentin at 75 mg/kg (intraperitoneal administration) daily for 7 consecutive days. All treatments were initiated 24 hours after CCI or sham surgery. To determine an animal's tolerance to mechanical pressure, the hind paw enervated by the subject sciatic nerve, was stimulated by differing thicknesses of von Frey filaments. The pressure at which the mouse withdrew its hind paw was assessed and represented in FIG. 29 as weeks post CCI or sham surgery. The figure illustrates that mice administered PPF1 following CCI surgery had significantly increased tolerance to mechanical nociception at all assessed timepoints than those treated with vehicle after CCI. Conversely, mice administered Gabapentin only show significant improvement in mechanical nociception at 2 weeks following CCI surgery and are similar to vehicle treated mice at all other timepoints. Sham surgery mice show significantly increased response to mechanical nociception at 3 and 5 weeks following surgical manipulation. Together, these data illustrate that PPF1 ameliorates peripheral pain for a greater amount of time than that of standard of care treatments (Gabapentin). ***, ****P<0.001, P<0.0001 PPF1 vs. Vehicle control; ANOVA with Tukey Post-hoc analysis. *P<0.05 Gabapentin vs. Vehicle control; ANOVA with Tukey Post-hoc analysis. *, **P<0.05, P<0.01 Sham vs. Vehicle control; ANOVA with Tukey Post-hoc analysis.

FIG. 30 reports data from a hot plate test on wild-type mice treated as described in FIG. 24 and as described by Woolfe and Macdonald. (Woolfe G. and Macdonald A D, J. Pharmacol. Exp. Ther. 80:300-07 (1944), which is incorporated by reference herein in its entirety). The hot plate is set to a temperature of 55° C. Mice are acclimated to being placed inside a clear cylinder for 30 minutes. The cylinder is placed upon the hot plate and a timer started. When nocifensive behaviors (e.g. hind paw licking or jumping) are first observed, the time is recorded as latency. If no nocifensive behaviors are observed, the animal is removed at a pre-determined cut-off time such as 30 seconds to prevent tissue damage. Mice are only tested at 2- and 5-weeks post CCI surgery, as repetitive exposure to testing has been shown to alter sensitivity. FIG. 30 illustrates hot plate nocifensive latency 5 weeks after CCI or sham surgery. PPF1 treatment are significantly less sensitive to hot plate stimuli compared to mice given CCI plus vehicle control, indicating a rescue effect by PPF1. **P<0.01 Sham vs. CCI surgery, ****P<0.0001 PPF1 vs. Vehicle treated CCI surgery mice. ANOVA with Tukey Post-hoc analysis.

(2) Prevention of Neuroinflammation in the Spinal Cord

A separate study similar to the preceding study (above) was performed on 22-month-old C57BL/6J mice. Cohorts of mice were treated as follows: (1) PPF (PPF2) following CCI; (2) vehicle following CCI; (3) recombinant human albumin (rhAlb) following CCI; or (4) vehicle following sham surgery. Mice were administered a 7-consecutive-day pulse dosing regimen of 150 μL/day (intravenous tail-vein) of PPF2, recombinant human albumin, or vehicle control. All treatments were initiated 24 hours after CCI or sham surgery.

FIG. 31 reports data from a hot plate test (as described above) thirty-five (35) days post CCI as treated in the timeline of FIG. 24. PPF2-treated mice were significantly less sensitive to hot plate stimuli compared to mice given CCI plus vehicle control. Mice treated with recombinant human albumin were also significantly less sensitive to mice given CCI plus vehicle control, but not to the degree of mice treated with PPF2. *P<0.05 rhAlb vs. vehicle treated CCI mice, ***P<0.001 PPF2 vs. vehicle treated CCI surgery mice. ANOVA with Tukey Post-hoc analysis.

FIG. 32 reports data from a mechanical von Frey allodynia test in these same mice at different time intervals both pre-(baseline) and post-CCI. The pressure at which the mouse withdrew their hind paws was assessed and is represented in FIG. 32 as weeks post CCI or sham surgery. The figure illustrates the mice administered PPF2 following CCI surgery had significantly increased tolerance to mechanical nociception at all assessed timepoints than those treated with vehicle or recombinant human albumin (rhAlb) after CCI. This shows that a PPF (PPF2) ameliorated pain for a greater amount of time than control vehicle or albumin, albumin being the major protein component of PPF. Thus, these effects appear not to be mediated via albumin, but to other proteins present in PPF. *P<0.05; **P<0.01; ***P<0.001; ****P<0.0001 vs. vehicle control; ANOVA with Tukey Post-hoc analysis.

FIG. 33 reports the relative levels of myelin basic protein (MBP, detected by Abcam, ab40390 anti-rabbit antibody) in the distal sciatic nerve five weeks after the last dose of PPF (PPF1) in another similar experiment conducted in 22-month-old mice as described above. *P<0.05; ***P<0.001 vs. vehicle control; ANOVA with Tukey Post-hoc analysis.

FIG. 34 reports the relative levels in these mice of S-100 Schwann cell marker. In both cases, PPF in mice with CCI increased relative levels of these markers compared to vehicle control mice with CCI. Together this shows that PPF promotes sciatic nerve repair mechanisms via increasing myelin protein and S-100 protein expression. It also shows that PPF induces myelination repair mechanisms. **P<0.01; ***P<0.001 vs. vehicle control; ANOVA with Tukey Post-hoc analysis.

FIG. 35 is a fluorescence microscopic qualitative representation of the data reported in FIGS. 33 and 34.

FIG. 36 and FIG. 37 show detection of BDNF and CD68, respectively, in the dorsal horn of the spinal cord in mice treated 24 hours post-CCI injury. Brain-derived neurotrophic factor (BDNF, detected by Abcam, ab108319 anti-rabbit antibody) is secreted by activated microglia and it has been shown to enhance spinal nociception (detection of painful stimuli) through synaptic facilitation and engagement of central sensitization-like mechanisms. Peripheral injury-induced neuropathic pain is often accompanied with increased spinal expression of BDNF (Garraway S M, et al. Neural Plast. Article ID 9857201 (2016)). CD68 levels (detected by Biorad MCA1957 GA anti-rat antibody) were also determined. CD68 is a marker for activated microglia. FIGS. 36 and 37 show that PPF treatment 24 hours after CCI injury results in significant reduction of both BDNF and CD68 markers in the dorsal horns of the spinal cord, indicating the prevention of microglial activation and blocking of deleterious downstream events linked to development of neuropathic pain. **P<0.01; ***P<0.001 vs. vehicle control; ANOVA with Tukey Post-hoc analysis.

FIGS. 38 and 39 are fluorescent microscopic images of the data presented in FIGS. 39 and 37, respectively. The rectangle highlights the dorsal horns of the spinal cord which was analyzed at the L4-L6 lumbar spinal segments. The images on the right sides of the figures are higher focal powered images of the rectangular regions on the left sides of each figure.

b) Pain—Treatment Fourteen Days after Injury

FIG. 40 shows the protocol used on 22-month-old C57BL/6J mice. Baseline von Frey paw withdrawal thresholds for measuring mechanical allodynia were taken 3-4 days before CCI or sham procedures. Cohorts of mice were treated as follows: (1) PPF (PPF1) 14 days following CCI; (2) vehicle 14 days following CCI; (3) recombinant human albumin (rhAlb) 14 days following CCI; or (4) vehicle 14 days following sham surgery. Mice were administered a 7-consecutive-day pulse dosing regimen of 150 μL/day (intravenous tail-vein) of PPF1, recombinant human albumin, or vehicle control. All treatments were initiated 14 days after CCI or sham surgery.

FIG. 41 reports the Von Frey paw withdrawal thresholds at baseline, 14, 21, 28, 35, 42, and 49 days post-CCI. At Day 14, a significant deficit is seen in all but the sham group, indicating that there is central sensitization in all CCI groups after 2 weeks of injury. This is not reversed until 7 days after cessation of treatment with PPF (Day 28), indicating that simple analgesia does not take place with PPF in this model. Instead, a mechanistic effect takes place with PPF treatment which is not observed with vehicle or recombinant human albumin (rh Albumin). This shows that pain that is fully established before PPF treatment (which necessarily involves a central component) is significantly alleviated by PPF compared to vehicle control. **P<0.01; ***P<0.001; ****P<0.0001 vs. vehicle control; ANOVA with Tukey Post-hoc analysis.

FIGS. 42 and 43 report the hot plate latency values at 35 Days post-CCI (FIG. 19) and 49 Days post-CCI (FIG. 20). Both sets of results show that the PPF-treated mice had long-lasting reductions of hot plate pain sensitivity. This also supports the observation that PPF works through a mechanistic effect as opposed to simply providing an analgesic effect. **P<0.01; ANOVA with Tukey Post-hoc analysis.

### Example 3—Improving Nerve Myelination

This Example shows that human plasma fractions such as PPF1 with enhanced safety and tolerability can reverse age-related decline and neuroinflammation in the CNS. This Example also demonstrates that a plasma fraction-based therapeutic approach restores myelination potential in aging and aging-related disease models thereby addressing one or more aging-related conditions such as neuroinflammation and/neurodegeneration.

In vivo mouse models were tested for impairments in myelination and potential utility for testing PPF1 for therapeutic efficacy. The coverage of myelin basic protein (MBP) was compared in both the hippocampus and cortex across various models, including aging, hyperhomocysteinemia (Hhcy), and cisplatin-induced cognitive impairment. Also quantified were hippocampal PDGFRa-expressing oligodendrocyte precursor cells (OPCs) to capture remyelination capacity. The effects of PPF1 on myelination were then tested in aged mice, and an increase in myelin content was found in the hippocampus, which correlated with behavioral performance, as well as cortex. Thus, this Example shows that plasma fractions provide a multimodal therapeutic that have the potential to restore myelin levels in aging and aging-related disease models thereby improving aging-associated behavioral decline.

Histology

Brains are collected following saline perfusion, and fixed hemibrains are sectioned at 30 μm thickness. Free floating sections are blocked with appropriate serum before incubation with primary antibodies at the following concentrations: MBP 1:1000, Abcam; OLIG2, 1:1000, Invitrogen; PDGFRa, 1:500 R&D Systems; CD68, 1:1000, AbD Serotec; Iba-1, 1:2500, Wako; DCX 1:2000 Millipore; BrdU 1:500 (antigen retrieval) Abcam. Y-Maze—The assay chamber consists of a three-armed maze with a start arm cued familiar and novel arms. In a 5 min training session, mice are allowed to explore the start and familiar arms of the maze. After a 4-hour delay, mice are tested with access to all three arms for 5 min, and duration and entries in each arm are recorded.

Image Analysis

Using ImagePro software (Media Cybernetics), the hippocampus, subregion CA1, and cortex are analyzed for MBP percent area coverage and mean optical density using a manual ROI and threshold. OPC density is calculated by counting PDGFRa cells that colocalizes with OLIG2 within the hippocampal ROI. Mice—Hyperhomocysteinemia (Hhcy) was induced in 12-week-old mice via Teklad Custom Diet TD.97345 (Envigo) deficient for folate, vitamins B6 and B12, and supplemented with methionine. For cisplatin studies, 7-month-old mice were dosed with 2.3 mg/kg cisplatin (232120, Calbiochem) via intraperitoneal (IP) injection.

Plasma fraction treatment decreased neuroinflammation and enhanced neurogenesis (FIGS. 44A-44D). FIG. 44A shows a schematic of a fractionation process for plasma fractions. A schematic of study design is shown in FIG. 44B with 22 to 24-month-old wildtype male mice dosed with PPF1 and analyzed 10 days (CD68/Iba-1) or 6 weeks later (BrdU/DCX). PPF1 treatment caused a decrease in microgliosis as shown by quantification of CD68 and Iba-1 immunoreactivity in the hippocampus (FIG. 44C). FIG. 44D shows that PPF1 treatment improves cell survival and neurogenesis. All data shown are mean±SEM; *p<0.05, **p<0.01, ***p<0.001. Veh: Vehicle.

Representative hippocampal images at 11 mo and 24 mo show an age-related decrease of myelin in the hippocampus (FIGS. 45A-45B). The box highlights CA1 ROI shown in the image to the right. Myelin coverage in the hippocampus and cortex did not change from 11 mo to 24 mo (FIG. 45C). The mean optical density of MBP signal is significantly increased in the hippocampus and the CA1 in the 11 mo mice compared to 24 mo mice (FIG. 45D). FIG. 45E provides representative images of PDGFRa⁺ cells in the hippocampus of 11 mo and 24 mo mice. FIG. 45F shows quantification of PDGFRa⁺ cell density in the hippocampus and shows that PDGFRa⁺ cell density in the hippocampus did not change with age. Data shown are mean±SEM; Mann-Whitney Test. **p<0.003. Scale bar=500 μm, 100 μm, 20 μm.

Hhcy and cisplatin models do not show deficits in myelin content. Particularly, FIG. 46A shows a protocol for inducing HHcy in 12-week-old mice via folate-deficient feed for 10 weeks. FIG. 46B shows that no difference was found in percent area coverage of myelin or MBP optical density in the hippocampus or OPC density as measured by PDGFRa in the hippocampus. FIG. 46C shows a schematic of a protocol for inducing cognitive impairment in 7-month-old mice by IP dosing with 2.3 mg/kg cisplatin. FIG. 46D shows that no difference was found in percent area coverage of myelin, optical density in the hippocampus, or OPC density as measured by PDGFRa in the hippocampus. All data shown are mean±SEM.

Aged mice treated with PPF1 show an increased myelin content in the hippocampus and cortex (FIGS. 47A-47G). FIG. 47A shows a schematic of experimental protocol, where 22 mo mice were treated with PPF1 for 7 days, and tissue was collected 10 days later. Hippocampus ROI (inset) and representative dentate gyrus images show an increase in MBP expression in PPF1-treated mice (FIG. 47B). Percent area of myelin coverage and optical density of MBP increased with PPF1 treatment in the hippocampus and CA1 (FIG. 47C). FIG. 47D shows representative images of MBP expression in the cortex and ROI (dotted blue line). FIG. 47E shows that an increased MBP expression is observed in the cortex with PPF1 treatment. FIG. 47F show that no difference in PDGFRa⁺ OPC density was observed in the hippocampus. FIG. 47G shows that a significant correlation was observed between MBP expression with Y-maze performance (percent time in the novel arm) with PPF1 treatment. Spearman Correlation test. R=0.7182, *p=0.0162; Mann-Whitney Test. ****p<0.0001, ***p<0.0002, *p=0.03. Scare bar=200 μm.

These data show that enhancing myelin content provides a viable method for combating age-related cognitive decline. Particularly, PPF1 administration improves various aspects of neuronal health in aged wildtype mice, including increased myelin content.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

What is claimed:

1. A method of restoring myelin levels and/or improving nerve conductance, the method comprising administering an effective amount of a Plasma Fraction to a subject diagnosed with a condition associated with myelin degeneration.

2. The method of claim 1 wherein the Plasma Fraction is a Plasma Protein Fraction (PPF).

3. The method of claim 2 wherein the PPF is a commercially available PPF.

4. The method of claim 2, wherein the PPF has a total protein content that consists of at least 83 percent but less than 95 percent albumin and no more than 17 percent globulins.

5. The method of claim 4 wherein the PPF comprises no more than 1% gamma globulin.

6. The method of claim 1, comprising restoring myelin levels in the subject.

7. The method of claim 1, comprising improving nerve conductance in the subject.

8. The method of claim 1, wherein the condition associated with myelin degeneration is an aging-associated neurodegenerative and/or neuroinflammatory disease.

9. The method of claim 1, wherein the condition associated with myelin degeneration is a myelopathy associated with postoperative recovery.

10. The method claim 1, wherein the Plasma Fraction is derived from plasma obtained from a pool of young individuals.

11. The method claim 1, wherein the Plasma Fraction is produced from a mammalian blood product.

12. The method of claim 11, wherein the mammalian blood product is a human blood product.

13. The method claim 1, wherein the subject is a mammal.

14. The method of claim 13, wherein the mammal is a human.
